(19) United States
(12) Patent Application Publication
(10) Pub. No.: US 2014/0037721 A1
(43) Pub. Date: Feb. 6, 2014

(54) Pharmaceutical Composition

(71) Applicant: Alpharma pharmaceuticals LLC, Bridgewater, NJ (US)

(72) Inventors: Alfred Liang, Bridgewater, NJ (US); Frank Matthews, Bridgewater, NJ (US); Garth Boehm, Bridgewater, NJ (US); Lijuan Tang, Bridgewater, NJ (US); Frank Johnson, Bridgewater, NJ (US); Joseph Stauffer, Bridgewater, NJ (US)

(73) Assignee: Alpharma pharmaceuticals LLC, Bridgewater, NJ (US)

(21) Appl. No.: 14/050,861

(22) Filed: Oct. 10, 2013

Related U.S. Application Data
(63) Continuation of application No. 13/735,523, filed on Jan. 7, 2013, now abandoned.
Application No. 13/735,523 is a continuation of application No. 13/357,723, filed on Jan. 25, 2012, now abandoned.
Application No. 13/357,723 is a continuation of application No. 12/336,418, filed on Dec. 16, 2008, now abandoned.
(60) Provisional application No. 61/007,935, filed on Dec. 17, 2007.

(51) Int. Cl. A61K 31/485; A61K 9/48

(52) U.S. Cl. CPC A61K 31/485; A61K 9/4808
USPC 424/451; 514/282

(57) ABSTRACT

Provided herein are pharmaceutical compositions comprising an antagonist, an agonist, a seal coat, and a sequestering polymer, wherein the antagonist, agonist, seal coat and at least one sequestering polymer are all components of a single unit, and wherein the seal coat forms a layer physically separating the antagonist from the agonist from one another. Methods for manufacturing such a pharmaceutical composition are also provided. Methods for treating pain using such compositions are also demonstrated.

### RELATED APPLICATIONS

This application claims priority to U.S. Ser. No. 60/007,935 filed Dec. 17, 2007.

### FIELD OF THE INVENTION

This invention pertains to compositions and methods useful for treating pain in human patients. One such composition contains both an opioid antagonist and an opioid agonist formulated such that the agonist is released over time with minimal release of the antagonist.

### BACKGROUND OF THE INVENTION

Improved methods for treating pain are desired by those of skill in the art. A disease in which pain is a major symptom is osteoarthritis (OA). OA is the most common form of arthritis in the United States (Hochberg et al., 1995a), affecting more than 21 million people. It is a disease of primarily middle-aged and older adults and is a leading cause of disability (American College of Rheumatology, 2000a). OA results from degeneration of the joint cartilage, and usually involves the neck, low back, knees, hips, and fingers. The prevalence of OA of the hip and knee increases progressively with age (Peloso et al. 2000). Unlike rheumatoid arthritis and other inflammatory arthritides, inflammation, if present, is usually mild and localized to the joint. The cause of OA is unknown, but biomechanical stresses affecting the articular cartilage and subchondral bone, biochemical changes in the articular cartilage and synovial membrane, and genetic factors are significant in its pathogenesis (Hochberg et al., 1995b; American College of Rheumatology, 2000b).

OA is characterized by pain that typically worsens with activity and weight bearing and improves with rest, as well as morning stiffness, and pain and stiffness that ease after a few minutes of movement. Clinical examination often reveals tenderness to palpation, bony enlargement, crepitus, and/or limited joint motion (American College of Rheumatology, 2000b). As the disease advances, OA patients experience increasing pain and loss of function, with pain intruding at periods of rest (Peloso et al. 2000). Since no cure for OA is available, the primary goal of OA treatment is to reduce pain while maintaining or improving joint mobility and limiting functional impairment.

Nonpharmacologic and pharmacologic treatments for OA are used in conjunction to reduce pain and to improve functional status. Nonpharmacologic therapies include patient education, weight loss (if overweight), occupational therapy, physical therapy, and aerobic exercise programs to restore joint movement and increase strength and aerobic capacity (American College of Rheumatology, 2000a). The initial pharmacologic therapies for OA include nonopioid analgesics (e.g., acetaminophen) and topical analgesics, followed by treatment with nonsteroidal anti-inflammatory drugs (NSAIDs) and judicious use of intra-articular steroid injections (Hochberg et al., 1995a). Although these medications may provide temporary pain relief the beneficial effect may be offset by other factors. Use of nonopioid analgesics to treat moderate to severe OA pain is limited by a ceiling effect for analgesia (Roth et al., 2000). Additionally, NSAIDs can be toxic to the gastrointestinal tract, and NSAIDs and acetaminophen can produce renal toxicity, especially in the elderly (Peloso et al. 2000). Thus, a need exists for additional analgesic treatment options for pain associated with OA.

Recent efforts have been made to liberalize the use of opioids for the treatment of chronic nonmalignant pain (Sullivan et al., 2005). Sullivan proposes subject-centered principles to guide efforts to relieve chronic nonmalignant pain, including the acceptance of all subject pain reports as valid but negotiation of treatment goals early in care, avoidance of subject harm, and incorporation of chronic opioids as one part of the treatment plan if they improve the subject's overall health-related quality of life. Prescribing opiates in the treatment of chronic nonmalignant pain may pose a challenge to the primary care physician (Olsen et al., 2004).

Although an outright ban on opioid use in chronic nonmalignant pain is no longer ethically acceptable, ensuring that opioids provide overall benefit to subjects requires significant physician time and skill. Subjects with chronic nonmalignant pain should be assessed and treated for concurrent psychiatric disorders; those with disorders are entitled to equivalent efforts at pain relief. The essential question is not whether chronic nonmalignant pain is real or proportional to objective disease severity, but how it should be managed so that the subject's overall quality of life is optimized.

As early as the mid 1990s, naltrexone has been shown to effectively block morphine effects in humans (Kaiko et al., 1995). Morphine effects in normal volunteers were blocked by three 100-mg doses of naltrexone. The first dose of naltrexone was given 24 hours before dosing with controlled release morphine sulfate (MS Contin®), followed by a second dose at the time of MS Contin dosing and a third dose 24 hours after MS Contin administration. Single 200 mg doses of MS Contin given with the naltrexone blockade were generally well tolerated, and adverse effects were similar to those reported for naltrexone alone and for lower doses of morphine without naltrexone. Naltrexone proved safe and effective in blocking the effects of controlled release morphine, permitting bioequivalence studies of a high dose of morphine in normal volunteers.

Although well absorbed orally, naltrexone is subject to significant first-pass metabolism, with oral bioavailability estimates ranging from 5% to 40% (Naltrexone HCl Tablets, USP Package Insert). The activity of naltrexone is believed to be due to both the parent compound and the 6-β-naltrexol metabolite. Both parent drug and metabolites are excreted primarily by the kidney (53% to 79% of the dose); however, urinary excretion of unchanged naltrexone accounts for less than 2% of an oral dose and fecal excretion is a minor elimination pathway. The mean elimination terminal half-life (t_{1:2}) values for naltrexone and 6-β-naltrexol are 4 hours and 13 hours, respectively. Naltrexone and 6-β-naltrexol are dose-proportional in terms of area under the concentration-time curve (AUC) and maximum plasma concentration (Cₘₐₓ) over the range of 50 to 200 mg and do not accumulate after 100 mg daily doses.

Various formulations of opioids are in development that have a reduced risk of diversion and non-medical use and can be used to treat patients with chronic, nonmalignant conditions. Kadian® (morphine sulfate extended-release capsule) was developed for use in subjects with chronic pain who require repeated dosing with a potent opioid analgesic, and has been tested in subjects with pain due to malignant and nonmalignant conditions. Kadian contains polymer-coated extended-release pellets of morphine sulfate, to deliver up to 24 hours of continuous pain relief. This formulation lacks an immediate-release component, only providing a slow release of the analgesic. This slow-release technology serves to minimize plasma peaks and troughs, thereby providing a relatively flat pharmacokinetic (PK) curve upon multiple dosing. This delivery mechanism is ideally suited for chronic pain patients. Kadian capsules are an extended-release oral formulation of morphine sulfate indicated for the management of moderate to severe pain when a continuous, around-the-clock opioid analgesic is needed for an extended period of time.

However, persons abusing opioids are likely to tamper with controlled-release formulations in hopes of obtaining the entire dose to induce an immediate euphoria. To further deter non-medical opioid use, formulations containing opioid antagonists are being developed. As described herein, Kadian NT (morphine sulfate plus naltrexone hydrochloride extended-release capsules), is a product that is intended to be used as an opiate analgesic for moderate to severe pain. Its abuse-deterrence feature incorporates an immediate release of naltrexone upon illicit manipulation; this is intended to neutralize the euphoric potential of morphine and increase safety after ingestion of the tampered product. If Kadian NT is used as directed, a patient should receive a dose of morphine equivalent to the same mg dose of Kadian. However, if the drug product is tampered with and ingested by a patient who is opioid dependent, the patient may be exposed to a dose of naltrexone sufficient to produce withdrawal symptoms.

Abuse-resistant, sustained-release dosage forms of products intended to treat pain have been described in the art (see, for example, U.S. Application Nos. 2003/0124185 and 2003/0044458). However, it is believed that substantial amounts of the opioid antagonist or other antagonist found in these sequestered forms are released over time (usually less than 24 hours) due to the osmotic pressure that builds up in the core of the sequestered form, as water permeates through the sequestered form into the core. The high osmotic pressure inside the core of the sequestered form causes the opioid antagonist or antagonist to be pushed out of the sequestered form, thereby causing the opioid antagonist or antagonist to be released from the sequestered form. As shown below, certain embodiments described herein provide improved forms of sequestered opioid antagonists and controlled-release opioid agonists.

In view of the foregoing drawbacks of the sequestered forms of the prior art, there exists a need in the art for methods of treating pain. A sequestered form of an opioid antagonist or other antagonist that is not substantially released from the sequestered form due to osmotic pressure. The invention provides such a sequestering form of an opioid antagonist or antagonist. This and other objects and advantages of the invention, as well as additional inventive features, will be apparent from the description of the invention provided herein.

### BRIEF DESCRIPTION OF THE DRAWINGS

FIG. 1. Mean Change From Baseline BPI Average Pain Score in the ITT Population.

FIG. 2. BPI Diary Average Pain Score.

FIG. 3. WOMAC Pain Score.

FIG. 4. WOMAC Composite Score.

### BRIEF SUMMARY OF THE INVENTION

This invention pertains to compositions and methods useful for treating pain in human patients. One such composition contains both an opioid antagonist and an opioid agonist formulated such that the agonist is released over time with minimal release of the antagonist.

### DETAILED DESCRIPTION OF THE INVENTION

Provided herein are compositions and methods for administering a multiple active agents to a mammal in a form and manner that minimizes the effects of either active agent upon the other in vivo. In certain embodiments, at least two active agents are formulated as part of a pharmaceutical composition. A first active agent may provide a therapeutic effect in vivo. The second active agent may be an antagonist of the first active agent, and may be useful in preventing misuse of the composition. For instance, where the first active agent is a narcotic, the second active agent may be an antagonist of the narcotic. The composition remains intact during normal usage by patients and the antagonist is not released. However, upon tampering with the composition, the antagonist may be released thereby preventing the narcotic from having its intended effect. In certain embodiments, the active agents are both contained within a single unit, such as a bead, in the form of layers. The active agents may be formulated with a substantially impermeable barrier as, for example, a controlled-release composition, such that release of the antagonist from the composition is minimized. In certain embodiments, the antagonist is released in in vitro assays but is substantially not released in vivo. In vitro and in vivo release of the active agent from the composition may be measured by any of several well-known techniques. For instance, in vitro release may be determined by measuring the plasma levels of the active agent or metabolites thereof (i.e., AUC, Cmax).

In certain embodiments, one of the active agents is an opioid receptor agonist. Several opioid agonists are commercially available or in clinical trials and may be administered as described herein such that the alcohol effects are minimized. Opioid agonists include, for example, alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydroetorphine, dihydromorphine, dimencoadol, dimepheptanol, dimethylthiambutene, dioxaphetyl butyrate, dipipanone, eptazocine, ethoheptazine, ethylmethylthiambutene, ethylmorphine, etonitazene, etorphine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenazocine, phenomorphan, phenoperidine, piminodine, piritramide, propheptazine, promedol, properidine, propiram, propoxyphene, sufentanil, tramadol, tilidine, derivatives or complexes thereof, pharmaceutically acceptable salts thereof, and combinations thereof. Preferably, the opioid agonist is selected from the group consisting of hydrocodone, hydromorphone, oxycodone, dihydrocodeine, codeine, dihydromorphine, morphine, buprenorphine, derivatives or complexes thereof, pharmaceutically acceptable salts thereof, and combinations thereof. Most preferably, the opioid agonist is morphine, hydromorphone, oxycodone or hydrocodone. Equianalgesic doses of these opioids, in comparison to a 15 mg dose of hydrocodone, are as follows: oxycodone (13.5 mg), codeine (90.0 mg), hydrocodone (15.0 mg), hydromorphone (3.375 mg), levorphanol (1.8 mg), meperidine (135.0 mg), methadone (9.0 mg), and morphine (27.0 mg).

A common dosage form of hydrocodone is in combination with acetaminophen and is commercially available, for example, as Lortab® in the United States from UCB Pharma, Inc. (Brussels, Belgium), as 2.5/500 mg, 5/500 mg, 7.5/500 mg and 10/500 mg hydrocodone/acetaminophen tablets. Tablets are also available in the ratio of 7.5 mg hydrocodone bitartrate and 650 mg acetaminophen and a 7.5 mg hydrocodone bitartrate and 750 mg acetaminophen. Hydrocodone, in combination with aspirin, is given in an oral dosage form to adults generally in 1-2 tablets every 4-6 hours as needed to alleviate pain. The tablet form is 5 mg hydrocodone bitartrate and 224 mg aspirin with 32 mg caffeine; or 5 mg hydrocodone bitartrate and 500 mg aspirin. Another formulation comprises hydrocodone bitartrate and ibuprofen. Vicoprofen®, commercially available in the U.S. from Knoll Laboratories (Mount Olive, N.J.), is a tablet containing 7.5 mg hydrocodone bitartrate and 200 mg ibuprofen. The invention is contemplated to encompass all such formulations, with the inclusion of the opioid antagonist and/or antagonist in sequestered form as past of a subunit comprising an opioid agonist.

Oxycodone, chemically known as 4,5-epoxy-14-hydroxy-3-methoxy-17-methylmorphinan-6-one, is an opioid agonist whose principal therapeutic action is analgesia. Other therapeutic effects of oxycodone include anxiolysis, euphoria and feelings of relaxation. The precise mechanism of its analgesic action is not known, but specific CNS opioid receptors for endogenous compounds with opioid-like activity have been identified throughout the brain and spinal cord and play a role in the analgesic effects of this drug. Oxycodone is commercially available in the United States, e.g., as Oxycotin® from Purdue Pharma L.P. (Stamford, Conn.), as controlled-release tablets for oral administration containing 10 mg, 20 mg, 40 mg or 80 mg oxycodone hydrochloride, and as OxyIR™, also from Purdue Pharma L.P., as immediate-release capsules containing 5 mg oxycodone hydrochloride. The invention is contemplated to encompass all such formulations, with the inclusion of an opioid antagonist and/or antagonist in sequestered form as part of a subunit comprising an opioid agonist.

Oral hydromorphone is commercially available in the United States, e.g., as Dilaudid® from Abbott Laboratories (Chicago, Ill.). Oral morphine is commercially available in the United States, e.g., as Kadian® from Faulding Laboratories (Piscataway, N.J.).

In embodiments in which the opioid agonist comprises hydrocodone, the sustained-release oral dosage forms can include analgesic doses from about 8 mg to about 50 mg of hydrocodone per dosage unit. In sustained-release oral dosage forms where hydromorphone is the therapeutically active opioid, it is included in an amount from about 2 mg to about 64 mg hydromorphone hydrochloride. In another embodiment, the opioid agonist comprises morphine, and the sustained-release oral dosage forms of the invention include from about 2.5 mg to about 800 mg morphine, by weight. In yet another embodiment, the opioid agonist comprises oxycodone and the sustained-release oral dosage forms include from about 2.5 mg to about 800 mg oxycodone. In certain preferred embodiments, the sustained-release oral dosage forms include from about 20 mg to about 30 mg oxycodone. Controlled release oxycodone formulations are known in the art. The following documents describe various controlled-release oxycodone formulations suitable for use in the invention described herein, and processes for their manufacture: U.S. Pat. Nos. 5,266,331; 5,549,912; 5,508,042; and 5656,295, which are incorporated herein by reference. The opioid agonist can comprise tramadol and the sustained-release oral dosage forms can include from about 25 mg to 800 mg tramadol per dosage unit.

In certain embodiments, another active agent contained within the composition may be an opioid receptor antagonist. In certain embodiments, the agonist and antagonist are administered together, either separately or as part of a single pharmaceutical unit. In the instance when the therapeutic agent is an opioid agonist, the antagonist preferably is an opioid antagonist, such as naltrexone, naloxone, nalmefene, cyclazocine, levallorphan, derivatives or complexes thereof, pharmaceutically acceptable salts thereof, and combinations thereof. More preferably, the opioid antagonist is naloxone or naltrexone. By “opioid antagonist” is meant to include one or more opioid antagonists, either alone or in combination, and is further meant to include partial antagonists, pharmaceutically acceptable salts thereof, stereoisomers thereof, ethers thereof, esters thereof, and combinations thereof. The pharmaceutically acceptable salts include metal salts, such as sodium salt, potassium salt, cesium salt, and the like; alkaline earth metals, such as calcium salt, magnesium salt, and the like; organic amine salts, such as triethylamine salt, pyridine salt, picoline salt, ethanolamine salt, triethanolamine salt, dicyclohexylamine salt, N,N-dibenzylethylenediamine salt, and the like; inorganic acid salts, such as hydrochloride, hydrobromide, sulfate, phosphate, and the like; organic acid salts, such as formate, acetate, trifluoroacetate, maleate, tartrate, and the like; sulfonates, such as methanesulfonate, benzenesulfonate, p-toluenesulfonate, and the like; amino acid salts, such as arginate, asparaginate, glutamate, and the like. In certain embodiments, the amount of the opioid antagonist can be about 10 ng to about 275 mg. In a preferred embodiment, when the antagonist is naltrexone, it is preferable that the intact dosage form releases less than 0.125 mg or less within 24 hours, with 0.25 my or greater of naltrexone released after 1 hour when the dosage form is crushed or chewed.

In a preferred embodiment, the opioid antagonist comprises naloxone. Naloxone is an opioid antagonist, which is almost void of agonist effects. Subcutaneous doses of up to 12 mg of naloxone produce no discernable subjective effects, and 24 mg naloxone causes only slight drowsiness. Small doses (0.4-0.8 mg) of naloxone given intramuscularly or intravenously in man prevent or promptly reverse the effects of morphine-like opioid agonist. One mg of naloxone intravenously has been reported to block completely the effect of 25 mg of heroin. The effects of naloxone are seen almost immediately after intravenous administration. The drug is absorbed after oral administration, but has been reported to be metabolized into an inactive form rapidly in its first passage through the Liver, such that it has been reported to have significantly lower potency than when parenterally administered. Oral dosages of more than 1 g have been reported to be almost completely metabolized in less than 24 hours. It has been reported that 25% of naloxone administered sublingually is absorbed (Weinberg et al., Clin. Pharmacol. Ther. 44:335-340 (1988)).

In another preferred embodiment, the opioid antagonist comprises naltrexone. In the treatment of patients previously addicted to opioids, naltrexone has been used in large oral doses (over 100 mg) to prevent euphorigenic effects of opioid agonists. Naltrexone has been reported to exert strong preferential blocking action against mu over delta sites. Naltrexone is known as a synthetic congener of oxymorphone with no opioid agonist properties, and differs in structure from oxymorphone by the replacement of the methyl group located on the nitrogen atom of oxymorphone with a cyclopropylmethyl group. The hydrochloride salt of naltrexone is soluble in water up to about 100 mg/cc. The pharmacological and pharmacokinetic properties of naltrexone have been evaluated in multiple animal and clinical studies. See, e.g., Gonzalez et al. Drugs 35:192-213 (1988). Following oral administration, naltrexone is rapidly absorbed (within 1 hour) and has an oral bioavailability ranging from 5-40%. Naltrexone's protein binding is approximately 21% and the volume of distribution following single-dose administration is 16.1 L/kg.

Naltrexone is commercially available in tablet form (Revia®, DuPont (Wilmington, Del.)) for the treatment of alcohol dependence and for the blockade of exogenously administered opioids. See, e.g., Revia (naltrexone hydrochloride tablets), Physician's Desk Reference, 51^{st} ed., Montvale, N.J.; and Medical Economics 51:957-959 (1997). A dosage of 50 mg Revia® blocks the pharmacological effects of 25 mg IV administered heroin for up to 24 hours. It is known that, when coadministered with morphine, heroin or other opioids on a chronic basis, naltrexone blocks the development of physical dependence to opioids. It is believed that the method by which naltrexone blocks the effects of heroin is by competitively binding at the opioid receptors. Naltrexone has been used to treat narcotic addiction by complete blockade of the effects of opioids. It has been found that the most successful use of naltrexone for a narcotic addiction is with narcotic addicts having good prognosis, as part of a comprehensive occupational or rehabilitative program involving behavioral control or other compliance-enhancing methods. For treatment of narcotic dependence with naltrexone, it is desirable that the patient be opioid-free for at least 7-10 days. The initial dosage of naltrexone for such purposes has typically been about 25 mg, and if no withdrawal signs occur, the dosage may be increased to 50 mg per day. A daily dosage of 50 mg is considered to produce adequate clinical blockade of the actions of parenterally administered opioids. Naltrexone also has been used for the treatment of alcoholism as an adjunct with social and psychotherapeutic methods.

Other preferred opioid antagonists include, for example, cyclazocine and naltrexone, both of which have cyclopropylmethyl substitutions on the nitrogen, retain much of their efficacy by the oral route, and last longer, with durations approaching 24 hours after oral administration.

The antagonist may also be a bittering agent. The term “bittering agent” as used herein refers to any agent that provides an unpleasant taste to the host upon inhalation and/or swallowing of a tampered dosage form comprising the sequestering subunit. With the inclusion of a bittering agent, the intake of the tampered dosage form produces a bitter taste upon inhalation or oral administration, which, in certain embodiments, spoils or hinders the pleasure of obtaining a high from the tampered dosage form, and preferably prevents the abuse of the dosage form.

Various bittering agents can be employed including, for example, and without limitation, natural, artificial and synthetic flavor oils and flavoring aromatics and/or oils, oleoresins and extracts derived from plants, leaves, flowers, fruits, and so forth, and combinations thereof. Nonlimiting representative flavor oils include spearmint oil, peppermint oil, eucalyptus oil, oil of nutmeg, allspice, mace, oil of bitter almonds, menthol and the like. Also useful bittering agents are artificial, natural and synthetic fruit flavors such as citrus oils, including lemon, orange, lime, and grapefruit, fruit essences, and so forth. Additional bittering agents include sucrose derivatives (e.g., sucrose octaacetate), chlorosucrose derivatives, quinine sulphate, and the like. A preferred bittering agent for use in the invention is Denatonium Benzoate NF-Anhydrous, sold under the name Bitrex™ (Macfarlan Smith Limited, Edinburgh, UK). A bittering agent can be added to the formulation in an amount of less than about 50% by weight, preferably less than about 10% by weight, more preferably less than about 5% by weight of the dosage form, and most preferably in an amount ranging from about 0.1 to 1.0 percent by weight of the dosage form, depending on the particular bittering agent(s) used.

Alternatively, the antagonist may be a dye. The term “dye” as used herein refers to any agent that causes discoloration of the tissue in contact. In this regard, if the sequestering subunit is tampered with and the contents are snorted, the dye will discolor the nasal tissues and surrounding tissues thereof. Preferred dyes are those that can bind strongly with subcutaneous tissue proteins and are well-known in the art. Dyes useful in applications ranging from, for example, food coloring to tattooing, are exemplary dyes suitable for the invention. Food coloring dyes include, but are not limited to FD&C Green #3 and FD&C Blue #1, as well as any other FD&C or D&C color. Such food dyes are commercially available through companies, such as Voigt Global Distribution (Kansas City, Mo.).

The antagonist may alternatively be an irritant. The term “irritant” as used herein includes a compound used to impart an irritating, e.g., burning or uncomfortable, sensation to an abuser administering a tampered dosage form of the invention. Use of an irritant will discourage an abuser from tampering with the dosage form and thereafter inhaling, injecting, or swallowing the tampered dosage form. Preferably, the irritant is released when the dosage form is tampered with and provides a burning or irritating effect to the abuser upon inhalation, injection, and/or swallowing the tampered dosage form. Various irritants can be employed including, for example, and without limitation, capsaicin, a capsaicin analog with similar type properties as capsaicin, and the like. Some capsaicin analogues or derivatives include, for example, and without limitation, resiniferatoxin, tinyatoxin, heptanoylisobutylamide, heptanoyl guaiacylamide, other isobutylamides or guaiacylamides, dihydrocapsaicin, homovanillyl octylester, nonanoyl vanillylamide, or other compounds of the class known as vanilloids. Resiniferatoxin is described, for example, in U.S. Pat. No. 5,290,816. U.S. Pat. No. 4,812,446 describes capsaicin analogs and methods for their preparation. Furthermore, U.S. Pat. No. 4,424,205 cites Newman, “Natural and Synthetic Pepper-Flavored Substances,” published in 1954 as listing pungency of capsaicin-like analogs. Ton et al. British Journal of Pharmacology 10:175-182 (1955), discusses pharmacological actions of capsaicin and its analogs. With the inclusion of an irritant (e.g., capsaicin) in the dosage form, the irritant imparts a burning or discomforting quality to the abuser to discourage the inhalation, injection, or oral administration of the tampered dosage form and preferably to prevent the abuse of due dosage form. Suitable capsaicin compositions include capsaicin (trans 8-methyl-N-vanillyl-6-noneamide) or analogues thereof in a concentration between about 0.00125% and 50% by weight, preferably between about 1% and about 7.5% by weight, and most preferably, between about 1% and about 5% by weight.

The antagonist may also be a gelling agent. The term “gelling agent” as used herein refers to any agent that provides a gel-like quality to the tampered dosage form, which slows the absorption of the therapeutic agent, which is formulated with the sequestering subunit, such that a host is less likely to obtain a rapid “high.” In certain preferred embodiments, when the dosage form is tampered with and exposed to a small amount (e.g., less than about 10 ml) of an aqueous liquid (e.g., water), the dosage form will be unsuitable for injection and/or inhalation. Upon the addition of the aqueous liquid, the tampered dosage form preferably becomes thick and viscous, rendering it unsuitable for injection. The term “unsuitable for injection” is defined for purposes of the invention to mean that one would have substantial difficulty injecting the dosage form (e.g., due to pain upon administration or difficulty pushing the dosage form through a syringe) due to the viscosity imparted on the dosage form, thereby reducing the potential for abuse of the therapeutic agent in the dosage form. In certain embodiments, the gelling agent is present in such an amount in the dosage form that attempts at evaporation (by the application of heat) to an aqueous mixture of the dosage form in an effort to produce a higher concentration of the therapeutic agent, produces a highly viscous substance unsuitable for injection. When nasally inhaling the tampered dosage form, the gelling agent can become gel-like upon administration to the nasal passages, due to the moisture of the mucous membranes. This also makes such formulations aversive to nasal administration, as the gel will stick to the nasal passage and minimize absorption of the abusable substance. Various gelling agents may can be employed including, for example, and without limitation, sugars or sugar-derived alcohols, such as mannitol, sorbitol, and the like, starch and starch derivatives, cellulose derivatives, such as microcrystalline cellulose, sodium carboxymethyl cellulose, methylcellulose, ethyl cellulose, hydroxyethyl cellulose, hydroxypropyl cellulose, and hydroxypropyl methylcellulose, attapulgites, bentonites, dextrins, alginates, carrageenan, gum tragacant, gum acacia, guar gum, xanthan gum, pectin, gelatin, kaolin, lecithin, magnesium aluminum silicate, the carbomers and carbopols, polyvinylpyrrolidone, polyethylene glycol, polyethylene oxide, polyvinyl alcohol, silicon dioxide, surfactants, mixed surfactant/wetting agent systems, emulsifiers, other polymeric materials, and mixtures thereof, etc. In certain preferred embodiments, the gelling agent is xanthan gum. In other preferred embodiments, the gelling agent of the invention is pectin. The pectin or pectic substances useful for this invention include not only purified or isolated pectates but also crude natural pectin sources, such as apple, citrus or sugar beet residues, which have been subjected, when necessary, to esterification or de-esterification, e.g., by alkali or enzymes. Preferably, the pectins used in this invention are derived from citrus fruits, such as lime, lemon, grapefruit, and orange. With the inclusion of a gelling agent in the dosage form, the gelling agent preferably imparts a gel-like quality to the dosage form upon tampering that spoils or hinders the pleasure of obtaining a rapid high from due to the gel-like consistency of the tampered dosage form in contact with die mucous membrane, and in certain embodiments, prevents the abuse of the dosage form by minimizing absorption, e.g., in the nasal passages. A gelling agent can be added to the formulation in a ratio of gelling agent to opioid agonist of from about 1:40 to about 40:1 by weight, preferably from about 1:1 to about 30:1 by weight, and more preferably from about 2:1 to about 10:1 by weight of the opioid agonist. In certain other embodiments, the dosage form forms a viscous gel having a viscosity of at least about 10 cP after the dosage form is tampered with by dissolution in an aqueous liquid (from about 0.5 to about 10 ml and preferably from 1 to about 5 ml). Most preferably, the resulting mixture will have a viscosity of at least about 60 cP.

The antagonist can comprise a single type of antagonist (e.g., a capsaicin), multiple forms of a single type of antagonist (e.g., a capasin and an analogue thereof), or a combination of different types of antagonists (e.g., one or more bittering agents and one or more gelling agents). Desirably, the amount of antagonist in a unit of the invention is not toxic to the host.

In one embodiment, the invention provides a sequestering subunit comprising an opioid antagonist and a blocking agent, wherein the blocking agent substantially prevents release of the opioid antagonist from the sequestering subunit in the gastrointestinal tract for a time period that is greater than 24 hours. This sequestering subunit is incorporated into a single pharmaceutical unit that also includes an opioid agonist. The pharmaceutical unit thus includes a core portion to which the opioid antagonist is applied. A seal coat is then optionally applied upon the antagonist. Upon the seal coat is then applied a composition comprising the pharmaceutically active agent. An additional layer containing the same or a different blocking agent may then be applied such that the opioid agonist is released in the digestive tract over time (i.e., controlled release). Thus, the opioid antagonist and the opioid agonist are both contained within a single pharmaceutical unit, which is typically in the form of a bead.

The term “sequestering subunit” as used herein refers to any means for containing an antagonist and preventing or substantially preventing the release thereof in the gastrointestinal tract when intact, i.e., when not tampered with. The term “blocking agent” as used herein refers to the means by which the sequestering subunit is able to prevent substantially the antagonist from being released. The blocking agent may be a sequestering polymer, for instance, as described in greater detail below.

The terms “substantially prevents,” “prevents,” or any words stemming therefrom, as used herein, means that the antagonist is substantially not released from the sequestering subunit in the gastrointestinal tract. By “substantially not released” is meant that the antagonist may be released in a small amount, but the amount released does not affect or does not significantly affect the analgesic efficacy when the dosage form is orally administered to a host, e.g., a mammal (e.g., a human), as intended. The terms “substantially prevents,” “prevents,” or any words stemming therefrom, as used herein, does not necessarily imply a complete or 100% prevention. Rather, there are varying degrees of prevention of which one of ordinary skill in the art recognizes as having a potential benefit. In this regard, the blocking agent substantially prevents or prevents the release of the antagonist to the extent that at least about 80% of the antagonist is prevented from being released from the sequestering subunit in the gastrointestinal tract for a time period that is greater than 24 hours. Preferably, the blocking agent prevents release of at least about 90% of the antagonist from the sequestering subunit in the gastrointestinal tract for a time period that is greater than 24 hours. More preferably, the blocking agent prevents release of at least about 95% of the antagonist from the sequestering subunit. Most preferably, the blocking agent prevents release of at least about 99% of the antagonist from the sequestering subunit in the gastrointestinal tract for a time period that is greater than 24 hours.

For purposes of this invention, the amount of the antagonist released after oral administration can be measured in-vitro by dissolution testing as described in the United States Pharmacopeia (USP26) in chapter <711> Dissolution. For example, using 900 mL of 0.1N HCl, Apparatus 2 (Paddle), 75 rpm, at 37° C. to measure release at various times from the dosage unit. Other methods of measuring the release of an antagonist from a sequestering subunit over a given period of time are known in the art (see, e.g., USP26).

Without being bound to any particular theory, it is believed that the sequestering subunit of the invention overcomes the limitations of the sequestered forms of an antagonist known in the art in that the sequestering subunit of the invention reduces osmotically-driven release of the antagonist from the sequestering subunit. Furthermore, it is believed that the present inventive sequestering subunit reduces the release of the antagonist for a longer period of time (e.g., greater than 24 hours) in comparison to the sequestered forms of antagonists known in the art. The fact that the sequestered subunit of the invention provides a longer prevention of release of the antagonist is particularly relevant, since precipitated withdrawal could occur after the time for which the therapeutic agent is released and acts. It is well known that the gastrointestinal tract transit time for individuals varies greatly within the population. Hence, the residue of the dosage form may be retained in the tract for longer than 24 hours, and in some cases for longer than 48 hours. It is further well known that opioid analgesics cause decreased bowel motility, further prolonging gastrointestinal tract transit time. Currently, sustained-release forms having an effect over a 24 hour time period have been approved by the Food and Drug Administration. In this regard, the present inventive sequestering subunit provides prevention of release of the antagonist for a time period that is greater than 24 hours when the sequestering subunit has not been tampered.

The sequestering subunit of the invention is designed to prevent substantially the release of the antagonist when intact. By “intact” is meant that a dosage form has not undergone tampering. The term “tampering” is meant to include any manipulation by mechanical, thermal and/or chemical means, which changes the physical properties of the dosage form. The tampering can be, for example, crushing, shearing, grinding, chewing, dissolution in a solvent, heating (for example, greater than about 45° C.), or any combination thereof. When the sequestering subunit of the invention has been tampered with, the antagonist is released from the sequestering subunit. In some cases, the release is immediate.

By “subunit” is meant to include a composition, mixture, particle; etc., that can provide a dosage form (e.g., an oral dosage form) when combined with another subunit. The subunit can be in the form of a bead, pellet, granule, spheroid, or the like, and can be combined with additional same or different subunits, in the form of a capsule, tablet or the like, to provide a dosage form, e.g., an oral dosage form. The subunit may also be part of a larger, single unit, forming part of that unit, such as a layer. For instance, the subunit may be a core coated with an antagonist and a seal coat; this subunit may then be coated with additional compositions including a pharmaceutically active agent such as an opioid agonist.

For purposes of the invention, the antagonist can be any agent that negates the effect of the therapeutic agent or produces an unpleasant or punishing stimulus or effect, which will deter or cause avoidance of tampering with the sequestering subunit or compositions comprising the same. Desirably, the antagonist does not harm a host by its administration or consumption but has properties that deter its administration or consumption, e.g., by chewing and swallowing or by crushing and snorting, for example. The antagonist can have a strong or foul taste or smell, provide a burning or tingling sensation, cause a lachrymation response, nausea, vomiting, or any other unpleasant or repugnant sensation, or color tissue, for example. Preferably, the antagonist is selected from the group consisting of an antagonist of a therapeutic agent, a bittering agent, a dye, a gelling agent, and an irritant. Exemplary antagonists include capsaicin, dye, bittering agents and emetics.

By “antagonist of a therapeutic agent” is meant any drug or molecule, naturally-occurring or synthetic, that binds to the same target molecule (e.g., a receptor) of the therapeutic agent, yet does not produce a therapeutic, intracellular, or in vivo response. In this regard, the antagonist of a therapeutic agent binds to the receptor of the therapeutic agent, thereby preventing the therapeutic agent front acting on the receptor, thereby preventing the achievement of a “high” in the host.

In the instance when the therapeutic agent is an opioid agonist, the antagonist preferably is an opioid antagonist, such as naltrexone, naloxone, nalmefene, cyclazocine, levallorphan, derivatives or complexes thereof, pharmaceutically acceptable salts thereof, and combinations thereof. More preferably, the opioid antagonist is naloxone or naltrexone. By “opioid antagonist” is meant to include one or more opioid antagonists, either alone or in combination, and is further meant to include partial antagonists, pharmaceutically acceptable salts thereof, stereoisomers thereof, ethers thereof, esters thereof, and combinations thereof. The pharmaceutically acceptable salts include metal salts, such as sodium salt, potassium salt, cesium salt, and the like; alkaline earth metals, such as calcium salt, magnesium salt, and the like; organic amine salts, such as triethylamine salt, pyridine salt, picoline salt, ethanolamine salt, triethanolamine salt, dicyclohexylamine salt, N,N-dibenzylethylenediamine salt, and the like; inorganic acid salts, such as hydrochloride, hydrobromide, sulfate, phosphate, and the like; organic acid salts, such as formate, acetate, trifluoroacetate, maleate, tartrate, and the like; sulfonates, such as methanesulfonate, benzenesulfonate, p-toluenesulfonate, and the like; amino acid salts, such as arginate, asparaginate, glutamate, and the like. In certain embodiments, the amount of the opioid antagonist, present in sequestered form, can be about 10 ng to about 275 mg. In a preferred embodiment, when the antagonist is naltrexone, it is preferable that the intact dosage form releases in vivo less than 0.125 mg or less within 24 hours, with 0.25 mg or greater of naltrexone released after 1 hour when the dosage form is crushed or chewed.

The antagonist can comprise a single type of antagonist (e.g., a capsaicin), multiple forms of a single type of antagonist (e.g., a capasin and an analogue thereof), or a combination of different types of antagonists (e.g., one or more bittering agents and one or more gelling agents). Desirably, the amount of antagonist in the sequestering subunit of the invention is not toxic to the host.

The blocking agent prevents or substantially prevents the release of the antagonist in the gastrointestinal tract for a time period that is greater than 24 hours, e.g., between 24 and 25 hours, 30 hours, 35 hours, 40 hours 45 hours, 48 hours, 50 hours, 55 hours, 60 hours, 65 hours, 70 hours, 72 hours, 75 hours, 80 hours, 85 hours, 90 hours, 95 hours, or 100 hours; etc. Preferably, the time period for which the release of the antagonist is prevented or substantially prevented in the gastrointestinal tract is at least about 48 hours. More preferably, the blocking agent prevents or substantially prevents the release for a time period of at least about 72 hours.

The blocking agent of the present inventive sequestering subunit can be a system comprising a first antagonist-impermeable material and a core. By “antagonist-impermeable material” is meant any material that is substantially impermeable to the antagonist, such that the antagonist is substantially not released from the sequestering subunit. The term “substantially impermeable” as used herein does not necessarily imply complete or 100% impermeability. Rather, there are varying degrees of impermeability of which one of ordinary skill in the art recognizes as having a potential benefit. In this regard, the antagonist-impermeable material substantially prevents or prevents the release of the antagonist to an extent that at least about 80% of the antagonist is prevented from being released from the sequestering subunit in the gastrointestinal tract for a time period that is greater than 24 hours. Preferably, the antagonist-impermeable material prevents release of at least about 90% of the antagonist from the sequestering subunit in the gastrointestinal tract for a time period that is greater than 24 hours. More preferably, the antagonist-impermeable material prevents release of at least about 95% of the antagonist from the sequestering subunit. Most preferably, the antagonist-impermeable material prevents release of at least about 99% of the antagonist from the sequestering subunit in the gastrointestinal tract for a time period that is greater than 24 hours. The antagonist-impermeable material prevents or substantially prevents the release of die antagonist in the gastrointestinal tract for a time period that is greater than 24 hours, and desirably, at least about 48 hours. More desirably, the antagonist-impermeable material prevents or substantially prevents the release of the aversive agent from the sequestering subunit for a time period of at least about 72 hours.

Preferably, the first antagonist-impermeable material comprises a hydrophobic material, such that the antagonist is not released or substantially not released during its transit through the gastrointestinal tract when administered orally as intended, without having been tampered with. Suitable hydrophobic materials for use in the invention are described herein and set forth below. The hydrophobic material is preferably a pharmaceutically acceptable hydrophobic material. Preferably, the pharmaceutically acceptable hydrophobic material comprises a cellulose polymer.

It is preferred that the first antagonist-impermeable material comprises a polymer insoluble in the gastrointestinal tract. One of ordinary skill in the art appreciates that a polymer that is insoluble in the gastrointestinal tract will prevent the release of the antagonist upon ingestion of the sequestering subunit. The polymer can be a cellulose or an acrylic polymer. Desirably, the cellulose is selected from the group consisting of ethylcellulose, cellulose acetate, cellulose propionate, cellulose acetate propionate, cellulose acetate butyrate, cellulose acetate phthalate, cellulose triacetate, and combinations thereof. Ethylcellulose includes, for example, one that has an ethoxy content of about 44 to about 55%. Ethylcellulose can be used in the form of an aqueous dispersion, an alcoholic solution, or a solution in other suitable solvents. The cellulose can have a degree of substitution (D.S.) on the anhydroglucose unit, from greater than zero and up to 3 inclusive. By “degree of substitution” is meant the average number of hydroxyl groups on the anhydroglucose unit of the cellulose polymer that are replaced by a substituting group. Representative materials include a polymer selected from the group consisting of cellulose acylate, cellulose diacylate, cellulose triacylate, cellulose acetate, cellulose diacetate, cellulose triacetate, monocellulose alkenylate, dicellulose alkenylate, tricellulose alkenylate, monocellulose alkenylates, dicellulose alkenylates, tricellulose alkenylates, monocellulose aroylates, dicellulose aroylates, and tricellulose aroylates.

More specific celluloses include cellulose propionate having a D.S. of 1.8 and a propyl content of 39.2 to 45 and a hydroxy content of 2.8 to 5.4%; cellulose acetate butyrate having a D.S. of 1.8, an acetyl content of 13 to 15% and a butyryl content of 34 to 39%; cellulose acetate butyrate having an acetyl content of 2 to 29%, a butyryl content of 17 to 53% and a hydroxy content of 0.5 to 4.7%; cellulose triacylate having a D.S. of 2.9 to 3, such as cellulose triacetate, cellulose trivalerate, cellulose trilaurate, cellulose tripalmitate, cellulose trisuccinate, and cellulose trioctanoate; cellulose diacrylates having a D.S. of 2.2 to 2.6, such as cellulose disuccinate, cellulose dipalmitate, cellulose dioctanoate, cellulose dipentanoate, and coesters of cellulose, such as cellulose acetate butyrate, cellulose acetate octanoate butyrate, and cellulose acetate propionate.

Additional cellulose polymers useful for preparing a sequestering subunit of the invention includes acetaldehyde dimethyl cellulose acetate, cellulose acetate ethylcarbamate, cellulose acetate methylcarbamate, and cellulose acetate dimethylaminocellulose acetate.

The acrylic polymer preferably is selected from the group consisting of methacrylic polymers, acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), glycidyl methacrylate copolymers, and combinations thereof. An acrylic polymer useful for preparation of a sequestering subunit of the invention includes acrylic resins comprising copolymers synthesized from acrylic and methacylic acid esters (e.g., the copolymer of acrylic acid lower alkyl ester and methacrylic acid lower alkyl ester) containing about 0.02 to about 0.03 mole of a tri (lower alkyl) ammonium group per mole of the acrylic and methacrylic monomer used. An example of a suitable acrylic resin is ammonio methacrylate copolymer NF21, a polymer manufactured by Rohm Pharma GmbH, Darmstadt, Germany, and sold under the Eudragit® trademark. Eudragit RS30D is preferred. Eudragit® is a water-insoluble copolymer of ethyl acrylate (EA), methyl methacrylate (MM) and trimethylammoniumethyl methacrylate chloride (TAM) in which the molar ratio of TAM to the remaining components (EA and MM) is 1:40. Acrylic resins, such as Eudragit®, can be used in the form of an aqueous dispersion or as a solution in suitable solvents.

In another preferred embodiment, the antagonist-impermeable material is selected from the group consisting of polylactic acid, polyglycolic acid, a co-polymer of polylactic acid and polyglycolic acid, and combinations thereof. In certain other embodiments, the hydrophobic material includes a biodegradable polymer comprising a poly(lactic/glycolic acid) (“PLGA”), a polylactide, a polyglycolide, a polyanhydride, a polyorthoester, polycaprolactones, polyphosphazenes, polysaccharides, proteinaceous polymers, polyesters, polydioxanone, polygluconate, poly lactic-acid-polyethylene oxide copolymers, poly(hydroxybutyrate), polyphosphoester or combinations thereof.

Preferably, the biodegradable polymer comprises a poly(lactic/glycolic acid), a copolymer of lactic and glycolic acid, having a molecular weight of about 2,000 to about 500,000 daltons. The ratio of lactic acid to glycolic acid is preferably from about 100:1 to about 25:75, with the ratio of lactic acid to glycolic acid of about 65:35 being more preferred.

Poly(lactic/glycolic acid) can be prepared by the procedures set forth in U.S. Pat. No. 4,293,539 (Ludwig et al.), which is incorporated herein by reference. In brief, Ludwig prepares the copolymer by condensation of lactic acid and glycolic acid in the presence of a readily removable polymerization catalyst (e.g., a strong ion-exchange resin such as Dowex HCR-W2-H). The amount of catalyst is not critical to the polymerization, but typically is from about 0.01 to about 20 parts by weight relative to the total weight of combined lactic acid and glycolic acid. The polymerization reaction can be conducted without solvents at a temperature from about 100° C. to about 250° C. for about 48 to about 96 hours, preferably under a reduced pressure to facilitate removal of water and by-products. Poly(lactic/glycolic acid) is then recovered by filtering the molten reaction mixture in an organic solvent, such as dichloromethane or acetone, and then filtering to remove the catalyst.

Suitable plasticizers, for example, acetyl triethyl citrate, acetyl tributyl citrate, triethyl citrate, diethyl phthalate, dibutyl phthalate, or dibutyl sebacate, also can be admixed with the polymer used to make the sequestering subunit. Additives, such as coloring agents, talc and/or magnesium stearate, and other additives also can be used in making the present inventive sequestering subunit.

In certain embodiments, additives may be included in the compositions to improve the sequestering characteristics of the sequestering subunit. As described below, the ratio of additives or components with respect to other additives or components may be modified to enhance or delay improve sequestration of the agent contained within the subunit. Various amounts of a functional additive (i.e., a charge-neutralizing additive) may be included to vary the release of an antagonist, particularly where a water-soluble core (i.e., a sugar sphere) is utilized. For instance, it has been determined that the inclusion of a low amount of charge-neutralizing additive relative to sequestering polymer on a weight-by-weight basis may cause decreased release of the antagonist.

In certain embodiments, a surfactant may serve as a charge-neutralizing additive. Such neutralization may in certain embodiments reduce the swelling of the sequestering polymer by hydration of positively charged groups contained therein. Surfactants (ionic or non-ionic) may also be used in preparing the sequestering subunit. It is preferred that the surfactant be ionic. Suitable exemplary agents include, for example, alkylaryl sulphonates, alcohol sulphates, sulphosuccinates, sulphosuccinamates, sarcosinates or taurates and others. Additional examples include but are not limited to ethoxylated castor oil, benzalkonium chloride, polyglycolyzed glycerides, acetylated monoglycerides, sorbitan fatty acid esters, poloxamers, polyoxyethylene fatty acid esters, polyoxyethylene derivatives, monoglycerides or ethoxylated derivatives thereof, diglycerides or polyoxyethylene derivatives thereof, sodium docusate, sodium lauryl sulfate, dioctyl sodium sulphosuccinate, sodium lauryl sarcosinate and sodium methyl cocoyl taurate, magnesium lauryl sulfate, triethanolamine, cetrimide, sucrose laurate and other sucrose esters, glucose (dextrose) esters, simethicone, ocoxynol, dioctyl sodiumsulfosuccinate, polyglycolyzed glycerides, sodiumdodecylbenzene sulfonate, dialkyl sodiumsulfosuccinate, fatty alcohols such as lauryl, cetyl, and steryl, glycerylesters, cholic acid or derivatives thereof, lecithins, and phospholipids. These agents are typically characterized as ionic (i.e., anionic or cationic) or nonionic. In certain embodiments described herein, an anionic surfactant such as sodium lauyl sulfate (SLS) is preferably used (U.S. Pat. No. 5,725,883, U.S. Pat. No. 7,201,920; EP 502642A1; Shokri, et al. Pharm. Sci. 2003. The effect of sodium lauryl sulphate on the release of diazepam from solid dispersions prepared by cogrinding technique. Wells, et al. Effect of Anionic Surfactants on the Release of Chlorpheniramine Maleate From an Inert, Heterogeneous Matrix, Drug Development and Industrial Pharmacy 18(2) (1992): 175-186. Rao, et al. “Effect of Sodium Lauryl Sulfate on the Release of Rifampicin from Guar Gum Matrix.” Indian Journal of Pharmaceutical Science (2000): 404-406; Knop, et al. Influence of surfactants of different charge and concentration on drug release from pellets coated with an aqueous dispersion of quaternary acrylic polymers, STP Pharma Sciences. Vol. 7, No. 6, (1997) 507-512). Other suitable agents are known in the art.

As shown herein, SLS is particularly useful in combination with Eudragit RS when the sequestering subunit is built upon a sugar sphere substrate. The inclusion of SLS at less than approximately 6.3% on a weight-to-weight basis relative to the sequestering polymer (i.e., Eudragit RS) may provide a charge neutralizing function (theoretically 20% and 41% neutralization, respectively), and thereby significantly slow the release of the active agent encapsulated thereby (i.e., the antagonist naltrexone). Inclusion of more than approximately 6.3% SLS relative to the sequestering polymer appears to increase release of the antagonist from the sequestering subunit. With respect to SLS used in conjunction with Eudragit® RS, it is preferred that the SLS is present at approximately 1%, 2%, 3%, 4% or 5%, and typically less than 6% on a w/w basis relative to the sequestering polymer (i.e., Eudragit® RS). In preferred embodiments, SLS may be present at approximately 1.6% or approximately 3.3% relative to the sequestering polymer. As discussed above, many agents (i.e., surfactants) may substitute for SLS in the compositions disclosed herein.

Additionally useful agents include those that may physically block migration of the antagonist from the subunit and/or enhance the hydrophobicity of the barrier. One exemplary agent is talc, which is commonly used in pharmaceutical compositions (Pawar et al. Agglomeration of Ibuprofen With Talc by Novel Crystallo-Co-Agglomeration Technique, AAPS PharmSciTech. 2004; 5(4): article 55). As shown in the Examples, talc is especially useful where the sequestering subunit is built upon a sugar sphere core. Any form of talc may be used, so long as it does not detrimentally affect the function of the composition. Most talc results from the alteration of dolomite (CaMg(CO₃)² or magnesite (MgO) in the presence of excess dissolved silica (SiO₂) or by altering serpentine or quartzite. Talc may be include minerals such as tremolite (CaMg₃(SiO₃)₄), serpentine (3MgO.2SiO₃.2H₂O), andiophyllite (Mg₇.(OH)₂.(Si₄O₁₁)₂), magnesite, mica, chlorite, dolomite, the calcite form of calcium carbonate (CaCO₃), iron oxide, carbon, quartz, and/or manganese oxide. The presence of such impurities may be acceptable in the compositions described herein provided the function of the talc is maintained. It is preferred that that talc be USP grade. As mentioned above, the function of talc as described herein is to enhance the hydrophobicity and therefore the functionality of the sequestering polymer. Many substitutes for talc may be utilized in the compositions described herein as may be determined by one of skill in the art.

It has been determined that the ratio of talc to sequestering polymer may make a dramatic difference in the functionality of the compositions described herein. For instance, the Examples described below demonstrate that the talc to sequestering polymer ratio (w/w) is important with respect to compositions designed to prevent the release of naltrexone therefrom. It is shown therein that inclusion of an approximately equivalent amount (on a weight-by-weight basis) of talc and Eudragit® RS results in a very low naltrexone release profile. In contrast, significantly, lower or higher both a lower (69% w/w) and a higher (151% w/w) talc:Eudragit® RS ratios result in increased release of naltrexone release. Thus, where talc and Eudragit® RS are utilized, it is preferred that talc is present at approximately 75%, 80%, 85%, 90%, 95%, 100%, 105%, 110%, 115%, 120% or 125% w/w relative to Eudragit® RS. As described above, the most beneficial ratio for other additives or components will vary and may be determined using standard experimental procedures.

In certain embodiments, such as where a water-soluble core is utilized, it is useful to include agents that may affect the osmotic pressure of the composition (i.e., an osmotic pressure regulating agent) (see, in general, WO 2005/046561A2 and WO 2005/046649 A2 relating to Eudramode®). This agent is preferably applied to the Eudragit® RS/talc layer described above. In a pharmaceutical unit comprising a sequestering subunit overlayed by an active agent (i.e., a controlled-release agonist preparation), the osmotic pressure regulating agent is preferably positioned immediately beneath the active agent layer. Suitable osmotic pressure regulating agents may include, for instance, hydroxypropylmethyl cellulose (HPMC) or chloride ions (i.e., from NaCl), or a combination of HPMC and chloride ions (i.e., from NaCl). Other ions that may be useful include bromide or iodide. The combination of sodium chloride and HPMC may be prepared in water or in a mixture of ethanol and water, for instance. HPMC is commonly utilized in pharmaceutical compositions (see, for example. U.S. Pat. Nos. 7,226,620 and 7,229,982). In certain embodiments, HPMC may have a molecular weight ranging from about 10,000 to about 1,500,000, and typically from about 5000 to about 10,000 (low molecular weight HPMC). The specific gravity of HPMC is typically from about 1.19 to about 1.31, with an average specific gravity of about 1.26 and a viscosity of about 3600 to 5600. HPMC may be a water-soluble synthetic polymer. Examples of suitable, commercially available hydroxypropyl methylcellulose polymers include Methocel K100 LV and Methocel K4M (Dow). Other HPMC additives are known in the art and may be suitable in preparing the compositions described herein. As shown in the Examples, the inclusion of NaCl (with HPMC) was found to have positively affect sequestration of naltrexone by Eudragit® RS. In certain embodiments, it is preferred that the charge-neutralizing additive (i.e., NaCl) is included at less than approximately 1, 2, 3, 4, 5, 6, 7, 8, 9, or 10% of the composition on a weight-by-weight basis. In other preferred embodiments, the charge-neutralizing additive is present at approximately 4% of the composition on a weight-by-weight basis.

Thus, in one embodiment, a sequestering subunit built upon a sugar sphere substrate is provided comprising a sequestering polymer (i.e., Eudragit® RS) in combination with several optimizing agents, including sodium lauryl sulfate (SLS) as a charge-neutralizing agent to reduce swelling of the film by hydration of the positively charged groups on the polymer; talc to create a solid impermeable obstacle to naltrexone transport through the film and as a hydrophobicity-enhancing agent; and a chloride ion (i.e., as NaCl) as an osmotic pressure reducing agent. The ratio of each of the additional ingredients relative to the sequestering polymer was surprisingly found to be important to the function of the sequestering subunit. For instance, the Examples provide a sequestering subunit including a sequestering polymer and the optimizing agents SLS at less than 6%, preferably 1-4%, and even more preferably 1.6% or 3.3% on a w/w basis relative to Eudragit RS; talc in an amount approximately equal to Eudragit® RS (on a w/w basis); and, NaCl present at approximately 4% on a w/w basis relative to Eudragit® RS.

The therapeutic agent applied upon the sequestering subunit may be any medicament. The therapeutic agent of the present inventive compositions can be any medicinal agent used for the treatment of a condition or disease, a pharmaceutically acceptable salt thereof, or an analogue of either of the foregoing. The therapeutic agent can be, for example, an analgesic (e.g., an opioid agonist, aspirin, acetaminophen, nonsteroidal anti-inflammatory drugs (“NSAIDS”), N-methyl-D-aspartate (“NMDA”) receptor antagonists, cyclooxygenase-II inhibitors (“COX-II inhibitors”), and glycine receptor antagonists), an antibacterial agent, an and viral agent, an anti-microbial agent, anti-infective agent, a chemotherapeutic, an immunosuppressant agent, an antiitussive, an expectorant, a decongestant, an antihistamine drugs, a decongestant, antihistamine drugs, and the like. Preferably, the therapeutic agent is one that is addictive (physically and/or psychologically) upon repeated use and typically leads to abuse of the therapeutic agent. In this regard, the therapeutic agent can be any opioid agonist as discussed herein.

The therapeutic agent can be an opioid agonist. By “opioid” is meant to include a drug, hormone, or other chemical or biological substance, natural or synthetic, having a sedative, narcotic, or otherwise similar effect(s) to those containing opium or its natural or synthetic derivatives. By “opioid agonist,” sometimes used herein interchangeably with terms “opioid” and “opioid analgesic,” is meant to include one or more opioid agonists, either alone or in combination, and is further meant to include the base of the opioid, mixed or combined agonist-antagonists, partial agonists, pharmaceutically acceptable salts thereof, stereoisomers thereof, ethers thereof, esters thereof, and combinations thereof.

Opioid agonists include, for example, alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, dihydrocodeine, dihydroetorphine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenazocine, phenomorphan, phenoperidine, piminodine, piritramide, propheptazine, promedol, properidine, propiram, propoxyphene, sufentanil, tramadol, tilidine, derivatives or complexes thereof, pharmaceutically acceptable salts thereof, and combinations thereof. Preferably, the opioid agonist is selected from the group consisting of hydrocodone, hydromorphone, oxycodone, dihydrocodeine, codeine, dihydromorphine, morphine, buprenorphine, derivatives or complexes thereof, pharmaceutically acceptable salts thereof, and combinations thereof. Most preferably, the opioid agonist is morphine, hydromorphone, oxycodone or hydrocodone. In a preferred embodiment, the opioid agonist comprises oxycodone or hydrocodone and is present in the dosage form in an amount of about 15 to about 45 mg, and the opioid antagonist comprises naltrexone and is present in the dosage form in an amount of about 0.5 to about 5 mg.

Equianalgesic doses of these opioids, in comparison to a 15 mg dose of hydrocodone, are set forth in Table 1 below:

TABLE 1
Equianalgesic Doses of Opioids

| Opioid | Calculated Dose (mg) |
|---|---|
| Oxycodone | 13.5 |
| Codeine | 90.0 |
| Hydrocodone | 15.0 |
| Hydromorphone | 3.375 |
| Levorphanol | 1.8 |
| Meperidine | 135.0 |
| Methadone | 9.0 |
| Morphine | 27.0 |

Hydrocodone is a semisynthetic narcotic analgesic and antiitussive with multiple nervous system and gastrointestinal actions. Chemically, hydrocodone is 4,5-epoxy-3-methoxy-17-methylmorphinan-6-one, and is also known as dihydrocodeinone. Like other opioids, hydrocodone can be habit-forming and can produce drug dependence of the morphine type. Like other opium derivatives, excess doses of hydrocodone will depress respiration.

Oral hydrocodone is also available in Europe (e.g., Belgium, Germany, Greece, Italy. Luxembourg Norway and Switzerland) as an antiitussive agent. A parenteral formulation is also available in Germany as an antiitussive agent. For use as an analgesic, hydrocodone bitartrate is commonly available in the United States only as a fixed combination with non-opiate drugs (e.g., ibuprofen, acetaminophen, aspirin; etc.) for relief of moderate to moderately severe pain.

A common dosage form of hydrocodone is in combination with acetaminophen and is commercially available, for example, as Lortab® in the United States from UCB Pharma, Inc. (Brussels, Belgium), as 2.5/500 mg, 5/500 mg, 7.5/500 mg and 10/500 mg Hydrocodone/acetaminophen tablets. Tablets are also available in the ratio of 7.5 mg hydrocodone bitartrate and 650 mg acetaminophen and a 7.5 mg hydrocodone bitartrate and 750 mg acetaminophen. Hydrocodone, in combination with aspirin, is given in an oral dosage form to adults generally in 1-2 tablets every 4-6 hours as needed to alleviate pain. The tablet form is 5 mg hydrocodone bitartrate and 224 mg aspirin with 32 mg caffeine; or 5 mg hydrocodone bitartrate and 500 mg aspirin. Another formulation comprises hydrocodone bitartrate and ibuprofen. Vicoprofen®, commercially available in the U.S. from Knoll Laboratories (Mount Olive, N.J.), is a tablet containing 7.5 mg hydrocodone bitartrate and 200 mg ibuprofen. The invention is contemplated to encompass all such formulations, with the inclusion of the opioid antagonist and/or antagonist in sequestered form as part of a subunit comprising an opioid agonist.

Oxycodone, chemically known as 4,5-epoxy-14-hydroxy-3-methoxy-17-methylmorphinan-6-one, is an opioid agonist whose principal therapeutic action is analgesia. Other therapeutic effects of oxycodone include anxiolysis, euphoria and feelings of relaxation. The precise mechanism of its analgesic action is not known, but specific CNS opioid receptors for endogenous compounds with opioid-like activity have been identified throughout the brain and spinal cord and play a role in the analgesic effects of this drug.

Oxycodone is commercially available in the United States, e.g., as Oxycotin® from Purdue Pharma L.P. (Stamford, Conn.), as controlled-release tablets for oral administration containing 10 mg, 20 mg, 40 mg or 80 mg oxycodone hydrochloride, and as OxyIR™, also from Purdue Pharma L.P., as immediate-release capsules containing 5 mg oxycodone hydrochloride. The invention is contemplated to encompass all such formulations, with the inclusion of an opioid antagonist and/or antagonist in sequestered form as part of a subunit comprising an opioid agonist.

Oral hydromorphone is commercially available in the United States, e.g., as Dilaudid® from Abbott Laboratories (Chicago, Ill.). Oral morphine is commercially available in the United States, e.g., as Kadian® from Faulding Laboratories (Piscataway, N.J.).

Exemplary NSAIDS include ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zidometacin, acemetacin, fentiazac, clidanac, oxpinac, mefenamic acid, meclofenamic acid, flufenamic acid, niflumic acid, tolfenamic acid, diflunisal, flufenisal, piroxicam, sudoxican or isoxicam, and the like. Useful dosages of these drugs are well-known.

Exemplary NMDA receptor medicaments include morphinans, such as dextromethorphan or dextrophan, ketamine, d-methadone, and pharmaceutically acceptable salts thereof, and encompass drugs that block a major intracellular consequence of NMDA-receptor activation, e.g., a ganglioside, such as (6-aminothexyl)-5-chloro-1-naphthalenesulfonamide. These drugs are stated to inhibit the development of tolerance to and/or dependence on addictive drugs, e.g., narcotic analgesics, such as morphine, codeine; etc., in U.S. Pat. Nos. 5,321,012 and 5,556,838 (both to Mayer et al.), both of which are incorporated herein by reference, and to treat chronic pain in U.S. Pat. No. 5,502,058 (Mayer et al.), incorporated herein by reference. The NMDA agonist can be included alone or in combination with a local anesthetic, such as lidocaine, as described in these patents by Mayer et al.

COX-2 inhibitors have been reported in the art, and many chemical compounds are known to produce inhibition of cyclooxygenase-2. COX-2 inhibitors are described, for example, in U.S. Pat. Nos. 5,616,601; 5,604,260; 5,593,994; 5,550,142; 5,536,752; 5,521,213; 5,475,995; 5,639,780; 5,604,253; 3,552,422; 5,510,368; 5,436,265; 5,409,944 and 5,130,311, all of which are incorporated herein by reference. Certain preferred COX-2 inhibitors include celecoxib (SC-58635), DUP-697, flosulide (CGP-28238), meloxicam, 6-methoxy-2-naphthylacetic acid (6-NMA), MK-966 (also known as Vioxx), nabumetone (prodrug for 6-MNA), nimesulide. NS-398, SC-5766, SC-58215, T-614, or combinations thereof. Dosage levels of COX-2 inhibitor on the order of from about 0.005 mg to about 140 mg per kilogram of body weight per day have been shown to be therapeutically effective in combination with an opioid analgesic. Alternatively, about 0.25 mg to about 7 g per patient per day of a COX-2 inhibitor can be administered in combination with an opioid analgesic.

The treatment of chronic pain via the use of glycine receptor antagonists and the identification of such drugs is described in U.S. Pat. No. 5,514,680 (Weber et al.), which is incorporated herein by reference.

Pharmaceutically acceptable salts of the antagonist or agonist agents discussed herein include metal salts, such as sodium salt, potassium salt, cesium salt, and the like; alkaline earth metals, such as calcium salt, magnesium salt, and the like; organic amine salts, such as triethylamine salt, pyridine salt, picoline salt, ethanolamine salt, triethanolamine salt, dicyclohexylamine salt. N,N′-dibenzylethylenediamine salt, and the like; inorganic acid salts, such as hydrochloride, hydrobromide, sulfate, phosphate, and the like; organic acid salts, such as formate, acetate, trifluoroacetate, maleate, taurate, and the like; sulfonates, such as methanesulfonate, benzenesulfonate, p-toluenesulfonate, and the like; amino acid salts, such as arginate, asparaginate, glutamate, and the like.

In embodiments in which the opioid agonist comprises hydrocodone, the sustained-release oral dosage forms can include analgesic doses from about 8 mg to about 50 mg of hydrocodone per dosage unit. In sustained-release oral dosage forms where hydromorphone is the therapeutically active opioid, it is included in an amount from about 2 mg to about 64 my hydromorphone hydrochloride. In another embodiment, the opioid agonist comprises morphine, and the sustained-release oral dosage forms of the invention include from about 2.5 mg to about 800 mg morphine, by weight. In yet another embodiment, the opioid agonist comprises oxycodone and the sustained-release oral dosage forms include from about 2.5 mg to about 800 mg oxycodone. In certain preferred embodiments, the sustained-release oral dosage forms include from about 20 mg to about 30 mg oxycodone. Controlled release oxycodone formulations are known in the art. The following documents describe various controlled-release oxycodone formulations suitable for use in the invention described herein, and processes for their manufacture: U.S. Pat. Nos. 5,266,331; 5,549,912; 5,508,042; and 5,656,295, which are incorporated herein by reference. The opioid agonist can comprise tramadol and the sustained-release oral dosage forms can include from about 25 mg to 800 mg tramadol per dosage unit.

Methods of making any of the sequestering subunits of the invention are known in the art. See, for example, Remnington: The Science and Practice of Pharmacy, Alfonso R. Genaro (ed), 20^{th} edition, and Example 2 set forth below. The sequestering subunits cant be prepared by any suitable method to provide, for example, beads, pellets, granules, spheroids, and the like. Spheroids or beads, coated with an active ingredient can be prepared, for example, by dissolving the active ingredient in water and then spraying the solution onto a substrate, for example, nu pariel 18/20 beads, using a Wurster insert. Optionally, additional ingredients are also added prior to coating the beads in order to assist the active ingredient in binding to the substrates, and/or to color the solution; etc. The resulting substrate-active material optionally can be overcoated with a barrier material to separate the therapeutically active agent from the next coat of material, e.g., release-retarding material. Preferably, the barrier material is a material comprising hydroxypropyl methylcellulose. However, any film-former known in the art can be used. Preferably, the barrier material does not affect the dissolution rate of the final product.

Pellets comprising an active ingredient can be prepared, for example, by a melt pelletization technique. Typical of such techniques is when the active ingredient in finely divided form is combined with a binder (also in particulate form) and other optional inert ingredients, and thereafter the mixture is pelletized, e.g., by mechanically working the mixture in a high shear mixer to form the pellets (e.g., pellets, granules, spheres, beads; etc., collectively referred to herein as “pellets”). Thereafter, the pellets can be sieved in order to obtain pellets of the requisite size. The binder material is preferably in particulate form and has a melting point above about 40° C. Suitable binder substances include, for example, hydrogenated castor oil, hydrogenated vegetable oil, other hydrogenated fats, fatty alcohols, fatty acid esters, fatty acid glycerides, and the like.

The diameter of the extruder aperture or exit port also can be adjusted to vary the thickness of the extruded strands. Furthermore, the exit part of the extruder need not be round; it can be oblong, rectangular; etc. The exiting strands can be reduced to particles using a hot wire cutter, guillotine; etc.

The melt-extruded multiparticulate system can be, for example, in the form of granules, spheroids, pellets, or the like, depending upon the extruder exit orifice. The terms “melt-extruded multiparticulate(s)” and “melt-extruded multiparticulate system(s)” and “melt-extruded particles” are used interchangeably herein and include a plurality of subunits, preferably within a range of similar size and/or shape. The melt-extruded multiparticulates are preferably in a range of from about 0.1 to about 12 mm in length and have a diameter of from about 0.1 to about 5 mm. In addition, the melt-extruded multiparticulates can be any geometrical shape within this size range. Alternatively, the extrudate can simply be cut into desired lengths and divided into unit doses of the therapeutically active agent without the need of a spheronization step.

The substrate also can be prepared via a granulation technique. Generally, melt-granulation techniques involve melting a normally solid hydrophobic material, e.g., a wax, and incorporating an active ingredient therein. To obtain a sustained-release dosage form, it can be necessary to incorporate an additional hydrophobic material.

A coating composition can be applied onto a substrate by spraying it onto the substrate using any suitable spray equipment. For example, a Wurster fluidized-bed system can be used in which an air flow from underneath, fluidizes the coated material and effects drying, while the insoluble polymer coating is sprayed on. The thickness of the coating will depend on the characteristics of the particular coating composition, and can be determined by using routine experimentation.

Any manner of preparing a subunit can be employed. By way of example, a subunit in the form of a pellet or the like can be prepared by co-extruding a material comprising the opioid agonist and a material comprising the opioid antagonist and/or antagonist in sequestered form. Optionally, the opioid agonist composition can cover, e.g., overcoat, the material comprising the antagonist and/or antagonist in sequestered form. A bead, for example, can be prepared by coating a substrate comprising an opioid antagonist and/or an antagonist in sequestered form with a solution comprising an opioid agonist.

The sequestering subunits of the invention are particularly well-suited for use in compositions comprising the sequestering subunit and a therapeutic agent in releasable form. In this regard, the invention also provides a composition comprising any of the sequestering subunits of the invention and a therapeutic agent in releasable form. By “releasable form” is meant to include immediate release, intermediate release, and sustained-release forms. The therapeutic agent can be formulated to provide immediate release of the therapeutic agent. In preferred embodiments, the composition provides sustained-release of the therapeutic agent.

The therapeutic agent in sustained-release form is preferably a particle of therapeutic agent that is combined with a release-retarding material. The release-retarding material is preferably a material that permits release of the therapeutic agent at a sustained rate in an aqueous medium. The release-retarding material can be selectively chosen so as to achieve, in combination with the other stated properties, a desired in vitro release rate.

In a preferred embodiment, the oral dosage form of the invention can be formulated to provide for an increased duration of therapeutic action allowing once-daily dosing. In general, a release-retarding material is used to provide the increased duration of therapeutic action. Preferably, the once-daily dosing is provided by the dosage forms and methods described in U.S. Patent Application Pub. No. 2005/0020613 to Boehm, entitled “Sustained-Release Opioid Formulations and Method of Use,” filed on Sep. 22, 2003, and incorporated herein by reference.

Preferred release-retarding materials include acrylic polymers, alkylcelluloses, shellac, rein, hydrogenated vegetable oil, hydrogenated castor oil, and combinations thereof. In certain preferred embodiments, the release-retarding material is a pharmaceutically acceptable acrylic polymer, including acrylic acid and methacrylic acid copolymers, methyl methacrylate copolymers, ethoxyethyl methacrylates, cynaoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, and glycidyl methacrylate copolymers. In certain preferred embodiments, the acrylic polymer comprises one or more ammonio methacrylate copolymers. Ammonio methacrylate copolymers are well-known in the art, and are described in NF21, the 21^{st} edition of the National Formulary, published by the United States Pharmacopeial Convention Inc. (Rockville, Md.), as fully polymerized copolymers of acrylic and methacrylic acid esters with a low content of quaternary ammonium groups. In other preferred embodiments, the release-retarding material is an alkyl cellulosic material, such as ethylcellulose. Those skilled in the art will appreciate that other cellulosic polymers, including other alkyl cellulosic polymers, can be substituted for part or all of the ethylcellulose.

Release-modifying agents, which affect the release properties of the release-retarding material, also can be used. In a preferred embodiment, the release-modifying agent functions as a pore-former. The pore-former can be organic or inorganic, and include materials that can be dissolved, extracted or leached from the coating in the environment of use. The pore-former can comprise one or more hydrophilic polymers, such as hydroxypropylmethylcellulose. In certain preferred embodiments, the release-modifying agent is selected from hydroxypropylmethylcellulose, lactose, metal stearates, and combinations thereof.

The release-retarding material can also include an erosion-promoting agent, such as starch and gums; a release-modifying agent useful for making microporous lamina in the environment of use, such as polycarbonates comprised of linear polyesters of carbonic acid in which carbonate groups reoccur in the polymer chain; and/or a semi-permeable polymer.

The release-retarding material can also include an exit means comprising at least one passageway, orifice, or the like. The passageway can be formed by such methods as those disclosed in U.S. Pat. Nos. 3,845,770; 3,916,889; 4,063,064; and 4,088,864, which are incorporated herein by reference. The passageway can have any shape, such as round, triangular, square, elliptical, irregular, etc.

In certain embodiments, the therapeutic agent in sustained-release form can include a plurality of substrates comprising the active ingredient, which substrates are coated with a sustained-release coating comprising a release-retarding material.

The sustained-release preparations of the invention can be made in conjunction with any multiparticulate system, such as beads, ion-exchange resin beads, spheroids, microspheres, seeds, pellets, granules, and other multiparticulate systems in order to obtain a desired sustained-release of the therapeutic agent. The multiparticulate system can be presented in a capsule or in any other suitable unit dosage form.

In certain preferred embodiments, more than one multiparticulate system can be used, each exhibiting different characteristics, such as pH dependence of release, time for release in various media (e.g., acid, base, simulated intestinal fluid), release in vivo, size and composition.

To obtain a sustained-release of the therapeutic agent in a manner sufficient to provide a therapeutic effect for the sustained durations, the therapeutic agent can be coated with an amount of release-retarding material sufficient to obtain a weight gain level from about 2 to about 30%, although die coat can be greater or lesser depending upon the physical properties of the particular therapeutic agent utilized and the desired release rate, among other things. Moreover, there can be more than one release-retarding material used in the coat, as well as various other pharmaceutical excipients.

Solvents typically used for the release-retarding material include pharmaceutically acceptable solvents, such as water, methanol, ethanol, methylene chloride and combinations thereof.

In certain embodiments of the invention, the release-retarding material is in the form of a coating comprising an aqueous dispersion of a hydrophobic polymer. The inclusion of an effective amount of a plasticizer in the aqueous dispersion of hydrophobic polymer will further improve the physical properties of the film. For example, because ethylcellulose has a relatively high glass transition temperature and does not form flexible films under normal coating conditions, it is necessary to plasticize the ethylcellulose before using the same as a coating material. Generally, the amount of plasticizer included in a coating solution is based on the concentration of the film-former, e.g., most often from about 1 to about 50 percent by weight of the film-former. Concentrations of the plasticizer, however, can be determined by routine experimentation.

Examples of plasticizers for ethylcellulose and other celluloses include dibutyl sebacate, diethyl phthalate, triethyl citrate, tributyl citrate, and triacetin, although it is possible that other plasticizers (such as acetylated monoglycerides, phthalate esters, castor oil; etc.) can be used.

Examples of plasticizers for the acrylic polymers include citric acid esters, such as triethyl citrate NF21, tributyl citrate, dibutyl phthalate, and possibly 1,2-propylene glycol, polyethylene glycols, propylene glycol, diethyl phthalate, castor oil, and triacetin, although it is possible that other plasticizers (such as acetylated monoglycerides, phthalate esters, castor oil; etc.) can be used.

The sustained-release profile of drug release in the formulations of the invention (either in vivo or in vitro) can be altered, for example, by using more than one release-retarding material, varying the thickness of the release-retarding material, changing the particular release-retarding material used, altering the relative amounts of release-retarding material, altering the manner in which the plasticizer is added (e.g., when the sustained-release coating is derived from an aqueous dispersion of hydrophobic polymer), by varying the amount of plasticizer relative to retardant material, by the inclusion of additional ingredients or excipients, by altering the method of manufacture; etc.

In certain other embodiments, the oral dosage form can utilize a multiparticulate sustained-release matrix. In certain embodiments, the sustained-release matrix comprises a hydrophilic and/or hydrophobic polymer, such as gums, cellulose ethers, acrylic resins and protein-derived materials. Of these polymers, the cellulose ethers, specifically hydroxyalkylcelluloses and carboxyalkylcelluloses, are preferred. The oral dosage form can contain between about 1% and about 80% (by weight) of at least one hydrophilic or hydrophobic polymer.

The hydrophobic material is preferably selected from the group consisting of alkylcellulose, acrylic and methacrylic acid polymers and copolymers, shellac, zein, hydrogenated castor oil, hydrogenated vegetable oil, or mixtures thereof. Preferably, the hydrophobic material is a pharmaceutically acceptable acrylic polymer, including acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylicacid), poly(methacrylic acid), methacrylic acid alkylamine copolymer, poly(methyl methacrylate), poly(methacrylic acid) (anhydride), polymethacrylate, polyacrylamide, poly(methacrylic acid anhydride), and glycidyl methacrylate copolymers. In other embodiments, the hydrophobic material can also include hydrooxyalkylcelluloses such as hydroxypropylmethylcellulose and mixtures of the foregoing.

Preferred hydrophobic materials are water-insoluble with more or less pronounced hydrophobic trends. Preferably, the hydrophobic material has a melting point from about 30° C. to about 20° C., more preferably from about 45° C. to about 90° C. The hydrophobic material can include neutral or synthetic waxes, fatty alcohols (such as lauryl, myristyl, stearyl, cetyl or preferably cetostearyl alcohol), tatty acids, including fatty acid esters, fatty acid glycerides (mono-, di-, and tri-glycerides), hydrogenated fats, hydrocarbons, normal waxes, stearic acid, stearyl alcohol and hydrophobic and hydrophilic materials having hydrocarbon backbones. Suitable waxes include beeswax, glycowax, castor wax, carnauba wax and wax-like substances, e.g., material normally solid at room temperature and having a melting point of from about 30° C. to about 100° C.

Preferably, a combination of two or more hydrophobic materials are included in the matrix formulations. If an additional hydrophobic material is included, it is preferably a natural or synthetic wax, a fatty acid, a fatty alcohol, or mixtures thereof. Examples include beeswax, carnauba wax, stearic acid and stearyl alcohol.

In other embodiments, the sustained-release matrix comprises digestible, long-chain (e.g., C₈-C₅₀, preferably C₁₂-C₄₀), substituted or unsubstituted hydrocarbons, such as fatty acids, fatty alcohols, glyceryl esters of fatty acids, mineral and vegetable oils and waxes. Hydrocarbons having a melting point of between about 25° C. and about 90° C. are preferred. Of these long-chain hydrocarbon materials, fatty (aliphatic) alcohols are preferred. The oral dosage form can contain up to about 60% (by weight) of at least one digestible, long-chain hydrocarbon.

Further, the sustained-release matrix can contain up to 60% (by weight) of at least one polyalkylene glycol.

In a preferred embodiment, the matrix comprises at least one water-soluble hydroxyalkyl cellulose, at least one C₁₂-C₃₅, preferably C₁₄-C₂₂, aliphatic alcohol and, optionally, at least one polyalkylene glycol. The at least one hydroxyalkyl cellulose is preferably a hydroxy (C₁-C₆) alkyl cellulose, such as hydroxypropylcellulose, hydroxypropylmethylellulose and, preferably, hydroxyethyl cellulose. The amount of the at least one hydroxyalkyl cellulose in die oral dosage form will be determined, amongst other things, by the precise rate of opioid release required. The amount of the at least one aliphatic alcohol in the present oral dosage form will be determined by the precise rate of opioid release required. However, it will also depend on whether the at least one polyalkylene glycol is absent from the oral dosage form.

In certain embodiments, a spheronizing agent, together with the active ingredient, can be spheronized to form spheroids. Microcrystalline cellulose and hydrous lactose impalpable are examples of such agents. Additionally (or alternatively), the spheroids can contain a water-insoluble polymer, preferably an acrylic polymer, an acrylic copolymer, such as a methacrylic acid-ethyl acrylate copolymer, or ethyl cellulose. In such embodiments, the sustained-release coating will generally include a water-insoluble material such as (a) a wax, either alone or in admixture with a fatty alcohol, or (b) shellac or zein.

Preferably, the sequestering subunit comprises the therapeutic agent in sustained-release form. The sustained-release subunit can be prepared by any suitable method. For example a plasticized aqueous dispersion of the release-retarding material can be applied onto the subunit comprising the opioid agonist. A sufficient amount of the aqueous dispersion of release-retarding material to obtain a predetermined sustained-release of the opioid agonist when the coated substrate is exposed to aqueous solutions, e.g., gastric fluid, is preferably applied, taking into account the physical characteristics of the opioid agonist, the manner of incorporation of the plasticizer; etc. Optionally, a further overcoat of a film-former, such as Opadry (Colorcon, West Point, Va.), can be applied after coating with the release-retarding material.

The subunit can be cured in order to obtain a stabilized release rate of the therapeutic agent. In embodiments employing an acrylic coating, a stabilized product can be preferably obtained by subjecting the subunit to oven curing at a temperature above the glass transition temperature of the plasticized acrylic polymer for the required time period. The optimum temperature and time for the particular formulation can be determined by routine experimentation.

Once prepared, the subunit can be combined with at least one additional subunit and, optionally, other excipients or drugs to provide an oral dosage form.

In addition to the above ingredients, a sustained-release matrix also can contain suitable quantities of other materials, e.g., diluents, lubricants, binders, granulating aids, colorants, flavorants and glidants that are conventional in the pharmaceutical art.

Optionally and preferably, the mechanical fragility of any of the sequestering subunits described herein is the same as the mechanical fragility of the therapeutic agent in releasable form. In this regard, tampering with the composition of the invention in a manner to obtain the therapeutic agent will result in the destruction of the sequestering subunit, such that the antagonist is released and mixed in with the therapeutic agent. Consequently, the antagonist cannot be separated from the therapeutic agent, and the therapeutic agent cannot be administered in the absence of the antagonist. Methods of assaying the mechanical fragility of the sequestering subunit and of a therapeutic agent are known in the art.

The composition of the invention can be in any suitable dosage form or fomulation, (see, e.g., Pharmaceutics and Pharmacy Practice, J. B. Lippincott Company, Philadelphia, Pa., Banker and Chalmers, eds., pages 238-250 (1982)). Formulations suitable for oral administration can consist of (a) liquid solutions, such as an effective amount of the inhibitor dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscannellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

One of ordinary skill in the at will readily appreciate that the compositions of the invention can be modified in any number of ways, such that the therapeutic efficacy of the composition is increased through the modification. For instance, the therapeutic agent or sequestering subunit could be conjugated either directly or indirectly through a linker to a targeting moiety. The practice of conjugating therapeutic agents or sequestering subunits to targeting moieties is known in the art. See, for instance, Wadwa et al., J. Drug Targeting 3: 11 (1995), and U.S. Pat. No. 5,087,616. The term “targeting moiety” as used herein, refers to any molecule or agent that specifically recognizes and binds to a cell-surface receptor, such that the targeting moiety directs the delivery of the therapeutic agent or sequestering subunit to a population of cells on which the receptor is expressed. Targeting moieties include, but are not limited to antibodies, or fragments thereof, peptides, hormones, growth factors, cytokines, and any other naturally- or non-naturally-existing ligands, which bind to cell-surface receptors. The term “linker” as used herein, refers to any agent or molecule that bridges the therapeutic agent or sequestering subunit to the targeting moiety. One of ordinary skill in the art recognizes that sites on the therapeutic agent or sequestering subunit, which are not necessary for the function of the agent or sequestering subunit, are ideal sites for attaching a linker and/or a targeting moiety, provided that the linker and/or targeting moiety, once attached to die agent or sequestering subunit, do(es) not interfere with the function of the therapeutic agent or sequestering subunit.

With respect to the present inventive compositions, the composition is preferably an oral dosage form. By “oral dosage form” is meant to include a unit dosage form prescribed or intended for oral administration comprising subunits. Desirably, the composition comprises the sequestering subunit coated with the therapeutic agent in releasable form, thereby forming a composite subunit comprising the sequestering subunit and the therapeutic agent. Accordingly, the invention further provides a capsule suitable for oral administration comprising a plurality of such composite subunits.

Alternatively, the oral dosage form can comprise any of the sequestering subunits of the invention in combination with a therapeutic agent subunit, wherein the therapeutic agent subunit comprises the therapeutic agent in releasable form. In this respect, the invention provides a capsule suitable for oral administration comprising a plurality of sequestering subunits of the invention and a plurality of therapeutic subunits, each of which comprises a therapeutic agent in releasable form.

The invention further provides tablets comprising a sequestering subunit of the invention and a therapeutic agent in releasable form. For instance, the invention provides a tablet suitable for oral administration comprising a first layer comprising any of the sequestering subunits of the invention and a second layer comprising therapeutic agent in releasable form, wherein the first layer is coated with the second layer. The first layer can comprise a plurality of sequestering subunits. Alternatively, the first layer can be or can consist of a single sequestering subunit. The therapeutic agent in releasable form can be in the form of a therapeutic agent subunit and the second layer can comprise a plurality of therapeutic subunits. Alternatively, the second layer can comprise a single substantially homogeneous layer comprising the therapeutic agent in releasable form.

When the blocking agent is a system comprising a first antagonist-impermeable material and a core, the sequestering subunit can be in one of several different forms. For example, the system can further comprise a second antagonist-impermeable material, in which case the sequestering unit comprises an antagonist, a first antagonist-impermeable material, a second antagonist-impermeable material, and a core. In this instance, the core is coated with the first antagonist-impermeable material, which, in turn, is coated with the antagonist, which, in turn, is coated with the second antagonist-impermeable material. The first antagonist-impermeable material and second antagonist-impermeable material substantially prevent release of the antagonist from the sequestering subunit in the gastrointestinal tract for a time period that is greater than 24 hours. In some instances, it is preferable that the first antagonist-impermeable material is the same as the second antagonist-impermeable material. In other instances, the first antagonist-impermeable material is different from the second antagonist-impermeable material. It is within the skill of the ordinary artisan to determine whether or not the first and second antagonist-impermeable materials should be the same or different. Factors that influence the decision as to whether the first and second antagonist-impermeable materials should be the same or different can include whether a layer to be placed over the antagonist-impermeable material requires certain properties to prevent dissolving part or all of the antagonist-impermeable layer when applying the next layer or properties to promote adhesion of a layer to be applied over die antagonist-impermeable layer.

Alternatively, the antagonist can be incorporated into the core, and the core is coated with the first antagonist-impermeable material. In this case, the invention provides a sequestering subunit comprising an antagonist, a core and a first antagonist-impermeable material, wherein the antagonist is incorporated into the core and the core is coated with the first antagonist-impermeable material, and wherein the first antagonist-impermeable material substantially prevents release of the antagonist from the sequestering subunit in the gastrointestinal tract for a time period that is greater than 24 hours. By “incorporate” and words stemming therefrom, as used herein is meant to include any means of incorporation, e.g., homogeneous dispersion of the antagonist throughout the core, a single layer of the antagonist coated on top of a core, or a multi-layer system of the antagonist, which comprises the core.

In another alternative embodiment, the core comprises a water-insoluble material, and the core is coated with the antagonist, which, in turn, is coated with the first antagonist-impermeable material. In this case, the invention further provides a sequestering subunit comprising an antagonist, a first antagonist-impermeable material, and a core, which comprises a water-insoluble material, wherein the core is coated with the antagonist, which, in turn, is coated with the first antagonist-impermeable material, and wherein the first antagonist-impermeable material substantially prevents release of the antagonist from the sequestering subunit in the gastrointestinal tract for a time period that is greater than 24 hours. The term “water-insoluble material” as used herein means any material that is substantially water-insoluble. The term “substantially water-insoluble” does not necessarily refer to complete or 100% water-insolubility. Rather, there are varying degrees of water insolubility of which one of ordinary skill in the art recognizes as having a potential benefit. Preferred water-insoluble materials include, for example, microcrystalline cellulose, a calcium salt, and a wax. Calcium salts include, but are not limited to, a calcium phosphate (e.g., hydroxyapatite, apatite; etc.), calcium carbonate, calcium sulfate, calcium stearate, and the like. Waxes include, for example, carnuba wax, beeswax, petroleum wax, candelilla wax, and the like.

In one embodiment, the sequestering subunit includes an antagonist and a seal coat where the seal coat forms a layer physically separating the antagonist within the sequestering subunit from the agonist which is layered upon the sequestering subunit. In one embodiment, the seal coat comprises one or more of an osmotic pressure regulating agent, a charge-neutralizing additive, a sequestering polymer hydrophobicity-enhancing additive, and a first sequestering polymer (each having been described above). In such embodiments, it is preferred that the osmotic pressure regulating agent, charge-neutralizing additive, and/or sequestering polymer hydrophobicity-enhancing additive, respectively where present, are present in proportion to the first sequestering polymer such that no more than 10% of the antagonist is released from the intact dosage form. Where an opioid antagonist is used in the sequestering subunit and the intact dosage form includes an opioid agonist, it is preferred that ratio of the osmotic pressure regulating agent, charge-neutralizing additive, and/or sequestering polymer hydrophobicity-enhancing additive, respectively where present, in relation to die first sequestering polymer is such that the physiological effect of the opioid agonist is not diminished when the composition is in its intact dosage form or during the normal course digestion in the patient. Release may be determined as described above using the USP paddle method (optionally using a buffer containing a surfactant such as Triton X-100) or measured from plasma after administration to a patient in the fed or non-fed state. In one embodiment, plasma naltrexone levels are determined; in others, plasma 6-beta naltrexol levels are determined. Standard tests may be utilized to ascertain the antagonist's effect on agonist function (i.e., reduction of pain).

The sequestering subunit of the invention can have a blocking agent that is a tether to which the antagonist is attached. The term “tether” as used herein refers to any means by which the antagonist is tethered or attached to the interior of the sequestering subunit, such that the antagonist is not released, unless the sequestering subunit is tampered with. In this instance, a tether-antagonist complex is formed. The complex is coated with a tether-impermeable material, thereby substantially preventing release of the antagonist from the subunit. The term “tether-impermeable material” as used herein refers to any material that substantially prevents or prevents the tether form permeating through the material. The tether preferably is an ion exchange resin bead.

The invention further provides a tablet suitable for oral administration comprising a single layer comprising a therapeutic agent in releasable form and a plurality of any of the sequestering subunits of the invention dispersed throughout the layer of the therapeutic agent in releasable form. The invention also provides a tablet in which the therapeutic agent in releasable form is in the form of a therapeutic agent subunit and the tablet comprises an at least substantially homogeneous mixture of a plurality of sequestering subunits and a plurality of subunits comprising the therapeutic agent.

In preferred embodiments, oral dosage forms are prepared to include an effective amount of melt-extruded subunits in the form of multiparticles within a capsule. For example, a plurality of the melt-extruded multiparticulates can be placed in a gelatin capsule in an amount sufficient to provide an effective release dose when ingested and contacted by gastric fluid.

In another preferred embodiment, the subunits, e.g., in the form of multiparticulates, can be compressed into an oral tablet using conventional tableting equipment using standard techniques. Techniques and compositions for making tablets (compressed and molded), capsules (hard and soft gelatin) and pills are also described in Remington's Pharmaceutical Sciences, (Aurther Osol., editor), 1553-1593 (1980), which is incorporated herein by reference. Excipients in tablet formulation can include, for example, an inert diluent such as lactose, granulating and disintegrating agents, such as cornstarch, binding agents, such as starch, and lubricating agents, such as magnesium stearate.

In yet another preferred embodiment, the subunits are added during the extrusion process and the extrudate can be shaped into tablets as set forth in U.S. Pat. No. 4,957,681 (Klimesch et al.), which is incorporated herein by reference.

Optionally, the sustained-release, melt-extruded, multiparticulate systems or tablets can be coated, or the gelatin capsule can be further coated, with a sustained-release coating, such as the sustained-release coatings described herein. Such coatings are particularly useful when the subunit comprises an opioid agonist in releasable form, but not in sustained-release form. The coatings preferably include a sufficient amount of a hydrophobic material to obtain a weight gain level form about 2 to about 30 percent, although the overcoat can be greater, depending upon the physical properties of the particular opioid analgesic utilized and the desired release rate, among other things.

The melt-extruded dosage forms can further include combinations of melt-extruded multiparticulates containing one or more of the therapeutically active agents before being encapsulated. Furthermore, the dosage forms can also include an amount of an immediate release therapeutic agent for prompt therapeutic effect. The immediate release therapeutic agent can be incorporated or coated on the surface of the subunits after preparation of the dosage forms (e.g., controlled-release coating or matrix-based). The dosage forms can also contain a combination of controlled-release beads and matrix multiparticulates to achieve a desired effect.

The sustained-release formulations preferably slowly release the therapeutic agent, e.g., when ingested and exposed to gastric fluids, and then to intestinal fluids. The sustained-release profile of the melt-extruded formulations can be altered, for example, by varying the amount of retardant, e.g., hydrophobic material, by varying the amount of plasticizer relative to hydrophobic material, by the inclusion of additional ingredients or excipients, by altering the method of manufacture; etc.

In other embodiments, the melt-extruded material is prepared without the inclusion of the subunits, which are added thereafter to the extrudate. Such formulations can have the subunits and other drugs blended together with the extruded matrix material, and then the mixture is tableted in order to provide a slow release of the therapeutic agent or other drugs. Such formulations can be particularly advantageous, for example, when the therapeutically active agent included in the formulation is sensitive to temperatures needed for softening the hydrophobic material and/or the retardant material.

In certain embodiments, the release of the antagonist of the sequestering subunit or composition is expressed in terms of a ratio of the release achieved after tampering, e.g., by crushing or chewing, relative to the amount released from the intact formulation. The ratio is, therefore, expressed as [Crushed]:[Whole], and it is desired that this ratio have a numerical range of at least about 4:1 or greater (e.g., crushed release within 1 hour/intact release in 24 hours). In certain embodiments, the ratio of the therapeutic agent and the antagonist, present in the sequestering subunit, is about 1:1, about 50:1, about 75:1, about 100:1, about 150:1, or about 200:1, for example, by weight, preferably about 1:1 to about 20:1 by weight or 15:1 to about 30:1 by weight. The weight ratio of the therapeutic agent to antagonist refers to the weight of the active ingredients. Thus, for example, the weight of the therapeutic agent excludes the weight of die coating, matrix, or other component that renders the antagonist sequestered, or other possible excipients associated with the antagonist particles. In certain preferred embodiments, the ratio is about 1:1 to about 10:1 by weight. Because in certain embodiments the antagonist is in a sequestered from, the amount of such antagonist within the dosage form can be varied more widely than the therapeutic agent/antagonist combination dosage forms, where both are available for release upon administration, as the formulation does not depend on differential metabolism or hepatic clearance for proper functioning. For safety reasons, the amount of the antagonist present in a substantially non-releasable form is selected as not to be harmful to humans, even if fully released under conditions of tampering.

The compositions of the invention are particularly well-suited for use in preventing abuse of a therapeutic agent. In this regard, the invention also provides a method of preventing abuse of a therapeutic agent by a human being. The method comprises incorporating the therapeutic agent into any of the compositions of the invention. Upon administration of the composition of the invention to the person, the antagonist is substantially prevented from being released in the gastrointestinal tract for a time period that is greater than 24 hours. However, if a person tampers with the compositions, the sequestering subunit, which is mechanically fragile, will break and thereby allow the antagonist to be released. Since the mechanical fragility of the sequestering subunit is the same as the therapeutic agent in releasable form, the antagonist will be mixed with the therapeutic agent, such that separation between the two components is virtually impossible.

The effectiveness of treatment of chronic moderate to severe pain (focusing on osteoarthritis of the hip or knee) is typically measured by mean change in diary Brief Pain Inventory (BPI) score of average pain (daily scores of average pain averaged over 7 days; in-clinic BPI and/or daily diary BPI (worst, least, and current pain)), WOMAC Osteoarthritis Index. Medical Outcomes Study (MOS) Sleep Scale, Beck Depression Inventory, and Patient Global Impression of Change (PGIC). The safety and tolerability of opioid medications such as Kadian NT are compared to placebo using Adverse Events (AEs), clinical laboratory data, vital signs, and two measures of opioid withdrawal: Subjective Opiate Withdrawal Scale (SOWS) and Clinical Opiate Withdrawal Scale (COWS).

The Beck Depression Inventory is a self-administered, 2-item test in multiple-choice format that measures the presence and degree of depression (Beck et al. An inventory for measuring depression. Arch Gen Psych. 1961; 4:561-571). Each of the inventory questions corresponds to a specific category of depressive symptom and/or attitude. Answers are scored on a 0 to 3 scale, where “0” is minimal and “3” is severe. A score of <15 indicates mild depression, a score of 15-30 indicates moderate depression, and a score >30 indicates severe depression.

The WOMAC Osteoarthritis Index consists of questions on three subscales: Pain, Stiffness, and Physical Function (Bellamy et al. Validation study of WOMAC: a health status instrument for measuring clinically important patient relevant outcomes to antirheurnatic drug therapy in patients with osteoarthritis of the hip or knee. J Rheumatol. 1988; 15:1833-1840; Bellamy N. Pain assessment in osteoarthritis: experience with the WOMAC osteoarthritis index. Semin Arthritis Rheum. 1989; 18:14-17; Bellamy et al. Double blind randomized controlled trial of sodium meclofenamate (Meclomen) and diclofenac sodium (Voltaren): post validation reapplication of the WOMAC Osteoarthritis index. J Rheumatol. 192; 19:153-159). Questions are typically completed by the subject before any other efficacy assessments are performed. A typical WOMAC survey is reproduced below:

The PGIC is a self-administered instrument that measures change in patient's overall status on a scale ranging from 1 (very much improved) to 7 (very much worse). The PGIC is based on the Clinical Global Impression of Change (CGIC) (Guy W. ECDEU assessment manual for psychopharmacology. Washington, D.C.: Department of Health, Education and Welfare, 1976; 217-222. Publication Number (ADM) 76-338), which is a validated scale. A typical form of the PGIC survey is shown below:

How would you rate your overall status since your last visit?
(Please circle one)

| | |
|---|---|
| Very Much Improved | 1 |
| Much Improved | 2 |
| Minimally Improved | 3 |
| No Change | 4 |
| Minimally Worse | 5 |
| Much Worse | 6 |
| Very Much Worse | 7 |

Any or all of these measures of effectiveness may be used alone or in combination to determine the efficacy of various formulations or treatment regimens. Provided herein are methods for treating pain in a person comprising administering thereto a multilayer pharmaceutical composition as described herein such that pain is substantially relieved in the patient. By “substantially relieved” is meant that the person reports a decrease in pain as measured by any of several known methods (including but not limited to those described herein) for determining pain. This decrease may be in comparison to no treatment, a placebo, or another form of treatment including but not limited to another composition, either one described herein or otherwise available to one of skill in the art. Typically but not necessarily, pain is considered substantially relieved where the decrease is significant (e.g., p<0.05). The methods described herein provide methods for substantially relieving pain (e.g, providing an analgesic effect) for time periods of at least one week (e.g., two, four, eight, 12, 16, 20, 24, 28, 32, 36, 40 and 100 weeks) by administering a multi-layer pharmaceutical composition as described herein. In one embodiment, the method includes regularly administering (e.g., at least once, twice, three, or four times daily) a multi-layer pharmaceutical composition comprising an agonist and an atagonist as described herein for at least one week (e.g., one, two, four, eight, 12, 16, 20, 24, 28, 32, 36, 40 and 100 weeks) wherein no substantial release (e.g., zero, or less than about 10%, 20%, or 30% release) of the antagonist is observed. In some embodiments, administration of the composition to a population once daily for a time period of at least one week results in no substantial release in at least about 90%, 80%, 70%, 60%, or 50 of the individuals making up the population. Release may be measured by detecting naltrexone or β-naltrexol in plasma.

A better understanding of the present invention and of its many advantages will be had from the following examples, given by way of illustration.

### EXAMPLES

Exemplary KadianNT formulations and methods described below in Examples 1-4 may also be found in PCT/US2007/014282 (WO 2007/149438 A2), PCT/US2007/021627 (WO 2008/063301 A2), and PCT/US08/10357.

### Example 1

Optimization Study #4, KadianNT, Morphine sulfate
and Naltrexone HCl 60 mg/4.8 mg (20-780-1N)

| PI-1495 | | PI-1496 | |
|---|---|---|---|
| mg/unit | Percent | mg/unit | Percent |

Sealed-coated sugar spheres

| | | | | |
|---|---|---|---|---|
| Sugar spheres (#25-30 mesh) | 37.2 | 11.7 | 37.1 | 11.9 |
| Ethylcellulose N50 | 6.2 | 1.9 | 6.2 | 2.0 |
| Mag Stearate | 2.5 | 0.8 | 2.5 | 0.8 |
| DBS | 0.6 | 0.2 | 0.6 | 0.2 |
| Talc | 15.5 | 4.9 | 15.5 | 5.0 |
| Subtotal | 62.0 | 19.4 | 61.9 | 19.9 |

Naltrexone cores

| | | | | |
|---|---|---|---|---|
| Sealed sugar spheres | (62.0) | (19.4) | (61.9) | (19.9) |
| Naltrexone HCl | 4.8 | 1.50 | 4.8 | 1.54 |
| HPC (Klucel LF) | 0.9 | 0.3 | 0.9 | 0.3 |
| Ascorbic acid | 0.5 | 0.2 | 0.5 | 0.2 |
| Talc | 2.27 | 0.7 | 2.24 | 0.7 |
| Subtotal | 70.5 | 22.1 | 70.3 | 22.6 |

Naltrexone pellets

| | | | | |
|---|---|---|---|---|
| Naltrexone cores | (70.5) | (22.1) | (70.3) | (22.6) |
| Eudragit RS PO | 53.3 | 16.7 | 53.3 | 17.1 |
| SLS | 1.8 | 0.6 | 1.8 | 0.6 |
| DBS | 5.36 | 1.7 | 5.36 | 1.7 |
| Talc | 52.1 | 16.3 | 52.1 | 16.8 |
| Subtotal | 183.0 | 57.4 | 182.9 | 58.8 |

Naltrexone-morphine cores

| | | | | |
|---|---|---|---|---|
| Naltrexone pellets | (183.0) | (57.4) | (182.9) | (58.8) |
| Morphine sulfate | 59.9 | 18.8 | 59.7 | 19.2 |
| Sodium chloride | 11.2 | 3.5 | | |
| HPC (Klucel LF) | 7.3 | 2.3 | 4.76 | 1.5 |
| HPMC, 3 cps | | | 7.6 | 2.4 |
| Subtotal | 261.4 | 82.0 | 255.0 | 82.0 |

Naltrexone-morphine pellets

| | | | | |
|---|---|---|---|---|
| Naltrexone-morphine cores | (261.4) | (82.0) | (255.0) | (82.0) |
| Ethylcellulose N50 | 19.81 | 6.2 | 19.31 | 6.2 |
| PEG 6000 | 9.16 | 2.9 | 8.9 | 2.9 |
| Eudragit L100-55 | 4.3 | 1.3 | 4.2 | 1.4 |
| DEP | 4.12 | 1.3 | 4 | 1.3 |
| Talc | 20.13 | 6.3 | 19.62 | 6.3 |
| Total | 319.0 | 100.0 | 311.0 | 100.0 |

A. Method of Preparation—
1. Dissolve Ethylcellulose and dibutyl sebacate into ethanol, then disperse talc and magnesium stearate into the solution.
2. Spray the dispersion from 1 onto sugar spheres in a Wurster to form seal-coated sugar spheres (50 μm seal coat).
3. Dissolve Klucel LF and ascorbic acid into 20:80 mixture of water and ethanol. Disperse naltrexone HCl and talc into the solution.
4. Spray the naltrexone dispersion from 3 onto seal-coated sugar spheres from 2 in a Wurster to form naltrexone cores.
5. Dissolve Eudragit RS, sodium lauryl sulfate and dibutyl debacate into ethanol. Disperse talc into the solution.
6. Spray the dispersion from 5 onto naltrexone cores from 4 in a Wurster to form naltrexone pellets.
7. The Naltrexone pellets are dried at 50° C. for 48 hours.
8. Resulting pellets have a Eudragit RS coat thickness of 150 μm for both PI-1495 PI-1496.
9. (Only for PI-1495) Dissolve sodium chloride and hypromellose into water.
10. Dissolve hypromellose into 10:90 mixture of water and ethanol. Disperse morphine sulfate into the solution.
11. (Only for PI-1495) Spray the solution from 9 followed by the dispersion from 10 onto naltrexone pellets in 7 in a rotor to form naltrexone-morphine cores.
12. (Only for PI-1496) Spray the dispersion from 10 onto naltrexone pellets in 7 in a rotor to form naltrexone-morphine cores.
13. Dissolve ethylcellulose, PEG 6000, Eudragit L100-55 and diethyl phthalate into ethanol. Disperse talc into the solution.
14. Spray the dispersion from 12 onto naltrexone-morphine cores in 11 or 12 to form naltrexone-morphine pellets.
15. The pellets are filled into capsules.

B. In-Vitro Drug Release—
1. Method—USP paddle method at 37° C. and 100 rpm
   1 hour in 0.1N HCl, then 72 hours in 0.05M pH 7.5 phosphate buffer
Results—Percent of NT released at 73 hours for PI-1495=0%
   Percent of NT released at 73 hours for PI-1496=0%
2. Method—USP paddle method at 37° C. and 100 rpm
   72 hrs in 0.2% Triton X-100/0.2% sodium acetate/0.002N HCl, pH 5.5
Results—Percent of NT released at 73 hours for PI-1495-0%
   Percent of NT released at 73 hours for PI-1496=0%

### C. In-Vivo Study

This is a single-dose, open-label, two period study in which two groups of eight subjects received one dose of either PI-1495 or PI-1496. Each subject received an assigned treatment sequence based on a randomization schedule under fasting and non-fasting conditions. Blood samples were drawn prior to dose administration and at 0.5 to 168 hours post-dose. Limits of quantitation are 4.00 pg/mL for naltrexone and 0.250 pg/mL for 6-beta-naltrexol. A summary of the pharmacokinetic results is shown in the following tables.

Naltrexone

| | PI-1495 | | PI-1496 | |
|---|---|---|---|---|
| | Fast | Fed | Fast | Fed |
| Tmax (hr) | 54.00 (N = 2) | 14.34 (N = 3) | 55.20 (N = 5) | 41.60 (N = 5) |
| Cmax (pg/mL) | 8.53 | 6.32 (N = 7) | 24.23 (N = 7) | 45.67 (N = 7) |
| AUCₗₐₛₜ (pg * h/mL) | 100.8 | 75.9 (N = 7) | 500.6 (N = 7) | 1265 (N = 7) |
| AUC∞ (pg * h/mL) | — | — | 2105.3 (N = 2) | 3737 (N = 2) |
| T½ (hr) | — | — | 44.56 (N = 2) | 33.17 (N = 2) |

Relative Bioavailability to an oral solution (Dose-adjusted)

| | | | | |
|---|---|---|---|---|
| Cmax Ratio (Test/Solution) | 0.29% | 0.21% | 0.82% | 1.55% |
| AUCₗₐₛₜ Ratio (Test/Solution) | 1.13% | 0.85% | 5.61% | 14.17% |
| AUC∞ Ratio (Test/Solution) | — | — | 22.0% | 39.1% |

N = 8, unless specified otherwise

6-beta-Naltrexol

| | PI-1495 | | PI-1496 | |
|---|---|---|---|---|
| | Fast | Fed | Fast | Fed |
| Tmax (hr) | 69.00 | 41.44 (N = 7) | 70.51 | 67.63 |
| Cmax (pg/mL) | 116.3 | 151.7 (N = 7) | 303.3 | 656.7 |
| AUCₗₐₛₜ (pg * h/mL) | 5043 | 7332 (N-7) | 14653 | 27503 |
| AUC∞ (pg * h/mL) | 5607 | 8449 (N = 6) | 14930 | 27827 |
| T½ (hr) | 20.97 | 16.69 (N = 7) | 16.29 | 22.59 |

Relative Bioavailability to an oral solution (Dose-adjusted)

| | | | | |
|---|---|---|---|---|
| Cmax Ratio (Test/Solution) | 0.47% | 0.62% | 1.23% | 2.67% |
| AUCₗₐₛₜ Ratio | 2.45% | 3.45% | 7.12% | 13.36% |
| (Test/Solution) | | | | |
| AUC∞ Ratio | 2.64% | 3.97% | 7.02% | 13.08% |
| (Test/Solution) | | | | |

N = 8, unless specified otherwise

Kadian NT pellets with naltrexone pellet coat thickness of 150 μm had comparable naltrexone release as NT pellets with 90 μm coat thickness. This comparable NT release may also be attributed from the presence of 50 μm seal coat on the sugar spheres used in Kadian NT pellets. Significant NT sequestering was observed, both at fasting (>97%) and fed states (>96%). Kadian NT pellets containing sodium chloride immediately above the naltrexone pellet coat (PI-1495) had half the release of naltrexone compared to Kadian NT pellet without sodium chloride (PI-1496), consistent with in vitro results. There is again food effect observed. Lag time was significantly reduced.

### Example 2

Optimization Study #5, KadianNT, Morphine sulfate and
Naltrexone HCl 60 mg/2.4 mg (20-903-AU)

PI-1510

| | Mg/unit | Percent |
|---|---|---|
| Sealed sugar spheres | | |
| Sugar spheres (#25-30 mesh) | 39.9 | 12.2 |
| Ethylcellulose N50 | 6.5 | 2.0 |
| Mag Stearate | 2.6 | 0.8 |
| DBS | 0.7 | 0.2 |
| Talc | 16.7 | 5.1 |
| Subtotal | 66.4 | 20.3 |
| Naltrexone cores | | |
| Sealed sugar spheres | (66.4) | (20.3) |
| Naltrexone HCl | 2.4 | 0.73 |
| HPC (Klucel LF) | 0.5 | 0.1 |
| Ascorbic acid | 0.2 | 0.1 |
| Talc | 1.1 | 0.4 |
| Subtotal | 70.6 | 21.6 |
| Naltrexone pellets | | |
| Naltrexone cores | (70.6) | (21.6) |
| Eudragit RS PO | 53.0 | 16.2 |
| SLS | 1.8 | 0.6 |
| DBS | 5.3 | 1.6 |
| Talc | 53.0 | 16.2 |
| Subtotal | 183.7 | 56.2 |
| Naltrexone-morphine cores | | |
| Naltrexone pellets | (183.7) | (56.2) |
| Morphine sulfate | 60.1 | 18.4 |
| Sodium chloride | 12.5 | 3.8 |
| HPC (Klucel LF) | 6.2 | 1.9 |
| Subtotal | 262.4 | 80.2 |
| Naltrexone-morphine pellets | | |
| Naltrexone-morphine cores | (262.4) | (80.2) |
| Ethylcellulose N50 | 22.9 | 7.0 |
| PEG 6000 | 10.6 | 3.2 |
| Eudragit L100-55 | 5.0 | 1.5 |
| DEP | 4.7 | 1.5 |
| Talc | 21.5 | 6.6 |
| Total | 327.1 | 100.0 |

B. Method of Preparation—
1. Dissolve Ethylcellulose and dibutyl sebacate into ethanol, then disperse talc and magnesium stearate into the solution.
2. Spray the dispersion from 1 onto sugar spheres in a Wurster to form seal-coated sugar spheres (50 μm seal coat).
3. Dissolve Klucel LF and ascorbic acid into 20:80 mixture of water and ethanol. Disperse naltrexone HC and talc into the solution.
4. Spray the naltrexone dispersion from 3 onto seal-coated sugar spheres from 2 in a Wurster to form naltrexone cores.
5. Dissolve Eudragit RS, sodium lauryl sulfate and dibutyl sebacate into ethanol. Disperse talc into the solution.
6. Spray the dispersion from 5 onto naltrexone cores from 4 in a Wurster to form naltrexone pellets.
7. The Naltrexone pellets are dried at 50° C. for 48 hours.
8. Resulting pellets have a Eudragit RS coat thickness of 150 μm.
9. Dissolve sodium chloride and hypromellose into water.
10. Dissolve hypromellose into 10:90 mixture of water and ethanol. Disperse morphine sulfate into the solution.
11. Spray the solution from 9 followed by the dispersion from 10 onto naltrexone pellets in 7 in a rotor to form naltrexone-morphine cores.
12. Dissolve ethylcellulose, PEG 6000, Eudragit L100-55 and diethyl phthalate into ethanol. Disperse talc into the solution.
13. Spray the dispersion from 12 onto naltrexone-morphine cores in 11 or 12 to form naltrexone-morphine pellets
14. The pellets are filled into capsules.

B. In-Vitro Drug Release—
1. Method—USP paddle method at 37° C. and 100 rpm
   1 hour in 0.1N HCl, then 72 hours in 0.05M pH 7.5 phosphate buffer
Results—Percent of NT released at 73 hours for =0%
2. Method—USP paddle method at 37° C. and 100 rpm
   72 hrs in 0.2% Triton X-100/0.2% sodium acetate/0.002N HCl, pH 5.5
   Results—Percent of NT released at 73 hours=0%

### C. In-Vivo Study

This is a single-dose, open-label, two period study in which eight subjects were randomized to receive one dose of PI-1510 under either fasted or fed state during Study Period 1 and alternate fasted or fed state for Study Period 2. Blood samples were drawn prior to dose administration and at 0.5 to 168 hours post-dose. Limits of quantitation are 4.00 pg/mL for naltrexone and 0.250 pg/mL for 6-beta-naltrexol. A summary of the pharmacokinetic measurements is provided in the following tables.

6-beta-Naltrexol levels

PI-1510

| | Fast | Fed |
|---|---|---|
| Tmax (hr) | 45.00 (N = 6) | 57.29 (N = 7) |
| Cmax (pg/mL) | 16.1 | 25.0 |
| AUCₗₐₛₜ (pg * h/mL) | 609.2 | 1057 |
| AUC∞ (pg * h/mL) | 1233 | 1431 (N = 6) |
| T½ (hr) | 17.36 | 17.48 (N = 6) |

Relative Bioavailability to an oral solution
(Dose-adjusted)

| | | |
|---|---|---|
| Cmax Ratio (Test/Solution) | 0.44% | 0.68% |
| AUCₗₐₛₜ Ratio (Test/Solution) | 1.97% | 3.42% |
| AUC∞ Ratio (Test/Solution) | 3.86% | 4.49% |

N = 8, unless specified otherwise

It was concluded that PI-1510 and PI-1495 are comparable. The reduction in naltrexone loading in the pellets (from 1.5% in PI-1495 to 0.7% in PI-1510) does not seem to affect NT release. Significant NT sequestering was observed, both at fasting (>96%) and fed states (>95%). The food effect observed was modest in terms of total NT release. However, the lag time was significantly reduced in the presence of food. There were subjects with multiple peaks of release.

### Summary of NT Release from all In-Vivo Studies

| | | BA (AUC last) | BA (AUC inf) |
|---|---|---|---|
| | BA (Cmax) (%) | (%) | (%) |
| OPTIM. #4 | | | |
| PI-1495 | | | |
| Fast | | | |
| Avg ± SD | 0.5 ± 0.5 | 2.5 ± 2.3 | 2.6 ± 2.4 |
| Range | 0.1-1.4 | 5.9-0.3 | 0.3-5.7 |
| Fed | | | |
| Avg ± SD | 3.0 ± 6.7 | 10.2 ± 19.4 | 11.3 ± 20.0 |
| Range | 0.1-19.4 | 0.2-57.0 | 0.2-55.4 |
| Fed (-Subject 1) | | | |
| Avg ± SD | 0.6 ± 0.9 | 3.6 ± 4.9 | 4.0 ± 5.0 |
| Range | 0.1-2.5 | 0.2-13.8 | 0.2-13.4 |
| PI-1496 | | | |
| Fast | | | |
| Avg ± SD | 1.2 ± 0.9 | 7.1 ± 4.6 | 7.0 ± 4.6 |
| Range | 0.1-2.7 | 0.6-14.2 | 0.6-14.5 |
| Fed | | | |
| Avg ± SD | 2.7 ± 2.9 | 13.4 ± 12.6 | 13.1 ± 12.3 |
| Range | 0.1-7.6 | 0.1-31.6 | 0.4-30.7 |
| OPTIM. #5 | | | |
| PI-1510 | | | |
| Fast | | | |
| Avg | 0.4 | 2.0 | 3.9 |
| Fed | | | |
| Avg | 0.7 | 3.4 | 4.5 |

BA (Cmax) = Relative bioavailability based on Cmax = Dose-adjusted ratio of Cmax (NT/KNT pellet) to Cmax (NT soln)
BA (AUC last) = Relative bioavailability based on AUC last = Dose-adjusted ratio of AUC last (NT/KNT pellet) to AU
BA (AUC inf) = Relative bioavailability based on AUC inf = Dose-adjusted ratio of AUC last (NT/KNT pellet)
Total in-vivo cumulative NT release can be extrapolated from BA (AUC inf) calculations from 6-beta-Naltrexol plasma levels

### Example 3

Kadian NT Formulation #6 (AL-01)

| | | Final |
|---|---|---|
| | 15% | formulation |
| | TPCW | AL-01 |
| Seal-coated Sugar Spheres | | |
| Sugar Spheres (#25-30 mesh) | 11.99 | 11.94 |
| Ethylcellulose NF 50 cps | 2.00 | 1.99 |
| Magnesium Stearate NF | 0.80 | 0.80 |
| Dibutyl Sebacate NF | 0.20 | 0.20 |
| Talc USP (Suzorite 1656) | 5.00 | 4.98 |
| Naltrexone HCl Core | | |
| Seal-coated Sugar Spheres | | (19.90) |
| Naltrexone Hydrochloride USP | 0.73 | 0.72 |
| Hydroxypropyl Cellulose NF | 0.14 | 0.14 |
| Ascorbic Acid USP | 0.07 | 0.07 |
| Talc USP (Suzorite 1656) | 0.34 | 0.34 |
| Naltrexone HCl Intermediate Pellet | | |
| Naltrexone HCl Core | | (21.17) |
| Ammonio Methacrylate Copolymer Type B NF | 6.26 | 6.23 |
| Sodium Lauryl Sulfate NF | 0.22 | 0.22 |
| Dibutyl Sebacate NF | 0.63 | 0.62 |
| Talc USP (Suzorite 1656) | 6.08 | 6.05 |
| Naltrexone HCl Finished Pellet | | |
| Naltrexone HCl Intermediate Pellet | | (34.29) |
| Ammonio Methacrylate Copolymer Type B NF | 9.89 | 9.85 |
| Sodium Lauryl Sulfate NF | 0.34 | 0.34 |
| Dibutyl Sebacate NF | 0.99 | 0.98 |
| Talc USP (Suzorite 1656) | 9.71 | 9.67 |
| NaCl Overcoated Naltrexone HCl Pellet | | |
| Naltrexone HCl Finished Pellet | | (55.13) |
| Sodium Chloride USP | 3.75 | 3.73 |
| Hydroxypropyl Cellulose NF | 0.42 | 0.41 |
| MS Cores with Sequestered Naltrexone HCl | | |
| NaCl Overcoated Naltrexone HCl Pellet | | (59.28) |
| Morphine Sulfate USP | 18.11 | 18.03 |
| Hydroxypropyl Cellulose NF | 1.42 | 1.42 |
| MS Extended-release with Sequestered | | |
| Naltrexone HCl Pellet | | |
| MS Cores with Sequestered Naltrexone HCl | | (78.73) |
| Component (a): ethylcellulose NF (50 cps) | 7.40 | 7.36 |
| Component (c): polyethylene glycol NF (6000) | 3.42 | 3.40 |
| Component (b): methacrylic acid copolymer NF | 1.60 | 1.60 |
| (Type C, Powder) | | |
| Diethyl Phthalate NF (plasticizer) | 1.53 | 1.53 |
| Talc USP (Suzorite 1656) (filler) | 6.98 | 7.38 |
| Total | 100.0 | 100.0 |

In certain embodiments, components (a), (b) and/or (c) may be included as described below:
(a) preferably a matrix polymer insoluble at pH of about 1 to about 7.5; preferably ethylcellulose; preferably at least 35% by weight of a+b+c;
(b) preferably an enteric polymer insoluble at pH of about 1 to about 4 but soluble at pH of about 6 to about 7.5; preferably methacrylic acid-ethyl acrylate copolymer (methacrylic acid copolymer type C) preferably about 1 to about 30% of a+b+c; and,
(c) compound soluble at a pH from about 1 to about 4; preferably polyethylene glycol with a molecular weight from about 1700 to about 20.000; preferably from about 1% to about 60% by weight of a+b+c.

C. Method of Preparation
1. Ethylcellulose and Dibutyl Sebacate were dissolved into Alcohol SDA3A. Talc and Magnesium Stearate were then dispersed into the solution. The percent solid of the dispersion was 20%.
2. The dispersion from 1 was sprayed onto Sugar Spheres in a Wurster to form Seal-coated Sugar Spheres (approx. 50 μm seal coat).
3. Hydroxypropyl Cellulose and Ascorbic Acid were dissolved into a 20:80 mixture of Water and Alcohol SDA3A. Naltrexone HCl and Talc were then dispersed into the solution. The percent solid of the dispersion is 20.4%.
4. The Naltrexone HCl dispersion from 3 was sprayed onto Seal-coated Sugar Spheres from 2 in a Wurster to form Naltrexone HCl cores.
5. Ammonio Methacrylate Copolymner. Sodium Lauryl Sulfate and Dibutyl Sebacate were dissolved into a 22:78 mixture of Water and Alcohol SDA3A. Talc was dispersed into the solution. The percent solid of the dispersion was 20%.
6. The dispersion from 5 was splayed onto Naltrexone HCl cores from 4 in a Wurster to form Naltrexone HCl intermediate Pellets.
7. The Naltrexone HCI Intermediate Pellets were dried in an oven at 50° C. for 24 hours.
8. Ammonio Methacrylate Copolymer, Sodium Lauryl Sulfate and Dibutyl Sebacate were dissolved into a 22:78 mixture of Water and Alcohol SDA3A. Talc was dispersed into the solution. The percent solid of the dispersion was 20%.
9. The dispersion from 8 was sprayed onto Naltrexone HCl Intermediate Pellets from 7 in a Wurster to form Naltrexone HCl Finished Pellets.
10. The Naltrexone HCl Finished Pellets were dried in an oven at 50° C. for 24 hours
11. The resulting pellets had a pellet coat thickness of approximately 150 μm.
12. Sodium Chloride (NaCl) and Hydroxypropyl Cellulose were dissolved into Water. The percent solid in the solution was 6%.
13. The Sodium Chloride solution from 12 was sprayed onto Naltrexone HCl Finished Pellets from 10 in a Wurster to form Sodium Chloride (NaCl) Overcoated Naltrexone HCl Pellets.
14. Hydroxypropyl Cellulose was dissolved into Alcohol SDA3A, and Morphine Sulfate dispersed into the solution. The percent solid in the dispersion was 24.4%.
15. The Morphine Sulfate dispersion from 14 was sprayed onto NaCl Overcoated Naltrexone HCl Pellets in 13 in a rotor to form Morphine Sulfate Cores with Sequestered Naltrexone HCl.
16. Ethylcellulose, Polyethylene Glycol, Methacrylic Acid Copolymer and Diethyl Phthalate were dissolved into Alcohol SDA3A. Talc was dispersed into the solution. The percent solid in the dispersion was 14.3%.
17. The Dispersion from 16 was sprayed onto Morphine Sulfate Cores with Sequestered Nultrexone HCl in 15 to form Morphine Sulfate Extended-release with Sequestered Naltrexone HCl Pellets.
18. The pellets were filled into capsules.

### Example 4

### Method for Treating Pain (202)

As an example, Kadian NT (60 mg morphine sulfate, 2.4 mg naltrexone HCl) was administered to humans and compared to the previously described product Kadian. Each Kadian sustained release capsule contains either 20, 30, 50, 60, or 100 mg of Morphine Sulfate USP and the following inactive ingredients common to all strengths: hydroxypropyl methylcellulose, ethylcellulose, methacrylic acid copolymer, polyethylene glycol, diethyl phthalate, talc, corn starch, and sucrose. In these studies, the effects of Kadian were compared to those of Kadian NT.

Patients already being treated with Kadian were subjected to a “washout” period of approximately 14 days during which Kadian was not administered. Immediately following this washout period, the trial was begun. Patients were either administered Kadian or Kadian NT at day 0. After a period of up to 28 days treatment with Kadian®, patients were then “crossed-over” to Kadian NT or continued taking Kadian®. The amount of Kadian NT was individually adjusted such that each patient was receiving approximately the same amount of morphine they had previously been receiving while taking Kadian. This cross-over was then repeated after 14 days. Various physiological responses were measured at different timepoints, as discussed below. These responses included morphine blood levels, naluexone blood levels, 6-β-naltrexol blood levels and pain scores.

Mean morphine concentrations were measured and determined to be approximately the same for Kadian® and Kadian NT. This observation confirms that the new formulation effectively releases morphine into the blood of patients. This is shown in the table below:

| | | | | | |
|---|---|---|---|---|---|
| Cmax | Cmin | Cavg | Tmax | Fluctuation | AUC(TAU) |
| (pg/mL) | (pg/mL) | (pg/mL) | (hr) | (%) | (hr * pg/mL) |

Kadian

| | | | | | | |
|---|---|---|---|---|---|---|
| N | 68 | 68 | 68 | 68 | 68 | 68 |
| Mean | 12,443 | 6,650 | 9,317 | 4.90 | 66.3 | 111,806 |
| SD | 7,680 | 4,544 | 6,019 | 3.36 | 28.8 | 77,223 |
| Min | 2,630 | 1,000 | 1,758 | 0.00 | 21.4 | 21,100 |
| Median | 9,870 | 5,285 | 7,426 | 5.00 | 63.5 | 89,110 |
| Max. | 35,600 | 21,600 | 28,908 | 12.0 | 213 | 346,900 |
| CV % | 61.7 | 68.3 | 64.6 | 68.5 | 43.4 | 64.6 |

Kadian NT

| | | | | | | |
|---|---|---|---|---|---|---|
| N | 68 | 68 | 68 | 68 | 68 | 68 |
| Mean | 13,997 | 6,869 | 10,120 | 4.29 | 71.49 | 121,438 |
| SD | 10,949 | 5,377 | 7,316 | 3.05 | 38.59 | 87,794 |
| Min | 2,420 | 0.00 | 1,815 | 0.00 | 21.04 | 21,775 |
| Median | 10,200 | 5,805 | 7,496 | 4.00 | 65.89 | 89,948 |
| Max. | 57,600 | 29,000 | 35,046 | 12.0 | 265 | 420,550 |
| CV % | 78.2 | 78.3 | 72.3 | 71.0 | 54.0 | 72.3 |

It is important that the Kadian NT formulation not release significant amounts of antagonist (i.e., naltrexone or derivatives thereof) into the bloodstream such that the activity of morphine is diminished. Only 14 of 69 patients had quantifiable (>4.0 pg/mL) naltrexone concentrations. The range of quantifiable concentrations was 4.4-25.5 pg/mL. However, the release of some naltrexone into the bloodstream did not significantly affect the pain scores (see below).

| | Naltrexone | |
|---|---|---|
| | Conc | |
| Subject | (pg/mL) | Pain Score* |
| 49411 | 25.5 | 2 |
| 49408 | 16.8 | 3 |
| 59510 | 15.9 | 2 |
| 29218 | 13.5 | 0 |
| 39308 | 7.74 | 0 |
| 39306 | 8.98 | 1 |
| 49422 | 8.12 | 4 |
| 79709 | 7.15 | 2 |
| 89817 | 6.82 | 3 |
| 59509 | 6.29 | 2 |
| 49409 | 6.58 | 2 |
| 49431 | 4.81 | 1 |
| 49430 | 4.58 | 1 |
| 59530 | 4.4 | 3 |

*A pain score of 0-3 is considered “mild” and 4-7 is considered “moderate”.

When provided in an immediate formulation, naltrexone (parent) is rapidly absorbed and converted to the 6-β-naltrexol metabolite. 6-β-naltrexol is a weaker opioid antagonist than naltrexone, having only 2 to 4% the antagonist potency. Most patients had quantifiable, levels (>0.25 pg/mL) of 6-β-naltrexol. The incidental presence of 6-β-naltrexol in the plasma had no effect on pain scores.

It was also important to confirm that Kadian NT did not result in a significantly different type, number or severity of common adverse events. This was confirmed, as shown below:

| | |
|---|---|
| Open-label | Double-blind |

| | Kadian | Kadian | Kadian NT |
|---|---|---|---|
| Event | (N = 111) | (N = 71) | (N = 71) |
| Any event | 83.8% | 45.1% | 46.5% |
| Constipation | 46.8% | 12.7% | 15.5% |
| Nausea | 40.5% | 8.5% | 9.9% |
| Somnolence | 28.8% | 8.5% | 9.9% |
| Vomiting | 24.3% | 4.2% | 8.5% |
| Dizziness | 20.7% | 7.0% | 1.4% |
| Headache | 16.2% | 8.5% | 4.2% |

In addition, it was important to note whether Kadian NT functioned similarly to Kadian with respect to adverse events typically associated with withdrawal symptoms. This was confirmed as shown below:

| | |
|---|---|
| Open-label | Double-blind |

| | Kadian | Kadian | Kadian NT |
|---|---|---|---|
| Event | (N = 111) | (N = 71) | (N = 71) |
| Tremor | 3.6% | 0.0% | 0.0% |
| Anxiety | 2.7% | 2.8% | 1.4% |
| Irritability | 1.8% | 0.0% | 0.0% |
| Restlessness | 0.9% | 0.0% | 0.0% |
| Muscle Twitch | 0.9% | 0.0% | 0.0% |
| Cold Sweat | 0.9% | 0.0% | 1.4% |
| Piloerection | 0.0% | 0.0% | 0.0% |
| Rhinitis | 0.0% | 0.0% | 0.0% |
| Tachycardia | 0.0% | 0.0% | 0.0% |

Other measurements, including In-Clinic Pain, WOMAC Pain, WOMAC Stiffness, WOMAC Daily Activities, and BPI Pain were also made. It was determined that the differences in these measurements in those taking Kadian and those taking Kadian NT was not significant, as shown below.

In-Clinic Pain (ITT Population, Completers)

| | Mean | | Treatment | 95% CI for |
|---|---|---|---|---|
| Day | Kadian | Kadian NT | P-value | Difference |
| Baseline | 2.13 | | | |
| Change Day 7 | N = 68 | N = 69 | 0.9773 | −0.32, 0.33 |
| | +0.18 | +0.16 | | |
| Change Day 14 | N = 69 | N = 69 | 0.2176 | −0.13, 0.56 |
| | +0.28 | +0.06 | | |

WOMAC Pain (ITT Population, Completers)

| | Mean | | Treatment | 95% CI for |
|---|---|---|---|---|
| Day | Kadian | Kadian NT | P-value | Difference |
| Baseline | 98.1 | | | |
| Change Day | N = 69 | N = 69 | 0.0928 | −2.0, 26.0 |
| 14 | +18.1 | +5.9 | | |

WOMAC Stiffness (ITT Population, Completers)

| | Mean | | Treatment | 95% CI for |
|---|---|---|---|---|
| Day | Kadian | Kadian NT | P-value | Difference |
| Baseline | 51.1 | | | |
| Change Day | N = 69 | N = 69 | 0.0200 | 1.7, 18.5 |
| 14 | +12.3 | +2.1 | | |

WOMAC Daily Activities (ITT Population, Completers)

| | Mean | | Treatment | 95% CI for |
|---|---|---|---|---|
| Day | Kadian | Kadian NT | P-value | Difference |
| Baseline | 396.6 | | | |
| Change Day | N = 69 | N = 69 | 0.1206 | −11.0, 93.6 |
| 14 | +70.7 | +28.9 | | |

In conclusion, plasma morphine levels for Kadian and Kadian NT are bioequivalent. It was observed that 55 of 69 (80%) patients had no measurable levels of naltrexone. Of the 14 patients with measurable levels of naltrexone, there was no negative effect on pain scores. Seven of these 14 patients had a measurable level at only one time point. Most patients had some level of 6-β-naltrexol, however there was no negative effect on pain scores. In addition, there was no difference in pain scores in individuals taking Kadian or Kadian NT.

### Example 5

### Long-Term Kadian NT Efficacy Study (301)

### A. Study Design

This study was a randomized, double-blind, and placebo-controlled study in subjects with moderate to severe chronic pain due to osteoarthritis (OA) of the hip or knee. The primary objective of this study was to evaluate the efficacy of Kadian NT (twice daily (BID)) compared with placebo for the treatment of chronic moderate to severe pain (focusing on osteoarthritis of the hip or knee) as measured by mean change in diary BPI score of average pain (daily scores of average pain averaged over 7 days) from randomization to 12 weeks following randomization. The secondary objectives were: 1) to evaluate the efficacy of Kadian NT (BID) compared with placebo as measured by in-clinic BPI, daily diary BPI (worst, least, and current pain), WOMAC Osteoarthritis Index, Medical Outcomes Study (MOS) Sleep Scale. Beck Depression inventory, and Patient Clobal Impression of Change (PGIC); and, 2) To evaluate the safety and tolerability of Kadian NT compared to placebo using AEs, clinical laboratory data, vital signs, and two measures of opioid withdrawal: Subjective Opiate Withdrawal Scale (SOWS) and Clinical Opiate Withdrawal Scale (COWS).

For this study, the Baseline Visit (Day 0) was labeled Visit X. Subsequent visits in the Titration Phase of the study were labeled Visit X+1 Week, Visit X+2 Weeks, etc. The first visit in the Maintenance Phase is labeled Visit Y, and subsequent visits in this phase of the study are Visit Y+1 Week, Visit Y+2 Weeks. Visit Y+4 Weeks, etc.

### 1. Screening Visit

Potential subjects were screened up to 14 days (Days −14 to −1) prior to a Baseline Visit (Visit X/Day 0). At the Screening Visit, the informed consent was reviewed and signed; inclusion and exclusion criteria and medical history was reviewed; standard clinical laboratory tests, hepatitis serology tests, and a 12-lead electrocardiogram (ECG) will be performed, a complete physical examination (including height, weight, and Body Mass Index (BMI) calculation) were be performed; and, vital signs (blood pressure, heat rate, respiratory rate, and body temperature) were recorded. In addition, a urine drug screen was performed for all subjects. A urine pregnancy test was performed for all female subjects of childbearing potential.

### 2. Washout Period

During the Washout Period (a 1 to 7 day period during the Day −14 to Day −1 Screening), subjects were instructed to stop taking all prohibited medications and pain medications. Subjects used an electronic diary to answer daily questions about deir pain score and use of rescue medication. Once the required pain score has been achieved (defined as an average 24-hour pain intensity of ≧5 on the 11-point Brief Pain Inventory (BPI) scale), the subject was instructed by die electronic diary to contact the site and return to the clinic for the Baseline Visit within 72 hours of having achieved the pain score of ≧5. If the subject still had not achieved a pain intensity of ≧5 on the 11-point BPI scale by the end of the Washout Period, then the subject was discontinued.

### 3. Baseline Visit

Subjects returned for their Baseline Visit to revisit inclusion and exclusion criteria, perform standard clinical laboratory tests (including urine drug screen and urine pregnancy test), record vital signs, assess adverse events (AEs) and concomitant medications, complete the Medical Outcomes Study (MOS) Sleep Scale, the Beck Depression Inventory, and the Western Ontario and McMaster Universities (WOMAC) Osteoarthritis Index, and assess pain level using the Brief Pain Inventory (BPI). Subjects who meet all inclusion/exclusion criteria and who have an average 24-hour pain intensity of ≧5 on the 11-point BPI scale completed the Baseline Visit, enter the Titration Phase and begin titrating to an effective dose of open-label Kadian NT. If subjects did not meet the 24-hour pain intensity inclusion criteria (i.e., 24-hour pain intensity score is not ≧5) at the Baseline Visit, they were not allowed to re-qualify for entry into the Titration Phase of the study. Subjects who are unable to tolerate their pain with the maximum allowed rescue medication were be discontinued from the study.

### 4. Titration Phase

During the Titration Phase, all opioid naïve subjects (defined as a subject who has not received any opioid in the last 30 days) started with 20 mg Kadian NT at bedtime for the first 3 nights. If the subject was taking opioids prior to the washout, the starting dose was 20 mg BID (with the first dose taken at bedtime). The dose of study drug was titrated up or down to find a Kadian NT BID dose that managed the subject's pain. Dosage titrations (up or down) were made weekly, however, if needed, and subjects were titrated up after being on their current dose for at least 3 days (72 hours). Increases in Kadian NT dosing during the titration period proceeded by total daily dose increases of 20 mg (with the exception of a 40 mg daily dose increase if titrating from 120 mg/day to 160 mg/day). The maximum allowed dose was 80 mg BID (160 mg/day). Two back-titrations (dose reductions) were allowed if necessary to establish the tolerated effective dose. All patients were given a daily prophylactic bowel regimen for constipation. Subjects were dispensed an electronic rake-home diary for daily pain assessments and rescue medication (acetaminophen up to 500 mg every 6 hours as needed). Subjects returned for weekly visits during the titration period. At each visit during titration, vital signs were recorded, and diaries collected, reviewed and re-dispensed, and study medication will be returned and dispensed as appropriate. Adverse events (AE), and concomitant medications including rescue medication were assessed and recorded. Pain levels were also assessed using the BPI at each visit. The maximum duration of the Titration Phase was 45 days.

A subject was considered a treatment responder (reached an “effective dose”) when the average score of the “pain on the average in die last 24 hours” (question #3) is ≦4 on the 11-point BI scale over the last 4 day period prior to the clinic visit as collected in the diary width a minimum 2 point decrease from baseline. All treatment responders were randomized into the study. If this criterion was not met by the end of the Titration Phase, or if a subject is not able to complete the titration due to lack of efficacy, AE and/or other reason(s), or if a subject's pain is not managed with ≧20 and ≦80 mg BID of Kadian NT, an Early Termination Visit was completed.

Once a subject was identified as a treatment responder, they continued dose titration for increased pain relief prior to being randomized. However, the subjects did not receive a dose greater than 160 mg/day nor titrated longer than the maximum allowable number of 45 days.

### 5. Maintenance Phase

Subjects who successfully completed the Titration Phase entered the Maintenance Phase (Visit Y) and were randomized to receive either the same effective dose of Kadian NT achieved in die Titration Phase or placebo. Subjects randomized to the placebo arm were force tapered gradually from Kadian NT to placebo (in a blinded fashion using a double-dummy design) and all subjects, whether receiving Kadian NT or placebo, were assessed for signs of withdrawal during the tapering period of the Maintenance Phase. In the Maintenance Phase, subjects had visits on days 0 (Visit Y), Visit Y+1 week, and Visit Y+2 weeks and then visits every 2 weeks up to 12 weeks (Visits Y+4, 6, 8, 10, and 12 Weeks). At each visit, vital signs were recorded, diaries collected, reviewed and re-dispensed as appropriate, study medication and rescue medication returned and dispensed as appropriate. AEs, concomitant medications, and rescue medications were assessed and recorded, the MOS Sleep Scale (Visit Y, and Visits Y+4, 8, and 12 Weeks), the WOMAC Osteoarthritis Index and the Patient Global Impression of Change (PGIC) (except at Visit Y+1 Week), and the Beck Depression Inventory (Visits Y+4, 8, and 12 Weeks) were completed, and pain level assessed. The Clinical Opiate Withdrawal Scale (COWS) was performed at Day 0 (Visit Y), Visits Y+1 Week, Y+2 Weeks, Y+12 Weeks and at the Early Termination Visit (if applicable). The Subjective Opiate Withdrawal Scale (SOWS) was completed daily for the first 2 weeks of the Maintenance Phase. In addition, at Visit Y+12 Weeks, a physical examination (including weight) and standard clinical laboratory tests was performed. The electronic diary was not be dispensed at Visit Y+12 Weeks. Subjects completing the Maintenance Phase will completed a two-week tapering period and were scheduled for a Post-Treatment Follow-Up visit at the end of the taper to record vital signs, assess and record AEs and concomitant medications, aid arrange appropriate transition to standard of care for the existing OA condition.

### 6. Early Termination

Subjects who prematurely withdrew from the Titration Phase of the study completed an Early Termination Visit that included COWS and the same procedures as the final visit in the Maintenance Phase (Visit Y+12 Weeks) except for the MOS Sleep Scale, the Beck Depression Inventory, and the WOMAC Osteoarthritis Index. These subjects were asked to return for a Post-Treatment Follow-Up visit as described previously. Subjects who prematurely terminated from the Titration Phase were not provided a blister card for the two-week taper period. Instead, the investigator was free to choose to taper subjects via IWRS by gradually selecting lower dosage strengths. For this study, rescue medication was allowed in the form of sponsor provided acetaminophen (500 mg every 6 hours as needed) during the Washout. Titration, and Maintenance Phases

### 7. Study Medications

Study medications were in the form of capsules administered orally. Study medications are: 1) Kadian NT 20, 30, 40, 50, 60, and 80 mg capsules; 2) placebo to match the above Kadian NT capsules; or, 3) acetaminophen (up to 500 mg every 6 hours as needed) as rescue medication.

### 8. Study Population

It was assumed that 5% of the subjects who enter the open-label Titration Phase would be randomized into the double-blind Maintenance Phase, and that approximately 728 subjects recruited to enter the Titration Phase to achieve approximately 200 subjects in each of the two treatment groups (Kadian NT or placebo) in the Maintenance Phase. The inclusion criteria are shown below:
1) Subject is 21 years of age or older and exhibits sufficient literary skills to complete study assessments.
2) If female, subject is either nor of childbearing potential (defined as postmenopausal for at least one year or surgically sterile [bilateral tubal ligation, bilateral oophorectomy or hysterectomy]) or subject is of childbearing potential and practicing one of the following methods of birth control:
   total abstinence front sexual intercourse (minimum one complete menstrual cycle before study entry);
   a vasectomized partner;
   contraceptives (oral, parenteral, or transdermal) for three consecutive months prior to investigational product administration;
   intrauterine device (IUD); or,
   double-barrier method (condoms, sponge, diaphragm or vaginal ring with jellies or cream).
3) If female of childbearing potential, subject has a negative urine pregnancy test at screening (urine specimen must be obtained within 14 days prior to Baseline);
4) Subject is judged to be in generally good health at screening based upon the results of a medical history, physical examination, laboratory profile, and 12-lead ECG;
5) Subject is able to communicate meaningfully and comply with all study procedures;
6) Subject must voluntarily sign and date an informed consent form, approved by an Institutional Review Board (IRB)/Independent Ethics Committee (IEC), prior to the conduct of any study-specific procedures;
7) Subject required treatment of target joint pain within the last 90 days and meets at least one of the following criteria:
   is unable to consistently control target joint pain with non-opioid analgesics (e.g. therapeutic doses of nonsteroidal anti-inflammatory drugs [NSAIDs], cyclooxygenase-II [COX-II] inhibitors), or tramadol OR
   currently requires opioid treatment (single or combination product) for target joint pain, with the equivalent of ≦40 mg/day of oral morphine sulfate, inclusive of breakthrough pain medication.
8) Subject has an average 24-hour pain intensity of ≧5 on the 11-point BPI scale at the Baseline Visit;
9) Subject has a primary diagnosis of Functional Class I-III OA of the hip or knee and subject meets American College of Rheumatology (ACR) clinical classification criteria for osteoarthritis of the hip and knee, defined by the following:
   In the case knee OA: knee pain and at least 3 of age >50; morning stiffness <30 minutes; crepitus on active motion; bony tenderness; bony enlargement; and, no palpable warmth of synovium;
   In the case of hip OA; hip pain and; decreased range of movement (ROM) (internal rotation of the hip ≦15° AND hip flexion ≦115°) OR age >50; morning Stiffness ≦60 minutes, and, pain with hip internal rotation;
   If more than one potential joint met die above listed criteria at baseline, the subject was directed to choose the most painful joint to serve as the target joint for this study. The target joint was not allowed to contain any type of orthopedic and/or prosthetic device.

A subject was excluded from the study if he/she met any of the following criteria:
1) Documented history of an allergic reaction (hives, rash, etc.) or a clinically significant intolerance to morphine or other opioids, such that treatment with morphine is contraindicated;
2) Pregnant and/or breast-feeding;
3) Clinically significant infection injury/illness within one month prior to screening;
4) Receiving systemic chemotherapy or had an active malignancy of any type, or had been diagnosed with cancer within the past three years (excluding squamous or basal cell carcinoma of the skin);
5) Documented history of drug abuse/dependence/misuse or narcotic analgesic abuse/dependence/misuse within five years prior to screening;
6) History of alcohol abuse/dependence within five years prior to screening, which, in the opinion of the investigator, may have influenced subject compliance with the study;
7) Positive result for non-prescription drugs of abuse at screening (e.g. cocaine, hernia, marijuana);
8) Sitting systolic blood pressure >180 mmHg or <90 mmHg, and/or a sitting diastolic blood pressure >120 mmHg or <0.0 mmHg at screening;
9) BMI>45 kg/m²;
10) Beck Depression Index score ≦18 at Baseline or has an established history of major depressive disorder that is not controlled with medication;
11) Introduction of physiotherapy without four-week stabilization period (except transcutaneous electrical nerve stimulation (TENS) which is not allowed);
12) In the medical judgment of the investigator, the subject had a psychiatric or psychological disorder that would interfere with the completion of the study, confound the study results, or pose patient risk;
13) Clinically significant abnormalities in clinical chemistry, hematology or urinalysis, including serum glutamic-oxaloacetic transaminase/aspartate aminotransferase (AST) or serum glutamic-pyruvic transaminase/alanine aminotransferase (ALT) ≧3.0 times the upper limit of the reference range or a serum creatinine >3.0 mg/dL at screening;
14) Medical condition, other than OA, that is not well controlled with treatment, or any clinically significant condition that would, in the opinion of the investigator, have precluded study participation or interfere with the assessment of pain and other symptoms of OA;
15) Unable to discontinue all formulations of prior analgesics (opioid and/or non-opioid) other than acetaminophen during the Washout Period of the study;
16) Received any investigational drug within 30 days prior to screening, or is scheduled to receive an investigational drug other than blinded study drug during the course of this study;
17) Documented history of, or currently active, seizure disorder (with the exception of febrile seizures);
18) Requires treatment with monoamine oxidase inhibitors (MAOIs);
19) Documented history of a medical condition that, in the opinion of the investigator, would compromise the subject's ability to swallow, absorb, metabolize, or excrete study drug, including (but not limited to) intractable nausea and/or vomiting and/or severe gastrointestinal narrowing (pathologic or iatrogenic);
20) Screening laboratory values show the presence of Hepatitis B surface antigen (HBs Ag), Hepatitis C antibody (HCV Ab), or active Hepatitis A immunoglobulin M (HAV IgM), (Inactive Hepatitis A results are permitted.);
21) Primary diagnosis of Functional Class IV OA;
22) History of spinal stenosis, severe herniated disc, tumors/infections of the spinal cord, metastasis, seronegative spondyloarthropathy, major trauma to L-S spine, back pain due to visceral disorder, or progressive neurological disorder;
23) Active gastrointestinal disease, with the exception of gastroesophageal reflux disease (GERD);
24) Surgical intervention to the back within six months of study entry or plans for surgical intervention while in the study;
25) Underwent an elective surgical procedure within eight weeks prior to screening, or is scheduled for an elective surgical procedure during the course of the study;
26) Incurred an injury at the target joint within 12 weeks prior to screening;
27) Documented history of prior disease (other than OA) and/or surgery at the target joint within the lust year prior to enrollment;
28) Documented history of sciatica, gout, pseudogout, and experienced flare within the last 2 years or has a history of Paget's disease;
29) Documented history of rheumatoid arthritis, uncontrolled inflammatory arthritis (e.g. psoriatic arthritis) or NSAID-dependent inflammatory arthritis;
30) Any chronic pain syndrome (i.e., fibromyalgia) that, in the investigator's opinion, would interfere with the assessment of pain and/or other symptoms of OA;
31) Received recent epidural or local corticosteroid injections in target joint within two months of screening, or target joint viscosupplementation within the past three months;
32) Received oral or intramuscular corticosteroids within the past 90 days. (Topical, nasal, and inhaled corticosteroids are permitted.);
33) Effective dose resulting from die Titration Phase of the study is <20 mg BID or >80 my BID;
34) Involved in an ongoing worker's compensation claim or litigation related to the target joint, or has settled a worker's compensation claim or disability claim related to the target joint within the past five years;
35) Considered by the investigator, for any reason, to be an unsuitable candidate to receive extended release morphine sulfate with naltrexone, including (but not limited to) the risk(s) in terms of precautions, warnings, and contraindications in the Investigator's Brochure for Kadian NT;
36) Historically non-responsive to morphine;
37) Previous allergy to acetaminophen; and/or,
38) History of severe impairment of pulmonary function, hypercarbia, hypoxia, chronic obstructive pulmonary disease, cor pulmonale, uncontrolled asthma, sleep apnea syndrome, or respiratory depression.

### 9. Efficacy Variables

The primary efficacy measure was the change from randomization baseline to the Visit Y+12 Weeks diary BPI score of average pain (daily scores of average pain averaged for each subject over a 7 day interval to obtain a weekly score). Continuous secondary efficacy variables include the following:
Diary BPI average pain averaged over the entire maintenance period;
In-clinic BPI;
Diary BPI worst, least, average and current pain (averaged over 7-day intervals to obtain weekly scores);
WOMAC Osteoarthritis Tndex Pain Subscale, Stiffness Subscale, Physical Function Subscale, and Composite Index;
MOS Sleep Scale subscale scores (sleep disturbance, snoring, awaken short of breath or with a headache, quantity of sleep, optimal sleep, sleep adequacy, and somnolence) and nine-item overall sleep problems index;
Beck Depression Inventory score; and,
Amount of rescue (pill counts summed over 7-day intervals to obtain weekly counts).

Categorical secondary efficacy variables include the following:
Patient Global Impression of Change (PGIC); and,
Responders at Week 12

### 10. Safety Endpoints

Safety was assessed based on AEs, clinical laboratory data, vital signs, and two measures of opioid withdrawal; SOWS and COWS.

### 11. Analysis Populations

Four subject analysis populations were defined as follows:
Intent-to-Treat (ITT) population: all subjects who are randomized into the Maintenance Phase of the study and take at least one dose of double-blind study medication after randomization.
Completers population: all subjects who complete the 12-week Maintenance Phase of the study without major protocol violations.
Safety population: all subjects who are administered any amount of double-blind study medication in the Maintenance Phase.
Titration Phase population: all subjects who are administered any amount of Kadian NT in the Titration Phase

Membership in the analysis populations were determined prior to unblinding. Subjects in the ITT population who were not in the Completers and Safety populations were summarized by reason for exclusion from the respective analysis population. In the event that a subject was randomized incorrectly or administered the incorrect study medication, analyses of the ITT and Completer populations was to be based on the assigned treatment, whereas all other analyses would be based on the actual treatment. Subjects whose assigned treatment depends on analysis population were identified.

### 12. Efficacy Analyses

### A. Primary Analysis:

The primary efficacy measure was changed front randomization baseline to the Visit Y+12 Weeks diary BPI score of average pain (daily scores of average pain will be averaged for each subject over a 7-day interval to obtain a weekly score). For subjects who complete the study, the final 7-day interval on study was used. The following imputation rules were used for subjects who prematurely discontinued from the study;
Screening baseline will be imputed for discontinuations due to adverse events. Screening baseline is defined as the in-clinic BPI obtained at Visit X. This imputation rule assigns no efficacy benefit to study drug when the subject discontinues for an adverse event;
If the results of the COWS questionnaire at discontinuation were worse than at randomization baseline (Visit Y) and indicated at least a moderate (score ≦13) level of withdrawal symptoms, the following imputation rules were used:
   Randomization baseline will be imputed for the placebo group. This imputation rule applies regardless of reason for discontinuation, and assigns full efficacy benefit to subjects in the placebo group who discontinue while experiencing at least moderate withdrawal symptoms.
   The weekly diary BPI average pain score during the last 7 days on study will be imputed for discontinuations in the Kadian NT group due to lack of efficacy or administrative reasons. Screening baseline will be imputed for discontinuations in the Kadian NT group due to adverse events. This imputation rule assigns a score that is worse than the randomization baseline score for subjects who repot at least moderate levels of withdrawal symptoms.
The weekly diary BPI average pain score during the last 7 days on study was imputed for discontinuations due to lack of efficacy or administrative reasons. This imputation rule assigned the actual pain reported at discontinuation, which for both study drugs tends to be worse than randomization baseline when open-label Kadian NT is administered but less severe than screening baseline.

Screening baseline is defined as the in-clinic BPI obtained at Visit X. Randomization baseline is defined as the diary BPI average pain score averaged over the last 7 days of the Titration Phase. If the diary BPI average pain score after randomization is missing for >3 days during the 7-day interval identified for analysis, the 7-day average will be considered missing and the above imputation rules will be used to estimate the missing value.

The primary statistical analysis was the analysis of covariance (ANCOVA) with treatment as a categorical factor and the randomization baseline score as covariate. The primary efficacy analysis population was the ITT population.

### B. Sensitivity Analysis

Three additional imputation methods will be examined as sensitivity analyses for the impact of opiate withdrawal on the primary efficacy variable of weekly diary BPI average pain score. For the first method, the randomization baseline will be imputed for all subjects (regardless of treatment group) who prematurely discontinue the study. For the second method in both treatment groups, the screening baseline will be imputed for subjects who discontinue for adverse events or lack of efficacy, and the randomization baseline will be imputed for subjects who discontinue for any other reason. For the third method, the screening baseline will be imputed for all subjects (regardless of treatment group) who prematurely discontinue the study. These will be supportive efficacy analyses of the primary endpoint and should be directionally consistent with the primary analysis. However, statistical significance and predetfined power >80% are not requited.

### D. Secondary Analyses

Continuous secondary efficacy variables include the following:
Diary BPI average pain averaged over the entire maintenance period;
In-clinic BPI;
Weekly diary BPI worst, least, and current pain (daily scores averaged over 7-day intervals to obtain weekly scores);
WOMAC Osteoarthritis Index Pain Subscale, Stiffness Subscale, Physical Function Subscale, and Composite Index;
MOS Sleep Scale subscale scores (sleep disturbance, snoring, awaken short of breath or with a headache, quantity of sleep, optimal sleep, sleep adequacy, and somnolence) and nine-item overall sleep problems index;
Beck Depression Inventory score; and
Amount of rescue (pill counts summed over 7-day intervals to obtain weekly counts);
   Categorical secondary efficacy variables include the following:
PGIC; and,
Responders at Week 12 based on in-clinic BPI.

Continuous secondary efficacy variables observed during the Titration Phase (in-clinic BPI and diary BPI worst, least, average, and current pain) were summarized at each visit in terms of descriptive statistics including the number of observations, mean, standard deviation, minimum, maximum, and quartiles. Actual values and change from Baseline to each visit and the final value prior to randomization or discontinuation were summarized. Only subjects with both a Baseline and a post-Baseline value during the titration phase were included in the change from Baseline analysis.

The proportion of subjects who were responders at Visit Y were summarized. Subjects who failed to qualify for randomization were considered non-responders. Subjects who completed Visit Y were defined as responders by a range of percent decreases from Visit X to Visit Y on the in-clinic 24-hour pain assessment. Response criteria was to range from 0% to 100% decreases (in increments of 10%). The proportion of responders was displayed graphically. The above analysis was conducted for the Titration Phase analysis population and only included visits occurring during the titration phase.

Continuous secondary efficacy variables observed during the Maintenance Phase were summarized at each visit in tenns of descriptive statistics by treatment. Actual values and change from Visit Y (except the Beck Depression Inventory score) were summarized. Change from Visit Y was compared between treatments at each visit using an ANCOVA with treatment as the factor and Visit Y value as the covariate. Change from Visit Y+2 weeks was compared between treatments at each subsequent visit using an ANCOVA with treatment as the factor and Visit Y+2 weeks value as the covariate. Only subjects with both a Visit Y or Visit Y+2 weeks value and a subsequent visit were included in the respective change from Visit Y or Visit Y+2 weeks analyses. In addition to analyzing the observed cases, missing observations were imputed based on the same logic as for the primary efficacy analysis.

The Maintenance Phase continuous secondary efficacy variables were analyzed using a mixed-effects repeated measures model. The response variable was the efficacy variable in question at each visit in the Maintenance Phase. The model included fixed-effects model terms for days on study, treatment, their interaction, and the Visit Y value of the variable in question as a covariate. The covariance structure with the largest value for Schwarz's Bayesian Criterion (BIC) from PROC MIXED was employed. Missing data was not be imputed in this analysis.

The cumulative proportion of subjects who were responders at Visit Y+12 Weeks of the Maintenance Phase was summarized with the method of Farrar (2006). All subjects with both a Baseline and at least one Maintenance Phase in-clinic 24 hour BPI assessment were included in the analysis. Subjects were defined as responders by the percent decrease from Visit X to Visit Y+12 Weeks on the in-clinic 24-hour pain assessment. Subjects discontinued from the study before Visit Y+12 Weeks were considered non-responders. Treatment differences in the proportion of subjects who report at least 20%, 30%, 40%, and 50% improvement were assessed with Fisher's exact test.

Categorical secondary efficacy variables (e.g., the PGIC) were summarized at each visit in terms of frequencies and percentages, by treatment. These were compared between treatments using a CMB test with row mean scores. In addition to analyzing the observed cases, missing observations were imputed using the method described for the continuous variables. The above analysis was conducted for the ITT and Completers analysis populations.

### 13. Safety Analyses

Safety was assessed based on AEs, laboratory values, vital signs, and two measures of opioid withdrawal: SOWS and COWS.

### A. Titration Phase:

The number and percentage of subjects with AEs were displayed by body system and preferred term using the Medical Dictionary for Regulatory Activities (MedDRA). Summaries in terms of severity and relationship to study drug were also provided. Serious Adverse Events (SAEs) were summarized separately in a similar manner. Subject listings of AEs causing discontinuation of study medication and SAEs were produced. These analyses were performed based on AEs with a start date during the Titration Phase.

Vital signs will be summarized at each visit in terms of descriptive statistics including the mean, standard deviation, minimum, maximum, and quartiles. Actual values and change from Baseline (Visit X) to each visit and the final value prior to randomization or discontinuation were summarized. Only subjects with both a Baseline and a post-Baseline value during the titration phase were included in the change from Baseline analysis. The above analyses will be conducted for the Titration Phase analysis population and will only include visits occurring during the Titration Phase.

### B. Maintenance Phase:

The number and percentage of subjects with AEs were displayed by body system and preferred term using MedDRA, by treatment. Summaries in terms of severity and relationship to study drug were also provided. SAEs were summarized separately in a similar manner. Subject listings of AEs causing discontinuation of study medication and SAEs were produced. These analyses were performed based on AEs with a start date on or after the date of the first dose of randomized study drug and repeated for AEs with a start date on or after the date of the Visit Y+2 weeks visit. The frequencies of AEs among the treatment groups were compared using Fisher's exact test.

Vital signs will be summarized at each visit in terms of descriptive statistics by treatment. Actual values, change from Visit Y, and change from Visit Y+2 weeks were summarized. Change from Visit Y were compared between treatments at each visit using an ANCOVA with treatment as the factor and Visit Y value as the covariate. Change from Visit Y+2 weeks was compared between treatments at each subsequent visit using an ANCOVA with treatment as the factor and Visit Y+2 weeks value as the covariate. Only subjects with both a Visit Y or Visit Y+2 weeks value and a subsequent visit were included in the respective change from Visit Y or Visit Y+2 weeks analyses. The vital signs were also be categorized according to Potentially Clinically Significant (PCS) criteria. The frequency and percentage of subjects with at least one value during the Maintenance Phase that meets the PCS criteria were summarized for the two treatment groups.

Quantitative laboratory test results were summarized at Visit Y+12 Weeks in terms of descriptive statistics, by treatment. Actual values and change from Visit Y was summarized. Change from Visit Y was compared between treatments at each visit using an ANCOVA with treatment as the factor and Visit Y value as the covariate. Only subjects with both a Visit Y and a subsequent visit were included in the respective change from Visit Y analyses.

The quantitative laboratory test results were also categorized according to Potentially Clinically Significant (PCS) criteria. The frequency and percentage of subjects with at least one value during the Maintenance Phase that meets the PCS criteria were summarized for the two treatment groups. For qualitative laboratory tests, the number and percentage of subjects in each category were produced for each treatment at Visit Y+12 Weeks. For all laboratory tests, a shift table was produced summarizing changes from normal (at Baseline) to abnormal and vice-versa. Only subjects with both a Baseline and a post-Baseline value were included in the change from Baseline analysis.

COWS were summarized in terms of descriptive statistics by treatment. Actual values and change from Visit Y to Visit Y+1 were summarized for subjects whose dose of Kadian NT was ≦30 mg at randomization. For subjects whose dose of Kadian NT was >80 mg at randomization, actual values and change from Visit Y to Visit Y+2 weeks was summarized.

SOWS were summarized in terms of descriptive statistics by treatment. Actual values and change from Visit Y to the most severe score on Days 5-7 were summarized for subjects whose dose of Kadian NT was <80 mg at randomization. For subjects whose dose of Kadian NT was >80 mg at randomization, actual values and change from Visit Y to the most severe score on Days 12-14 were summarized. The first three evaluations following study drug discontinuation were used for subjects who discontinued before the specified range of study dates.

### 14. Sample Size Considerations

The sample size calculation was based on the primary efficacy analysis. The null hypothesis was that there was no treatment group difference for the primary efficacy analysis and the alternative hypothesis was that a treatment group difference does exist. No adjustment for multiple analyses was made because the primary efficacy endpoint and analysis were specified. A Type I error of 0.05 for a 2-tailed test with at least 90% power was specified. An effect size (mean treatment group difference divided by the pooled standard deviation) of 0.33 was assumed for the primary efficacy analysis. Given these assumptions, a sample size of 200 subjects randomized to each treatment group was required to obtain at least 90% power.

A summary of the procedures described above is provided in the following table:

Schedule of Observations and Procedures

| | | | | Titration Phase | Maintenance Phase² | | | | | | |
|---|---|---|---|---|---|---|---|---|---|---|---|
| | | | | (Weekly | (12 Weeks Total. Visits | | | | | | |
| | | Wash- | | visits up to 6 | every week for 2 weeks, then | | | | | | |
| | | out | Base- | weeks total) | every 2 weeks up to 12 weeks) | | | | | | |
| | Screening | Period | line¹ | Visits X + | | | Visits | Visits | | | |
| | Visit | Day 1 to | Visit | 1, 2, 3, | | Visit | Y + 2, | Y + 4, | Visits | Post-Tx | |
| | Day −14 | Day 7 of | Visit X | 4, 5 & | Visit | Y + 1 | 6, & 10 | & 8 | Y + 12 | Follow- | Early |
| | to Day −1 | Screening | (Day 0) | 6 Weeks | Y | Week | Weeks | Weeks | Weeks | Up | Termination |
| Informed consent | X | | | | | | | | | | |
| Inclusion/exclusion | X | | X | | | | | | | | |
| Medical history incl. | X | | | | | | | | | | |
| chronic pain history | | | | | | | | | | | |
| 12-lead ECG | X | | | | | | | | | | |
| Urine drug-screen | X | | X | | | | | | | | |
| Physical examination | X | | | | | | | | X | | X |
| and weight | | | | | | | | | | | |
| Height, Weight, and | X | | | | | | | | | | |
| Body Mass Index | | | | | | | | | | | |
| Vital signs | X | | X | X | X | X | X | X | X | X | X |
| Clinical laboratory | X | | X | | | | | | X | | X |
| tests | | | | | | | | | | | |
| Urine pregnancy test | X | | X | | | | | | | | |
| Experience minimum | | X | | | | | | | | | |
| pain flare score³ | | | | | | | | | | | |
| Dispense electronic | X | | X | X | X | X | X | X | | | |
| diary | | | | | | | | | | | |
| Dispense study drug | X | | X | X | X | | X | X | X | | X |
| and/or rescue | | | | | | | | | | | |
| medication | | | | | | | | | | | |
| Collect and Review | | | X | X | X | X | X | X | X | | X |
| electronic diary | | | | | | | | | | | |
| Collect study drug | | | X | X | X | X | X | X | X | X | X |
| and/or rescue | | | | | | | | | | | |
| medication | | | | | | | | | | | |
| Adverse events | | | X | X | X | X | X | X | X | X | X |
| Concomitant | | | X | X | X | X | X | X | X | X | X |
| medications | | | | | | | | | | | |
| Beck Depression | | | X | | | | | X | X | | X⁴ |
| Inventory | | | | | | | | | | | |
| MOS Sleep Scale | | | X | | X | | | X | X | | X⁴ |
| In-clinic pain | | | X | X | X | X | X | X | X | | X |
| assessment (BPI) | | | | | | | | | | | |
| WOMAC | | | X | | X | | X | X | X | | X⁴ |
| Osteoarthritis Index | | | | | | | | | | | |
| Brief Pain Inventory | | | X | X | X | X | X | X | X | | X |
| (BPI)⁵ | | | | | | | | | | | |
| Patient Global | | | | | X | | X | X | X | | X |
| Impression of Change | | | | | | | | | | | |
| (PGIC) | | | | | | | | | | | |
| Clinical Opiate | | | | | X | X | X⁶ | | X | | X |
| Withdrawal Scale | | | | | | | | | | | |
| (COWS) | | | | | | | | | | | |
| Subjective Opiate | | | | | X | X | X⁶ | | | | |
| Withdrawal Scale | | | | | | | | | | | |
| (SOWS)⁷ | | | | | | | | | | | |

¹Visit X = Baseline Visit (Day 0).
²Visit Y = first day of the Maintenance Phase.
³Minimum Pain Flare Score = average 24 hour pain intensity of ≧5 on the 11-point BPI scale.
⁴Subjects who prematurely withdraw from the Titration Phase of the study should not complete this assessment.
⁵BPI included in daily electronic diary completion only.
⁶Performed at the Visit Y + 2 Weeks only.
⁷Included in thc daily electronic diary completion; completed daily for the first two weeks of the Maintenance Phase.

### B. Results

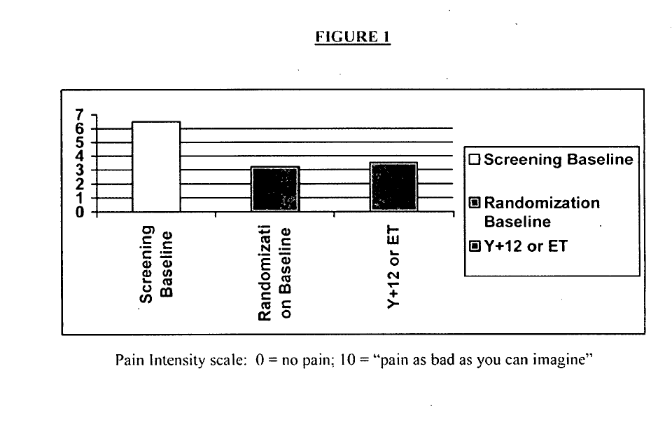

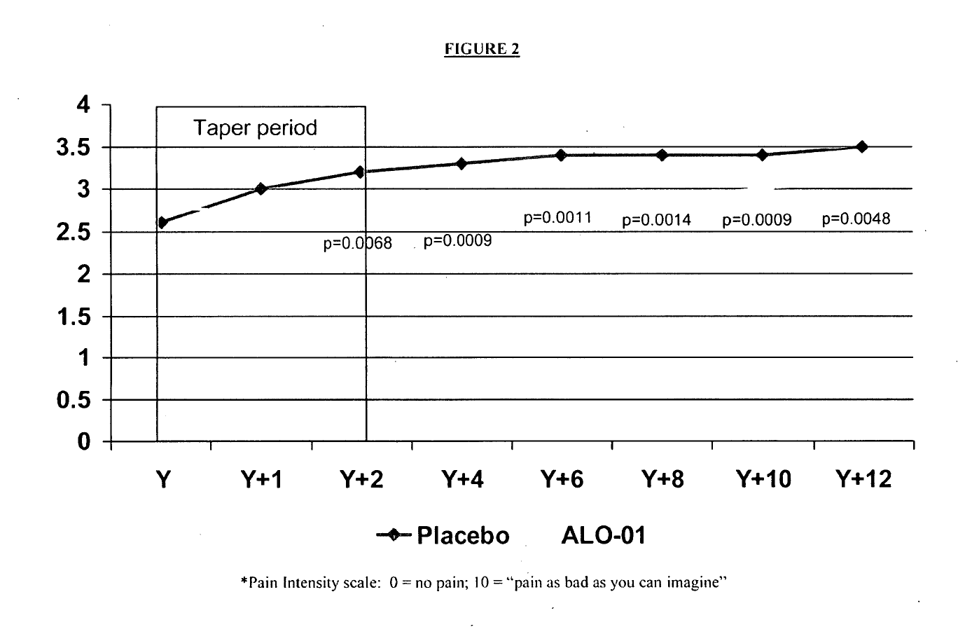

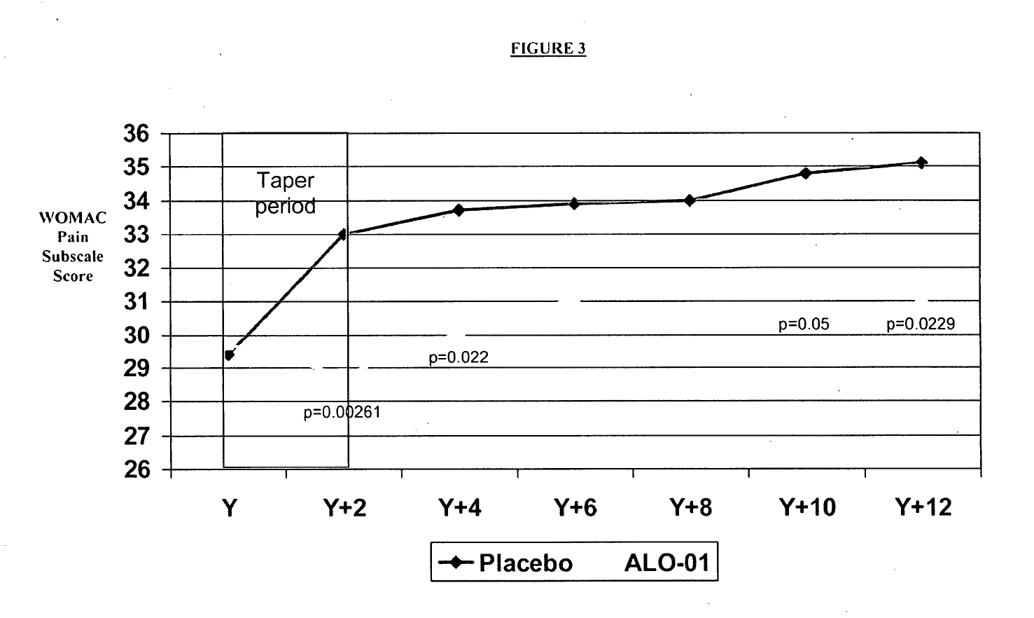

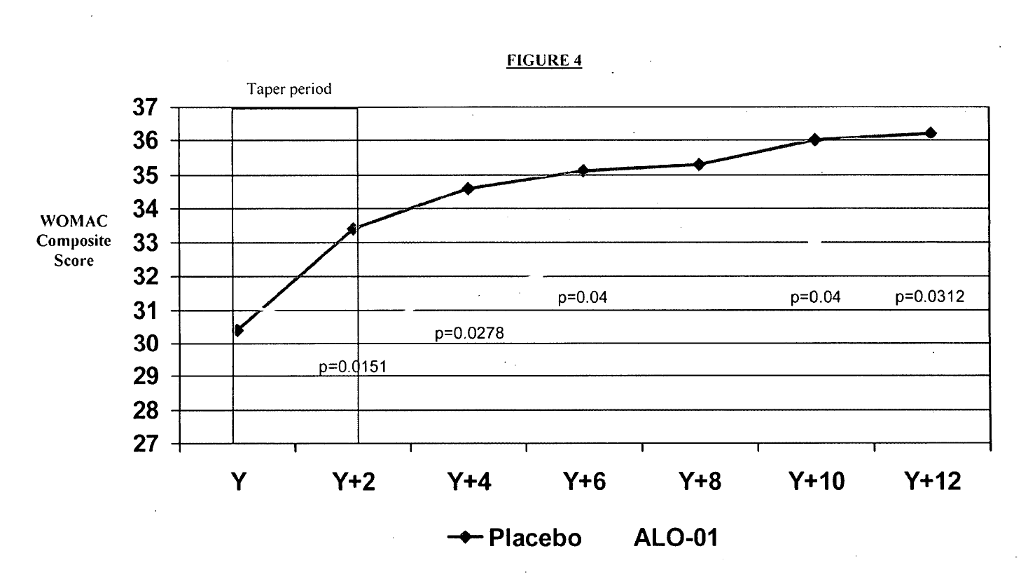

As shown in the following tables, treatment with Kadian NT provides pain relief to patients for up to twelve weeks in a manner that is more efficacious than placebo. The superiority of Kadian NT over placebo was confirmed using BPI scores and the WOMAC Osteoarthritis index. A summary of the data is also shown in FIGS. 1, 2, 3 and 4. This data indicates that the affects of morphine in this population is not negatively affected by the concomitant administration of both morphine and naltrexone in an intact dosage form (Kadian NT).

11.4.2.2 Change in Weekly Diary BPI Average Pain Score
at Week Y + 12 (Primary Endpoint) ITT Population

| | | Kadian NT | Placebo | |
|---|---|---|---|---|
| Visit | Statistic | N = 170 | N = 173 | P-value [a] |
| Baseline | N | 170 | 173 | |
| | Mean | 3.3 | 3.2 | |
| | SD | 1.30 | 1.07 | |
| | Q1 | 2.4 | 2.6 | |
| | Median | 3.4 | 3.3 | |
| | Q3 | 4.1 | 4.0 | |
| | Min/Max | 0/9 | 0/6 | |
| Week Y + 12 [b] | N | 170 | 173 | |
| | Mean | 3.1 | 3.5 | |
| | SD | 1.99 | 2.13 | |
| | Q1 | 1.4 | 2.0 | |
| | Median | 3.0 | 3.0 | |
| | Q3 | 4.1 | 5.0 | |
| | Min/Max | 0/10 | 0/9 | |
| Change from Baseline | N | 170 | 173 | |
| | Mean | −0.2 | 0.3 | 0.0445 |
| | SD | 1.94 | 2.05 | |
| | Q1 | −1.0 | −1.1 | |
| | Median | −0.1 | 0.1 | |
| | Q3 | 0.9 | 1.4 | |
| | Min/Max | −6/6 | −4/6 | |

[a] Difference between treatment groups evaluated by ANCOVA with treatment as categorical factor and randomization baseline score as covariate.
[b] Primary endpoint imputation algorithm used.

11.4.3 Change in Weekly Diary BPI Average Pain Score
at Week Y + 12 (Sensitivity Analyses) ITT Population

| | | Kadian NT | Placebo | |
|---|---|---|---|---|
| Visit | Statistic | N = 170 | N = 173 | P-value [a] |
| Baseline | N | 170 | 173 | |
| | Mean | 3.3 | 3.2 | |
| | SD | 1.30 | 1.07 | |
| | Q1 | 2.4 | 2.6 | |
| | Median | 3.4 | 3.3 | |
| | Q3 | 4.1 | 4.0 | |
| | Min/Max | 0/9 | 0/6 | |

Imputation Method: Randomization Baseline

| | | | | |
|---|---|---|---|---|
| Week Y + 12 | N | 170 | 173 | |
| | Mean | 2.9 | 3.1 | |
| | SD | 1.59 | 1.58 | |
| | Q1 | 1.7 | 2.0 | |
| | Median | 3.0 | 3.0 | |
| | Q3 | 4.0 | 4.0 | |
| | Min/Max | 0/9 | 0/8 | |
| Change from Baseline | N | 170 | 173 | |
| | Mean | −0.4 | −0.2 | 0.1223 |
| | SD | 1.34 | 1.32 | |
| | Q1 | −1.0 | −0.6 | |
| | Median | 0.0 | 0.0 | |
| | Q3 | 0.0 | 0.0 | |
| | Min/Max | −4/5 | −4/4 | |

[a] Difference between treatment groups evaluated by ANCOVA with treatment as categorical factor and randomization baseline score as covariate.
[b] Baseline scores are only presented for patients who have non-missing values for Week Y + 12 score.

11.4.3 Change in Weekly Diary BPI Average Pain Score
at Week Y + 12 (Sensitivity Analyses) ITT Population

| | | | | |
|---|---|---|---|---|
| | | Kadian NT | Placebo | |
| Visit | Statistic | N = 170 | N = 173 | P-value [a] |

Imputation Method: Screening or Randomization Baseline

| | | | | |
|---|---|---|---|---|
| Week Y + 12 | N | 170 | 173 | |
| | Mean | 3.3 | 3.9 | |
| | SD | 2.13 | 2.38 | |
| | Q1 | 1.9 | 2.0 | |
| | Median | 3.0 | 3.6 | |
| | Q3 | 4.3 | 5.3 | |
| | Min/Max | 0/10 | 0/10 | |
| Change from Baseline | N | 170 | 173 | |
| | Mean | 0.0 | 0.7 | 0.0051 |
| | SD | 1.91 | 2.17 | |
| | Q1 | −1.0 | −0.6 | |
| | Median | 0.0 | 0.0 | |
| | Q3 | 0.7 | 2.0 | |
| | Min/Max | −4/6 | −4/7 | |

[a] Difference between treatment groups evaluated by ANCOVA with treatment as categorical factor and randomization baseline score as covariate.
[b] Baseline scores are only presented for patients who have non-missing values for Week Y + 12 score.

11.4.3 Change in Weekly Diary BPI Average Pain Score
at Week Y + 12 (Sensitivity Analyses) ITT Population

| | | | | |
|---|---|---|---|---|
| | | Kadian NT | Placebo | |
| Visit | Statistic | N = 170 | N = 173 | P-value [a] |

Imputation Method: Screening Baseline

| | | | | |
|---|---|---|---|---|
| Week Y + 12 | N | 170 | 173 | |
| | Mean | 3.9 | 4.3 | |
| | SD | 2.54 | 2.49 | |
| | Q1 | 1.9 | 2.3 | |
| | Median | 3.4 | 4.0 | |
| | Q3 | 6.0 | 6.0 | |
| | Min/Max | 0/10 | 0/10 | |
| Change from Baseline | N | 170 | 173 | |
| | Mean | 0.6 | 1.1 | 0.0489 |
| | SD | 2.31 | 2.37 | |
| | Q1 | −1.0 | −0.6 | |
| | Median | 0.4 | 1.0 | |
| | Q3 | 2.0 | 2.9 | |
| | Min/Max | −4/9 | −4/7 | |

[a] Difference between treatment groups evaluated by ANCOVA with treatment as categorical factor and randomization baseline score as covariate.
[b] Baseline scores are only presented for patients who have non-missing values for Week Y + 12 score.

TABLE 11.4.4
BPI Diary Average Pain Score—Maintenance Phase Imputed Values
ITT Population

| | | Placebo | | | | | Kadian NT | | | | | | |
|---|---|---|---|---|---|---|---|---|---|---|---|---|---|
| | | N = 173 | | | | | N = 170 | | | | | P-value [c] | |
| | | | | Change | | Change | | | Change | | Change | Change | Change |
| | | Actual | Visit | From | Visit | From | Actual | Visit | From | Visit | From | From | From |
| | | Value | Y | Visit | Y + 2 | Visit | Value | Y | Visit | Y + 2 | Visit | Visit | Visit |
| Visit | Statistic | [a] | [b] | Y | [b] | Y + 2 | [a] | [b] | Y | [b] | Y + 2 | Y | Y + 2 |
| Y | n | 173 | | | | | 170 | | | | | | |
| | Mean | 2.6 | | | | | 2.8 | | | | | | |
| | Std Dev | 1.23 | | | | | 1.34 | | | | | | |
| | Q1 | 2.0 | | | | | 2.0 | | | | | | |
| | Median | 3.0 | | | | | 3.0 | | | | | | |
| | Q3 | 3.5 | | | | | 4.0 | | | | | | |
| | Min/Max | 0/6 | | | | | 0/6 | | | | | | |
| Y + 1 | n | 173 | 173 | 173 | | | 170 | 170 | 170 | | | 0.0703 | |
| | Mean | 3.0 | 2.6 | 0.3 | | | 2.7 | 2.8 | −0.0 | | | | |
| | Std Dev | 1.37 | 1.23 | 1.14 | | | 1.39 | 1.34 | 0.87 | | | | |
| | Q1 | 2.0 | 2.0 | −0.2 | | | 1.8 | 2.0 | −0.5 | | | | |
| | Median | 3.0 | 3.0 | 0.2 | | | 2.8 | 3.0 | 0.0 | | | | |
| | Q3 | 4.0 | 3.5 | 0.8 | | | 3.8 | 4.0 | 0.5 | | | | |
| | Min/Max | 0/7 | 0/6 | −4/5 | | | 0/7 | 0/6 | −3/2 | | | | |
| Y + 2 | n | 173 | 173 | 173 | | | 170 | 170 | 170 | | | 0.0068 | |
| | Mean | 3.2 | 2.6 | 0.5 | | | 2.8 | 2.8 | 0.0 | | | | |
| | Std Dev | 1.72 | 1.23 | 1.58 | | | 1.57 | 1.34 | 1.32 | | | | |
| | Q1 | 2.0 | 2.0 | −0.3 | | | 1.6 | 2.0 | −0.7 | | | | |
| | Median | 3.0 | 3.0 | 0.2 | | | 2.9 | 3.0 | 0.0 | | | | |
| | Q3 | 4.2 | 3.5 | 1.3 | | | 4.0 | 4.0 | 0.4 | | | | |
| | Min/Max | 0/8 | 0/6 | −5/5 | | | 0/7 | 0/6 | −4/7 | | | | |

[a] Actual values are derived weekly averages of daily average pain scores.
[b] Visits Y and Y+ 2 values for only those subjects present at Visit Y + x are shown in this column.
[c] Differences between treatments in Change from Visit Y were assessed using mixed model random effects ANCOVA with contrasts for by-visit treatment comparisons.

TABLE 11.4.4
BPI Diary Average Pain Score—Maintenance Phase Imputed Values (ITT Population)

| | | Placebo | | | | | Kadian NT | | | | | | |
|---|---|---|---|---|---|---|---|---|---|---|---|---|---|
| | | N = 173 | | | | | N = 170 | | | | | P-value [c] | |
| | | | | Change | | Change | | | Change | | Change | Change | Change |
| | | Actual | Visit | From | Visit | From | Actual | Visit | From | Visit | From | From | From |
| | | Value | Y | Visit | Y + 2 | Visit | Value | Y | Visit | Y + 2 | Visit | Visit | Visit |
| Visit | Statistic | [a] | [b] | Y | [b] | Y + 2 | [a] | [b] | Y | [b] | Y + 2 | Y | Y + 2 |
| Y + 4 | n | 173 | 173 | 173 | 173 | 170 | 170 | 170 | 170 | 170 | 170 | 0.0009 | 0.2795 |
| | Mean | 3.3 | 2.6 | 0.7 | 3.2 | 0.1 | 2.8 | 2.8 | 0.1 | 2.8 | 0.0 | | |
| | Std Dev | 1.90 | 1.23 | 1.80 | 1.72 | 1.04 | 1.70 | 1.34 | 1.62 | 1.57 | 1.04 | | |
| | Q1 | 2.0 | 2.0 | −0.4 | 2.0 | −0.2 | 1.6 | 2.0 | −0.9 | 1.6 | −0.3 | | |
| | Median | 3.0 | 3.0 | 0.4 | 3.0 | 0.0 | 2.8 | 3.0 | 0.0 | 2.9 | 0.0 | | |
| | Q3 | 4.7 | 3.5 | 1.7 | 4.2 | 0.5 | 4.0 | 4.0 | 0.7 | 4.0 | 0.1 | | |
| | Min/Max | 0/9 | 0/6 | −5/6 | 0/8 | −4/5 | 0/8 | 0/6 | −6/7 | 0/7 | −2/8 | | |
| Y + 6 | n | 173 | 173 | 173 | 173 | 173 | 170 | 170 | 170 | 170 | 170 | 0.0011 | 0.2988 |
| | Mean | 3.4 | 2.6 | 0.7 | 3.2 | 0.2 | 2.9 | 2.8 | 0.1 | 2.8 | 0.1 | | |
| | Std Dev | 1.95 | 1.23 | 1.81 | 1.72 | 1.22 | 1.81 | 1.34 | 1.79 | 1.57 | 1.26 | | |
| | Q1 | 2.0 | 2.0 | −0.4 | 2.0 | −0.2 | 1.6 | 2.0 | −0.9 | 1.6 | −0.3 | | |
| | Median | 3.0 | 3.0 | 0.4 | 3.0 | 0.0 | 2.8 | 3.0 | 0.0 | 2.9 | 0.0 | | |
| | Q3 | 4.9 | 3.5 | 1.8 | 4.2 | 0.6 | 4.0 | 4.0 | 1.0 | 4.0 | 0.1 | | |
| | Min/Max | 0/9 | 0/6 | −5/6 | 0/8 | −4/5 | 0/8 | 0/6 | −4/7 | 0/7 | −2/8 | | |
| Y + 8 | n | 173 | 173 | 173 | 173 | 173 | 170 | 170 | 170 | 170 | 170 | 0.0014 | 0.3473 |
| | Mean | 3.4 | 2.6 | 0.7 | 3.2 | 0.2 | 2.9 | 2.8 | 0.2 | 2.8 | 0.1 | | |
| | Std Dev | 1.96 | 1.23 | 1.84 | 1.72 | 1.24 | 1.79 | 1.34 | 1.78 | 1.57 | 1.29 | | |
| | Q1 | 2.0 | 2.0 | −0.3 | 2.0 | −0.2 | 1.6 | 2.0 | −0.9 | 1.6 | −0.4 | | |
| | Median | 3.1 | 3.0 | 0.5 | 3.0 | 0.0 | 2.9 | 3.0 | 0.0 | 2.9 | 0.0 | | |
| | Q3 | 4.9 | 3.5 | 2.0 | 4.2 | 0.6 | 4.0 | 4.0 | 1.0 | 4.0 | 0.2 | | |
| | Min/Max | 0/9 | 0/6 | −5/6 | 0/8 | −3/5 | 0/8 | 0/6 | −4/7 | 0/7 | −3/8 | | |

[a] Actual values are derived weekly averages of daily average pain scores.
[b] Visits Y and Y + 2 values for only those subjects present at Visit Y + x are shown in this column.
[c] Differences between treatments in Change from Visit Y were assessed using mixed model random effects ANCOVA with contrasts for by-visit treatment comparisons.

TABLE 11.4.4
BPI Diary Average Pain Score Maintenance—Phase Imputed Values (ITT Population)

| | | Placebo | | | | | Kadian NT | | | | | | |
|---|---|---|---|---|---|---|---|---|---|---|---|---|---|
| | | N = 173 | | | | | N = 170 | | | | | P-value [c] | |
| | | | | Change | | Change | | | Change | | Change | Change | Change |
| | | Actual | Visit | From | Visit | From | Actual | Visit | From | Visit | From | From | From |
| | | Value | Y | Visit | Y + 2 | Visit | Value | Y | Visit | Y + 2 | Visit | Visit | Visit |
| Visit | Statistic | [a] | [b] | Y | [b] | Y + 2 | [a] | [b] | Y | [b] | Y + 2 | Y | Y + 2 |
| Y + 10 | n | 173 | 173 | 173 | 173 | 173 | 170 | 170 | 170 | 170 | 170 | 0.0009 | 0.2795 |
| | Mean | 3.4 | 2.6 | 0.8 | 3.2 | 0.3 | 3.0 | 2.8 | 0.2 | 2.8 | 0.2 | | |
| | Std Dev | 2.03 | 1.23 | 1.93 | 1.72 | 1.34 | 1.95 | 1.34 | 1.89 | 1.57 | 1.52 | | |
| | Q1 | 2.0 | 2.0 | −0.4 | 2.0 | −0.3 | 1.4 | 2.0 | −0.9 | 1.6 | −0.4 | | |
| | Median | 3.0 | 3.0 | 0.6 | 3.0 | 0.0 | 2.9 | 3.0 | 0.0 | 2.9 | 0.0 | | |
| | Q3 | 5.0 | 3.5 | 2.0 | 4.2 | 0.7 | 4.0 | 4.0 | 1.0 | 4.0 | 0.4 | | |
| | Min/Max | 0/9 | 0/6 | −5/6 | 0/8 | −3/5 | 0/1 | 0/6 | −4/7 | 0/7 | 4/8 | | |
| Y + 12 | n | 173 | 173 | 173 | 173 | 173 | 170 | 170 | 170 | 170 | 170 | 0.0048 | 0.6291 |
| | Mean | 3.5 | 2.6 | 0.8 | 3.2 | 0.3 | 3.1 | 2.8 | 0.3 | 2.8 | 0.3 | | |
| | Std Dev | 2.04 | 1.23 | 1.94 | 1.72 | 1.36 | 1.99 | 1.34 | 1.91 | 1.57 | 1.63 | | |
| | Q1 | 2.0 | 2.0 | −0.3 | 2.0 | −0.3 | 1.3 | 2.0 | −0.9 | 1.6 | −0.4 | | |
| | Median | 3.0 | 3.0 | 0.6 | 3.0 | 0.0 | 3.0 | 3.0 | 0.0 | 2.9 | 0.0 | | |
| | Q3 | 5.0 | 3.5 | 2.0 | 4.2 | 0.7 | 4.3 | 4.0 | 1.1 | 4.0 | 0.6 | | |
| | Min/Max | 0/9 | 0/6 | −5/6 | 0/8 | −3/6 | 0/1 | 0/6 | −4/7 | 0/7 | −5/8 | | |
| Average | n | 173 | 173 | 173 | 173 | 170 | 170 | 170 | 170 | | | 0.0017 | |
| Of All | Mean | 3.3 | 2.6 | 0.7 | 3.2 | 2.9 | 2.8 | 0.1 | 2.8 | | | | |
| Maint. | Std Dev | 1.72 | 1.23 | 1.58 | 1.72 | 1.55 | 1.34 | 1.41 | 1.57 | | | | |
| BPI | Q1 | 2.0 | 2.0 | −0.3 | 2.0 | 1.8 | 2.0 | −0.7 | 1.6 | | | | |
| Avg. | Median | 3.2 | 3.0 | 0.4 | 3.0 | 2.8 | 3.0 | 0.0 | 2.9 | | | | |
| Pain | Q3 | 4.6 | 3.5 | 1.7 | 4.2 | 4.0 | 4.0 | 0.9 | 4.0 | | | | |
| | Min/Max | 0/8 | 0/6 | −5/5 | 0/8 | 0/7 | 0/6 | −4/6 | 0/7 | | | | |

[a] Actual values are derived weekly averages of daily average pain scores.
[b] Visits Y and Y+ 2 values for only those subjects present at Visit Y + x are shown in this column.
[c] Differences between treatments in Change from Visit Y were assessed using mixed model random effects ANCOVA with contrasts for by-visit treatment comparisons.

TABLE 11.4.16
WOMAC Osteoarthritis Index—Maintenance Phase Imputed Values
ITT Population
WOMAC Composite Score

| | | Placebo | | | | | Kadian NT | | | | | | |
|---|---|---|---|---|---|---|---|---|---|---|---|---|---|
| | | N = 173 | | | | | N = 170 | | | | | P-value [c] | |
| | | | | Change | | Change | | | Change | | Change | Change | Change |
| | | Actual | Visit | From | Visit | From | Actual | Visit | From | Visit | From | From | From |
| | | Value | Y | Visit | Y + 2 | Visit | Value | Y | Visit | Y + 2 | Visit | Visit | Visit |
| Visit | Statistic | [a] | [b] | Y | [b] | Y + 2 | [a] | [b] | Y | [b] | Y + 2 | Y | Y + 2 |
| Y | n | 1.73 | | | | | 170 | | | | | | |
| | Mean | 30.4 | | | | | 31.2 | | | | | | |
| | Std Dv | 15.41 | | | | | 15.26 | | | | | | |
| | Q1 | 18.5 | | | | | 21.8 | | | | | | |
| | Median | 28.7 | | | | | 30.3 | | | | | | |
| | Q3 | 40.4 | | | | | 41.5 | | | | | | |
| | Min/Max | 0/74 | | | | | 0/79 | | | | | | |
| Y + 2 | n | 173 | 173 | 173 | | | 170 | 170 | 170 | | | 0.0151 | |
| | Mean | 33.4 | 30.4 | 3.0 | | | 30.4 | 31.2 | −0.8 | | | | |
| | Std Dev | 15.72 | 15.41 | 13.35 | | | 18.27 | 15.26 | 15.19 | | | | |
| | Q1 | 23.9 | 18.5 | −4.3 | | | 17.5 | 21.8 | −8.9 | | | | |
| | Median | 32.0 | 28.7 | 0.0 | | | 27.7 | 30.3 | 0.0 | | | | |
| | Q3 | 45.2 | 40.4 | 10.1 | | | 40.9 | 41.5 | 3.3 | | | | |
| | Min/Max | 0/75 | 0/74 | −24/44 | | | 0/78 | 0/79 | −54/63 | | | | |
| Y + 4 | n | 173 | 173 | 173 | 173 | 173 | 170 | 170 | 170 | 170 | 170 | 0.0278 | 0.7182 |
| | Mean | 34.6 | 30.4 | 4.2 | 33.4 | 1.2 | 31.6 | 31.2 | 0.4 | 30.4 | 1.2 | | |
| | Std Dev | 17.53 | 15.41 | 15.31 | 15.72 | 10.26 | 18.07 | 15.26 | 15.80 | 18.27 | 10.60 | | |
| | Q1 | 22.4 | 18.5 | −4.8 | 23.9 | −2.6 | 18.1 | 21.8 | −6.8 | 17.5 | −1.6 | | |
| | Median | 33.5 | 28.7 | 0.0 | 32.0 | 0.0 | 30.8 | 30.3 | 0.0 | 27.7 | 0.0 | | |
| | Q3 | 48.4 | 40.4 | 12.4 | 45.2 | 5.4 | 42.6 | 41.5 | 5.5 | 40.9 | 4.3 | | |
| | Min/Max | 0/84 | 0/74 | −29/54 | 0/75 | −39/39 | 0/78 | 0/79 | −58/63 | 0/78 | −44/52 | | |

[a] Visits Y and Y + 2 values for only those subjects present at Visit Y + x are shown in this column.
[b] Differences between treatments in Change from Visit Y assessed using ANCOVA with treatment as a categorical Factor and Visit Y or Y + 2 value as covariate.

TABLE 11.4.16
WOMAC Osteoarthritis Index—Maintenance Phase Imputed Values
ITT Population
WOMAC Composite Score

| | | Placebo | | | | | Kadian NT | | | | | | |
|---|---|---|---|---|---|---|---|---|---|---|---|---|---|
| | | N = 173 | | | | | N = 170 | | | | | P -value [c] | |
| | | | | Change | | Change | | | Change | | Change | Change | Change |
| | | Actual | Visit | From | Visit | From | Actual | Visit | From | Visit | From | From | From |
| | | Value | Y | Visit | Y + 2 | Visit | Value | Y | Visit | Y + 2 | Visit | Visit | Visit |
| Visit | Statistic | [a] | [b] | Y | [b] | Y + 2 | [a] | [b] | Y | [b] | Y + 2 | Y | Y + 2 |
| Y + 6 | n | 173 | 173 | 173 | 173 | 173 | 170 | 170 | 170 | 170 | 170 | 0.0411 | 0.8379 |
| | Mean | 35.1 | 30.4 | 4.7 | 33.4 | 1.7 | 32.3 | 31.2 | 1.1 | 30.4 | 1.9 | | |
| | Std Dev | 17.62 | 15.41 | 15.34 | 15.72 | 10.71 | 18.81 | 15.26 | 16.49 | 18.27 | 11.70 | | |
| | Q1 | 23.9 | 18.5 | −3.6 | 23.9 | −2.6 | 19.2 | 21.8 | −7.4 | 17.5 | −2.1 | | |
| | Median | 35.8 | 28.7 | 0.5 | 32.0 | 0.0 | 31.4 | 30.3 | 0.0 | 27.7 | 0.0 | | |
| | Q3 | 48.5 | 40.4 | 13.1 | 45.2 | 7.4 | 42.7 | 41.5 | 9.5 | 40.9 | 6.9 | | |
| | Min/Max | 0/92 | 0/74 | −29/54 | 0/75 | −39/39 | 0/86 | 0/79 | −55/63 | 0/78 | −45/52 | | |
| Y + 8 | n | 173 | 173 | 173 | 173 | 173 | 170 | 170 | 170 | 170 | 170 | 0.0753 | 0.9619 |
| | Mean | 35.3 | 30 | 4.8 | 33.4 | 1.9 | 32.7 | 31.2 | 1.5 | 30.4 | 2.3 | | |
| | Std Dev | 18.38 | 15.41 | 16.03 | 15.72 | 12.36 | 19.92 | 15.26 | 17.79 | 18.27 | 13.74 | | |
| | Q1 | 22.4 | 18. | −4.3 | 23.9 | −1.0 | 17.5 | 21.8 | −9.0 | 17.5 | −2.6 | | |
| | Median | 35.2 | 2 | 0.5 | 32.0 | 0.0 | 32.5 | 30.3 | 0.0 | 27.7 | 0.0 | | |
| | Q3 | 48.4 | 40.4 | 13.4 | 45.2 | 6.3 | 46.3 | 41.5 | 10.6 | 40.9 | 7.5 | | |
| | Min/Max | 0/92 | 0/74 | −31/54 | 0/75 | −41/41 | 0/82 | 0/79 | −56/63 | 0/78 | −43/54 | | |
| Y + 10 | n | 173 | 173 | 173 | 173 | 173 | 170 | 170 | 170 | 170 | l70 | 0.0415 | 0.6693 |
| | Mean | 36.0 | 30.4 | 5.5 | 33.4 | 2.5 | 32.9 | 31.2 | 1.7 | 30.4 | 2.5 | | |
| | Std Dev | 18.31 | 15.41 | 16.70 | 15.72 | 12.74 | 20.03 | 15.26 | 17.50 | 18.27 | 14.12 | | |
| | Q1 | 25.0 | 18.5 | −4.3 | 23.9 | −0.5 | 17.1 | 21.8 | −6.9 | 17.5 | −2.6 | | |
| | Median | 37.2 | 28.7 | 0.0 | 32.0 | 0.0 | 32.2 | 30.3 | 0.0 | 27.7 | 0.0 | | |
| | Q3 | 49.4 | 40.4 | 17.0 | 45.2 | 7.9 | 46.6 | 41.5 | 9.6 | 40.9 | 7.9 | | |
| | Min/Max | 0/92 | 0/74 | −31/54 | 0/75 | −50/40 | 0/81 | 0/79 | −53/63 | 0/78 | −59/54 | | |

[a] Visits Y and Y + 2 vales for only those subjects present at Visit Y + x are shown in this column.
[b] between treatments in Change from Visit Y assessed using ANCOVA with treatment as a categorical Factor and Visit Y or Y + 2 value as covariate.

TABLE 11.4.16
WOMAC Osteoarthritis Index—Maintenance Phase Imputed Values
ITT Population
WOMAC Composite Score

| | | Placebo | | | | | Kadian NT | | | | | | |
|---|---|---|---|---|---|---|---|---|---|---|---|---|---|
| | | N = 173 | | | | | N = 170 | | | | | P-value [c] | |
| | | | | Change | | Change | | | Change | | Change | Change | Change |
| | | Actual | Visit | From | Visit | From | Actual | Visit | From | Visit | From | From | From |
| | | Value | Y | Visit | Y + 2 | Visit | Value | Y | Visit | Y + 2 | Visit | Visit | Visit |
| Visit | Statistic | [a] | [b] | Y | [b] | Y + 2 | [a] | [b] | Y | [b] | Y + 2 | Y | Y + 2 |
| Y + 12 | n | 173 | 173 | 173 | 173 | 173 | 170 | 170 | 170 | 170 | 170 | 0.0312 | 0.5309 |
| | Mean | 36.2 | 30.4 | 5.8 | 33.4 | 2.8 | 32.8 | 31.2 | 1.6 | 30.4 | 2.4 | | |
| | Std Dev | 18.30 | 15.41 | 16.83 | 15.72 | 12.56 | 19.98 | 15.26 | 18.04 | 18.27 | 14.24 | | |
| | Q1 | 25.0 | 18.5 | −4.2 | 23.9 | −0.1 | 16.0 | 21.8 | −6.8 | 17.5 | −3.7 | | |
| | Median | 36.2 | 28.7 | 0.3 | 32.0 | 0.0 | 32.7 | 30.3 | 0.0 | 27.7 | 0.0 | | |
| | Q3 | 49.5 | 40.4 | 17.0 | 45.2 | 6.9 | 45.3 | 41.5 | 12.2 | 40.9 | 9.0 | | |
| | Min/Max | 0/92 | 0/74 | −29/55 | 0/75 | −39/40 | 0/81 | 0/79 | −59/63 | 0/78 | −42/54 | | |

[a] Visits Y and Y + 2 values for only those subjects present at Visit Y + x are shown in this column.
[b] Differences between treatments in Change from Visit Y assessed using ANCOVA with treatment as a categorical Factor and Visit Y or Y + 2 value as covariate.

TABLE 11.4.16
WOMAC Osteoarthritis Index—Maintenance Phase Imputed Values
ITT Population
WOMAC Composite Score

| | | Placebo | | | | | Kadian NT | | | | | | |
|---|---|---|---|---|---|---|---|---|---|---|---|---|---|
| | | N = 173 | | | | | N = 170 | | | | | P-value [c] | |
| | | | | Change | | Change | | | Change | | Change | Change | Change |
| | | Actual | Visit | From | Visit | From | Actual | Visit | From | Visit | From | From | From |
| | | Value | Y | Visit | Y + 2 | Visit | Value | Y | Visit | Y + 2 | Visit | Visit | Visit |
| Visit | Statistic | [a] | [b] | Y | [b] | Y + 2 | [a] | [b] | Y | [b] | Y + 2 | Y | Y + 2 |
| Y | n | 173 | | | | | 170 | | | | | | |
| | Mean | 29.4 | | | | | 29.7 | | | | | | |
| | Std Dev | 15.62 | | | | | 15.47 | | | | | | |
| | Q1 | 20.0 | | | | | 20.0 | | | | | | |
| | Median | 25.0 | | | | | 30.0 | | | | | | |
| | Q3 | 40.0 | | | | | 40.0 | | | | | | |
| | Min/Max | 0/75 | | | | | 0/80 | | | | | | |
| Y + 2 | n | 173 | 173 | 173 | | | 170 | 170 | 170 | | | 0.0026 | |
| | Mean | 33.0 | 29.4 | 3.6 | | | 28.8 | 29.7 | −1.0 | | | | |
| | Std Dev | 16.05 | 15.62 | 13.62 | | | 17.46 | 15.47 | 15.69 | | | | |
| | Q1 | 25.0 | 20.0 | −5.0 | | | 15.0 | 20.0 | −10.0 | | | | |
| | Median | 30.0 | 25.0 | 0.0 | | | 25.0 | 30.0 | 0.0 | | | | |
| | Q3 | 45.0 | 40.0 | 10.0 | | | 40.0 | 40.0 | 5.0 | | | | |
| | Min/Max | 0/75 | 0/75 | −25/40 | | | 0/80 | 0/80 | −65/65 | | | | |
| Y + 4 | n | 173 | 173 | 173 | 173 | 173 | 170 | 170 | 170 | 170 | 170 | 0.0222 | 0.9964 |
| | Mean | 33.7 | 29.4 | 4.2 | 33.0 | 0.7 | 30.1 | 29.7 | 0.4 | 28.8 | 1.4 | | |
| | Std Dev | 17.65 | 15.62 | 15.86 | 16.05 | 10.41 | 17.73 | 15.47 | 16.74 | 17.46 | 11.83 | | |
| | Q1 | 20.0 | 20.0 | −5.0 | 25.0 | −5.0 | 20.0 | 20.0 | −10.0 | 15.0 | 0.0 | | |
| | Median | 35.0 | 25.0 | 0.0 | 30.0 | 0.0 | 30.0 | 30.0 | 0.0 | 25.0 | 0.0 | | |
| | Q3 | 45.0 | 40.0 | 15.0 | 45.0 | 5.0 | 40.0 | 40.0 | 10.0 | 40.0 | 5.0 | | |
| | Min/Max | 0/85 | 0/75 | −35/45 | 0/75 | −35/30 | 0/80 | 0/80 | −65/65 | 0/80 | −40/55 | | |

[a] Visits Y and Y + 2 vales for only those subjects present at Visit Y + x are shown in this column.
[b] Differences between treatments in Change from Visit Y assessed using ANCOVA with treatment as a categorical Factor and Visit Y or Y + 2 value as covariate.

TABLE 11.4.16
WOMAC Osteoarthritis Index—Maintenance Phase Imputed Values
ITT Population
WOMAC Composite Score

| | | Placebo | | | | | Kadian NT | | | | | | |
|---|---|---|---|---|---|---|---|---|---|---|---|---|---|
| | | N = 173 | | | | | N = 170 | | | | | P-value [c] | |
| | | | | Change | | Change | | | Change | | Change | Change | Change |
| | | Actual | Visit | From | Visit | From | Actual | Visit | From | Visit | From | From | From |
| | | Value | Y | Visit | Y + 2 | Visit | Value | Y | Visit | Y + 2 | Visit | Visit | Visit |
| Visit | Statistic | [a] | [b] | Y | [b] | Y + 2 | [a] | [b] | Y | [b] | Y + 2 | Y | Y + 2 |
| Y + 6 | n | 173 | 173 | 173 | 173 | 173 | 170 | 170 | 170 | 170 | 170 | 0.0781 | 0.5386 |
| | Mean | 33.9 | 29.4 | 4.5 | 33.0 | 0.9 | 31.1 | 29.7 | 1.4 | 28.8 | 2.4 | | |
| | Std Dev | 17.13 | 15.62 | 15.24 | 16.05 | 10.52 | 18.82 | 15.47 | 17.91 | 17.46 | 12.89 | | |
| | Q1 | 25.0 | 20.0 | −5.0 | 25.0 | −5.0 | 20.0 | 20.0 | −10.0 | 15.0 | −5.0 | | |
| | Median | 35.0 | 25.0 | 0.0 | 30.0 | 0.0 | 30.0 | 30.0 | 0.0 | 25.0 | 0.0 | | |
| | Q3 | 45.0 | 40.0 | 15.0 | 45.0 | 5.0 | 40.0 | 40.0 | 10.0 | 40.0 | 10.0 | | |
| | Min/Max | 0/90 | 0/75 | −35/50 | 0/75 | −35/30 | 0/85 | 0/80 | −60/65 | 0/80 | −35/55 | | |
| Y + 8 | n | 173 | 173 | 173 | 173 | 173 | 170 | 170 | 170 | 170 | 170 | 0.1163 | 0.6166 |
| | Mean | 34.0 | 29.4 | 4.8 | 33.0 | 1.0 | 31.3 | 29.7 | 1.6 | 28.8 | 2.6 | | |
| | Std Dev | 18.13 | 15.62 | 16.19 | 16.05 | 12.88 | 19.84 | 15.47 | 19.16 | 17.46 | 14.97 | | |
| | Q1 | 20.0 | 20.0 | −5.0 | 25.0 | 0.0 | 15.0 | 20.0 | −10.0 | 15.0 | −5.0 | | |
| | Median | 35.0 | 25.0 | 0.0 | 30.0 | 0.0 | 30.0 | 30.0 | 0.0 | 25.0 | 0.0 | | |
| | Q3 | 45.0 | 40.0 | 15.0 | 45.0 | 5.0 | 45.0 | 40.0 | 10.0 | 40.0 | 10.0 | | |
| | Min/Max | 0/90 | 0/75 | −35/50 | 0/75 | −45/45 | 0/85 | 0/80 | −70/65 | 0/80 | −40/60 | | |
| Y + 10 | n | 173 | 173 | 173 | 173 | 173 | 170 | 170 | 170 | 170 | 170 | 0.0597 | 0.8759 |
| | Mean | 34.8 | 29.4 | 5.3 | 33.0 | 1.8 | 31.6 | 29.7 | 1.8 | 28.8 | 2.8 | | |
| | Std Dev | 18.17 | 15.62 | 16.74 | 16.05 | 12.69 | 20.15 | 15.47 | 18.63 | 17.46 | 15.01 | | |
| | Q1 | 25.0 | 20.0 | −5.0 | 25.0 | −5.0 | 15.0 | 20.0 | −10.0 | 15.0 | −5.0 | | |
| | Median | 35.0 | 25.0 | 0.0 | 30.0 | 0.0 | 30.0 | 30.0 | 0.0 | 25.0 | 0.0 | | |
| | Q3 | 45.0 | 40.0 | 15.0 | 45.0 | 10.0 | 45.0 | 40.0 | 10.0 | 40.0 | 10.0 | | |
| | Min/Max | 0/90 | 0/75 | −35/50 | 0/75 | −50/40 | 0/85 | 0/80 | −60/80 | 0/80 | −55/60 | | |

[a] Visits Y and Y + 2 values for only those sujects present at Visit Y + x are shown in this column.
[b] Differences between treatments in Change from Visit Y assessed using ANCOVA with treatment as a categorical Factor and Visit Y or Y + 2 value as covariate.

TABLE 11.4.16
WOMAC Osteoarthritis Index—Maintenance Phase Imputed Values
ITT Population
WOMAC Composite Score

| | | Placebo | | | | | Kadian NT | | | | | | |
|---|---|---|---|---|---|---|---|---|---|---|---|---|---|
| | | N = 173 | | | | | N = 170 | | | | | P-value [c] | |
| | | | | Change | | Change | | | Change | | Change | Change | Change |
| | | Actual | Visit | From | Visit | From | Actual | Visit | From | Visit | From | From | From |
| | | Value | Y | Visit | Y + 2 | Visit | Value | Y | Visit | Y + 2 | Visit | Visit | Visit |
| Visit | Statistic | [a] | [b] | Y | [b] | Y + 2 | [a] | [b] | Y | [b] | Y + 2 | Y | Y + 2 |
| Y + 12 | n | 173 | 173 | 173 | 173 | 173 | 170 | 170 | 170 | 170 | 170 | 0.0229 | 0.7094 |
| | Mean | 35.1 | 29.4 | 5.7 | 33.0 | 2.1 | 31.1 | 29.7 | 1.4 | 28.8 | 2.4 | | |
| | Std Dev | 18.28 | 15.62 | 17.07 | 16.05 | 12.70 | 19.87 | 15.47 | 18.91 | 17.46 | 14.94 | | |
| | Q1 | 25.0 | 20.0 | −6.0 | 25.0 | 0.0 | 15.0 | 20.0 | −10.0 | 16.0 | −5.0 | | |
| | Median | 35.0 | 25.0 | 0.0 | 30.0 | 0.0 | 30.0 | 30.0 | 0.0 | 25.0 | 8.0 | | |
| | Q3 | 50.8 | 40.0 | 15.0 | 45.0 | 5.0 | 45.0 | 40.0 | 10.0 | 40.0 | 10.0 | | |
| | Min/Max | 0/90 | 0/75 | −35/50 | 0/75 | −30/40 | 0/85 | 0/80 | −70/65 | 0/80 | −35/60 | | |

[a] Visits Y and Y + 2 values for only those subjects present at Visit Y + x are shown in this column.
[b] Differences between treatments in Change from Visit Y assessed using ANCOVA with treatment as a categorical Factor and Visit Y or Y + 2 value as covariate.

TABLE 11.4.16
WOMAC Osteoarthritis Index Maintenance Phase Imputed Values
ITT Population
WOMAC Composite Score

| | | Placebo | | | | | Kadian NT | | | | | | |
|---|---|---|---|---|---|---|---|---|---|---|---|---|---|
| | | N = 173 | | | | | N = 170 | | | | | P-value [c] | |
| | | | | Change | | Change | | | Change | | Change | Change | Change |
| | | Actual | Visit | From | Visit | From | Actual | Visit | From | Visit | From | From | From |
| | | Value | Y | Visit | Y + 2 | Visit | Value | Y | Visit | Y + 2 | Visit | Visit | Visit |
| Visit | Statistic | [a] | [b] | Y | [b] | Y + 2 | [a] | [b] | Y | [b] | Y + 2 | Y | Y + 2 |
| Y | n | 173 | | | | | 170 | | | | | | |
| | Mean | 34.5 | | | | | 35.1 | | | | | | |
| | Std Dev | 18.87 | | | | | 18.41 | | | | | | |
| | Q1 | 25.0 | | | | | 25.0 | | | | | | |
| | Median | 25.0 | | | | | 37.5 | | | | | | |
| | Q3 | 50.0 | | | | | 50.0 | | | | | | |
| | Min/Max | 0/88 | | | | | 0/75 | | | | | | |
| Y + 2 | n | 173 | 173 | 173 | | | 170 | 170 | 170 | | | 0.1652 | |
| | Mean | 36.2 | 34.5 | 1.7 | | | 34.2 | 35.1 | −0.9 | | | | |
| | Std Dev | 17.42 | 18.87 | 16.44 | | | 20.15 | 18.41 | 18.57 | | | | |
| | Q1 | 25.0 | 25.0 | −12.5 | | | 25.0 | 25.0 | −12.5 | | | | |
| | Median | 37.5 | 25.0 | 0.0 | | | 25.0 | 37.5 | 0.0 | | | | |
| | Q3 | 50.0 | 50.0 | 12.5 | | | 50.0 | 50.0 | 12.5 | | | | |
| | Min/Max | 0/75 | 0/88 | −25/50 | | | 0/88 | 0/75 | −75/63 | | | | |
| Y + 4 | n | 173 | 173 | 173 | 173 | 173 | 170 | 170 | 170 | 170 | 170 | 0.0908 | 0.3686 |
| | Mean | 37.9 | 34.5 | 3.5 | 36.2 | 1.7 | 35.0 | 35.1 | −0.1 | 34.2 | 0.8 | | |
| | Std Dev | 19.90 | 18.87 | 19.46 | 17.42 | 14.22 | 20.69 | 18.41 | 20.15 | 20.15 | 14.01 | | |
| | Q1 | 25.0 | 25.0 | 0.0 | 25.0 | 0.0 | 25.0 | 25.0 | −12.5 | 25.0 | 0.0 | | |
| | Median | 37.5 | 25.0 | 0.0 | 37.5 | 0.0 | 37.5 | 37.5 | 0.0 | 25.0 | 0.0 | | |
| | Q3 | 50.0 | 50.0 | 12.5 | 50.0 | 12.5 | 50.0 | 50.0 | 12.5 | 50.0 | 12.5 | | |
| | Min/Max | 0/75 | 0/88 | −50/75 | 0/75 | −38/75 | 0/88 | 0/75 | −75/63 | 0/88 | −38/63 | | |

[a] Visits Y and Y + 2 values for only those subjects present at Visit Y + x are shown on in this column.
[b] Differences between treatments in Change from Visit Y assessed using ANCOVA with treatment as a categorical Factor and Visit Y or Y + 2 value as covariate.

TABLE 11.4.16
WOMAC Osteoarthritis Index—Maintenance Phase Imputed Values
ITT Population
WOMAC Composite Score

| | | Placebo | | | | | Kadian NT | | | | | | |
|---|---|---|---|---|---|---|---|---|---|---|---|---|---|
| | | N = 173 | | | | | N = 170 | | | | | P-va1ue [c] | |
| | | | | Change | | Change | Visit | | Change | | Change | Change | Change |
| | | Actual | Visit | From | Visit | From | Actual | Visit | From | Visit | From | From | From |
| | | Value | Y | Visit | Y + 2 | Visit | Value | Y | Visit | Y + 2 | Visit | Visit | Visit |
| Visit | Statistic | [a] | [b] | Y | [b] | Y + 2 | [a] | [b] | Y | [b] | Y + 2 | Y | Y + 2 |
| Y + 6 | n | 173 | 173 | 173 | 173 | 173 | 170 | 170 | 170 | 170 | 170 | 0.2434 | 0.7695 |
| | Mean | 38.2 | 34.5 | 3.8 | 36.2 | 2.0 | 36.2 | 35.1 | 1.1 | 34.2 | 2.0 | | |
| | Std Dev | 20.78 | 18.87 | 21.19 | 17.42 | 15.23 | 21.24 | 18.41 | 20.25 | 20.15 | 15.88 | | |
| | Q1 | 25.0 | 25.0 | 12.5 | 25.0 | 0.0 | 25.0 | 25.0 | −12.5 | 25.0 | 0.0 | | |
| | Median | 37.5 | 25.0 | 0.0 | 37.5 | 0.0 | 37.5 | 37.5 | 0.0 | 25.0 | 0.0 | | |
| | Q3 | 50.0 | 50.0 | 12.5 | 50.0 | 12.5 | 50.0 | 50.0 | 12.5 | 50.0 | 12.5 | | |
| | Min/Max | 0/10 | 0/88 | −38/88 | 0/75 | −38/75 | 0/88 | 0/75 | −75/63 | 0/88 | −75/63 | | |
| Y + 8 | n | 173 | 173 | 173 | 173 | 173 | 170 | 170 | 170 | 170 | 170 | 0.1502 | 0.5375 |
| | Mean | 38.6 | 34.5 | 4.1 | 36.2 | 2.4 | 36.0 | 35.1 | 0.9 | 34.2 | 1.8 | | |
| | Std Dev | 20.44 | 18.87 | 20.54 | 17.42 | 15.74 | 22.64 | 18.41 | 21.00 | 20.15 | 16.56 | | |
| | Q1 | 25.0 | 25.0 | −12.5 | 25.0 | 0.0 | 25.0 | 25.0 | −12.5 | 25.0 | 0.0 | | |
| | Median | 37.5 | 25.0 | 0.0 | 37.5 | 0.0 | 37.5 | 37.5 | 0.0 | 25.0 | 0.0 | | |
| | Q3 | 50.0 | 50.0 | 12.5 | 50.0 | 12.5 | 50.0 | 50.0 | 12.5 | 50.0 | 12.5 | | |
| | Min/Max | 0/88 | 0.88 | −38/75 | 0/75 | −38/75 | 0/88 | 0/75 | −75/63 | 0/88 | −75/63 | | |
| Y + 10 | n | 173 | 173 | 173 | 173 | 173 | 170 | 170 | 170 | 170 | 170 | 0.1261 | 0.4329 |
| | Mean | 38.9 | 34.5 | 4.4 | 36.2 | 2.7 | 36.0 | 35.1 | 0.9 | 34.2 | 1.8 | | |
| | Std Dev | 20.99 | 18.87 | 21.72 | 17.42 | 17.40 | 22.72 | 18.41 | 21.09 | 20.15 | 17.85 | | |
| | Q1 | 25.0 | 25.0 | −12.5 | 25.0 | 0.0 | 25.0 | 25.0 | −12.5 | 25.0 | 0.0 | | |
| | Median | 37.5 | 25.0 | 0.0 | 37.5 | 0.0 | 37.5 | 37.5 | 0.0 | 25.0 | 0.0 | | |
| | Q3 | 50.0 | 50.0 | 12.5 | 50.0 | 12.5 | 50.0 | 50.0 | 12.5 | 50.0 | 12.5 | | |
| | Min/Max | 0/88 | 0/88 | −50/75 | 0/75 | −38/75 | 0/88 | 0/75 | −63/63 | 0/88 | −75/63 | | |

[a] Visits Y and Y + 2 values for only those subjects present at Visit Y + x are shown in this column.
[b] Differences between treatments in Change from Visit Y assessed using ANCOVA with treatment as a categorical Factor and Visit Y or Y + 2 value as covariate.

TABLE 11.4.16
WOMAC Osteoarthritis Index—Maintenance Phase Imputed Values
ITT Population
WOMAC Composite Score

| | | Placebo | | | | | Kadian NT | | | | | | |
|---|---|---|---|---|---|---|---|---|---|---|---|---|---|
| | | N = 173 | | | | | N = 170 | | | | | P-value [c] | |
| | | | | Change | | Change | | | Change | | Change | Change | Change |
| | | Actual | Visit | From | Visit | From | Actual | Visit | From | Visit | From | From | From |
| | | Value | Y | Visit | Y + 2 | Visit | Value | Y | Visit | Y + 2 | Visit | Visit | Visit |
| Visit | Statistic | [a] | [b] | Y | [b] | Y + 2 | [a] | [b] | Y | [b] | Y + 2 | Y | Y + 2 |
| Y + 12 | n | 173 | 173 | 173 | 173 | 173 | 170 | 170 | 170 | 170 | 170 | 0.0625 | 0.2355 |
| | Mean | 39.8 | 34.5 | 5.3 | 36.2 | 3.6 | 36.2 | 35.1 | 1.1 | 34.2 | 2.0 | | |
| | Std Dev | 21.01 | 18.87 | 21.99 | 17.42 | 16.66 | 22.47 | 18.41 | 21.06 | 20.15 | 17.85 | | |
| | Q1 | 25.0 | 25.0 | −12.5 | 25.0 | 0.0 | 25.0 | 25.0 | −12.5 | 25.0 | 0.0 | | |
| | Median | 37.5 | 25.0 | 0.0 | 37.5 | 0.0 | 37.5 | 37.5 | 0.0 | 25.0 | 0.0 | | |
| | Q3 | 50.0 | 50.0 | 12.5 | 50.0 | 12.5 | 50.0 | 50.0 | 12.5 | 50.0 | 12.5 | | |
| | Min/Max | 0/88 | 0/88 | −38/75 | 0/75 | −38/75 | 0/88 | 0/75 | −63/63 | 0/88 | −63/63 | | |

[a] Visits Y and Y + 2 values for only those subjects present at Visit Y + x are shown in this column.
[b] Differences between treatments in Change from Visit Y assessed using ANCOVA with treatment as a categorical Factor and Visit Y or Y + 2 value as covariate.

TABLE 11.4.16
WOMAC Osteoarthritis Index—Maintenance Phase ImputedValues
ITT Population
WOMAC Composite Score

| | | Placebo | | | | | Kadian NT | | | | | | |
|---|---|---|---|---|---|---|---|---|---|---|---|---|---|
| | | N = 173 | | | | | N = 170 | | | | | P-value [c] | |
| | | | | Change | | Change | | | Change | | Change | Change | Change |
| | | Actual | Visit | From | Visit | From | Actual | Visit | From | Visit | From | From | From |
| | | Value | Y | Visit | Y + 2 | Visit | Value | Y | Visit | Y + 2 | Visit | Visit | Visit |
| Visit | Statistic | [a] | [b] | Y | [b] | Y + 2 | [a] | [b] | Y | [b] | Y + 2 | Y | Y + 2 |
| Y | n | 173 | | | | | 170 | | | | | | |
| | Mean | 29.3 | | | | | 30.7 | | | | | | |
| | Std Dev | 16.40 | | | | | 16.31 | | | | | | |
| | Q1 | 17.6 | | | | | 22.1 | | | | | | |
| | Median | 26.5 | | | | | 30.9 | | | | | | |
| | Q3 | 39.7 | | | | | 42.6 | | | | | | |
| | Min/Max | 0/75 | | | | | 0/82 | | | | | | |
| Y + 2 | n | 173 | 173 | 173 | | | 170 | 170 | 170 | | | 0.0490 | |
| | Mean | 32.3 | 29.3 | 3.0 | | | 30.1 | 30.7 | −0.6 | | | | |
| | Std Dev | 17.52 | 16.40 | 15.07 | | | 20.55 | 16.31 | 16.34 | | | | |
| | Q1 | 22.1 | 17.6 | −5.9 | | | 13.2 | 22.1 | −8.8 | | | | |
| | Median | 29.4 | 26.5 | 0.0 | | | 25.7 | 30.9 | 0.0 | | | | |
| | Q3 | 45.6 | 39.7 | 10.3 | | | 44.1 | 42.6 | 4.4 | | | | |
| | Min/Max | 0/81 | 0/75 | −41/51 | | | 0/85 | 0/82 | −37/69 | | | | |
| Y + 4 | n | 173 | 173 | 173 | 173 | 173 | 170 | 170 | 170 | 170 | 170 | 0.0470 | 0.6363 |
| | Mean | 33.7 | 29.3 | 4.4 | 32.3 | 1.4 | 31.3 | 30.7 | 0.7 | 30.1 | 1.2 | | |
| | Std Dev | 19.07 | 16.40 | 16.55 | 17.52 | 11.84 | 19.38 | 16.31 | 16.08 | 20.55 | 11.29 | | |
| | Q1 | 20.6 | 17.6 | −5.9 | 22.1 | 0.0 | 14.7 | 22.1 | −7.4 | 13.2 | −1.5 | | |
| | Median | 30.9 | 26.5 | 0.0 | 29.4 | 0.0 | 28.7 | 30.9 | 0.0 | 25.7 | 0.0 | | |
| | Q3 | 47.1 | 39.7 | 13.2 | 45.6 | 4.4 | 44.1 | 42.6 | 7.4 | 44.1 | 4.4 | | |
| | Min/Max | 0/87 | 0/75 | −26/63 | 0/81 | −50/43 | 0/85 | 0/82 | −41/69 | 0/85 | −57/56 | | |

[a] Visits Y and Y + 2 values for only those subjects present at Visit Y + x are shown in this column.
[b] Differences between treatments in Change from Visit Y assessed using ANCOVA with treatment as a categorical Factor and Visit Y or Y + 2 value as covariate.

TABLE 11.4.16
WOMAC Osteoarthritis Index—Maintenance Phase Imputed Values
ITT Population
WOMAC Composite Score

| | | Placebo | | | | | Kadian NT | | | | | | |
|---|---|---|---|---|---|---|---|---|---|---|---|---|---|
| | | N = 173 | | | | | N = 170 | | | | | P-value [c] | |
| | | | | Change | | Change | | | Change | | Change | Change | Change |
| | | Actual | Visit | From | Visit | From | Actual | Visit | From | Visit | From | From | From |
| | | Value | Y | Visit | Y + 2 | Visit | Value | Y | Visit | Y + 2 | Visit | Visit | Visit |
| Visit | Statistic | [a] | [b] | Y | [b] | Y + 2 | [a] | [b] | Y | [b] | Y + 2 | Y | Y + 2 |
| Y + 6 | n | 173 | 173 | 173 | 173 | 173 | 170 | 170 | 170 | 170 | 170 | 0.0103 | 0.2288 |
| | Mean | 34.8 | 29.3 | 5.5 | 32.3 | 2.5 | 31.4 | 30.7 | 0.8 | 30.1 | 1.3 | | |
| | Std Dev | 19.35 | 16.40 | 16.28 | 17.52 | 12.08 | 19.90 | 16.31 | 16.23 | 20.55 | 12.37 | | |
| | Q1 | 22.1 | 17.6 | −2.9 | 22.1 | 0.0 | 17.6 | 22.1 | −7.4 | 13.2 | −1.5 | | |
| | Median | 32.4 | 26.5 | 1.5 | 29.4 | 0.0 | 28.7 | 30.9 | 0.0 | 25.7 | 0.0 | | |
| | Q3 | 48.5 | 39.7 | 16.2 | 45.6 | 7.4 | 44.1 | 42.6 | 8.8 | 44.1 | 5.9 | | |
| | Min/Max | 0/96 | 0/75 | −26/63 | 0/81 | −51/43 | 0/88 | 0/82 | −51/69 | 0/85 | −60/56 | | |
| Y + 8 | n | 173 | 173 | 173 | 173 | 173 | 170 | 170 | 170 | 170 | 170 | 0.0525 | 0.6168 |
| | Mean | 34.9 | 29.3 | 5.6 | 32.3 | 2.6 | 32.4 | 30.7 | 1.7 | 30.1 | 2.2 | | |
| | Std Dev | 20.45 | 16.40 | 17.24 | 17.52 | 13.36 | 20.81 | 16.31 | 17.64 | 20.55 | 14.46 | | |
| | Q1 | 19.1 | 17.6 | −2.9 | 22.1 | 0.0 | 16.2 | 22.1 | −7.4 | 13.2 | −1.5 | | |
| | Median | 33.8 | 26.5 | 0.0 | 29.4 | 0.0 | 31.6 | 30.9 | 0.0 | 25.7 | 0.0 | | |
| | Q3 | 50.0 | 39.7 | 16.2 | 45.6 | 7.4 | 45.6 | 42.6 | 10.3 | 44.1 | 7.4 | | |
| | Min/Max | 0/96 | 0/75 | −28/65 | 0/81 | −51/46 | 0/88 | 0/82 | −53/69 | 0/85 | −62/56 | | |
| Y + 10 | n | 173 | 173 | 173 | 173 | 173 | 170 | 170 | 170 | 170 | 170 | 0.0262 | 0.3699 |
| | Mean | 35.7 | 29.3 | 6.4 | 32.3 | 3.3 | 32.6 | 30.7 | 1.9 | 30.1 | 2.5 | | |
| | Std Dev | 19.83 | 16.40 | 17.69 | 17.52 | 13.52 | 20.69 | 16.31 | 17.11 | 20.55 | 14.23 | | |
| | Q1 | 23.5 | 17.6 | −2.9 | 22.1 | 0.0 | 16.2 | 22.1 | −5.9 | 13.2 | −1.5 | | |
| | Median | 35.3 | 26.5 | 0.0 | 29.4 | 0.0 | 30.9 | 30.9 | 0.0 | 25.7 | 0.0 | | |
| | Q3 | 48.5 | 39.7 | 17.6 | 45.6 | 7.4 | 45.6 | 42.6 | 8.8 | 44.1 | 7.4 | | |
| | Min/Max | 0/96 | 0/75 | −28/65 | 0/81 | −57/50 | 0/87 | 0/82 | −43/69 | 0/85 | −62/56 | | |

[a] Visits Y and Y + 2 values for only those subjects present at Visit Y + x are shown in this column.
[b] Differences between treatments in Change from Visit Y assessed using ANCOVA with treatment as a categorical Factor and Visit Y or Y + 2 a as covariate.

TABLE 11.4.16
WOMAC Osteoarthritis Index—Maintenance Phase Imputed Values
ITT Population
WOMAC Composite Score

| | | Placebo | | | | | Kadian NT | | | | | | |
|---|---|---|---|---|---|---|---|---|---|---|---|---|---|
| | | N = 173 | | | | | N = 170 | | | | | P-value [c] | |
| | | | | Change | | Change | | | Change | | Change | Change | Change |
| | | Actual | Visit | From | Visit | From | Actual | Visit | From | Visit | From | From | From |
| | | Value | Y | Visit | Y + 2 | Visit | Value | Y | Visit | Y + 2 | Visit | Visit | Visit |
| Visit | Statistic | [a] | [b] | Y | [b] | Y + 2 | [a] | [b] | Y | [b] | Y + 2 | Y | Y + 2 |
| Y + 12 | n | 173 | 173 | 173 | 173 | 173 | 170 | 170 | 170 | 170 | 170 | 0.0641 | 0.5955 |
| | Mean | 35.5 | 29.3 | 6.2 | 32.3 | 3.1 | 32.9 | 30.7 | 2.3 | 30.1 | 2.8 | | |
| | Std Dev | 19.81 | 16.40 | 17.82 | 17.52 | 13.43 | 21.06 | 16.31 | 18.43 | 20.55 | 14.93 | | |
| | Q1 | 23.5 | 17.6 | −2.9 | 22.1 | 0.0 | 16.2 | 22.1 | −7.4 | 13.2 | −2.9 | | |
| | Median | 35.3 | 26.5 | 1.5 | 29.4 | 0.0 | 32.4 | 30.9 | 0.0 | 25.7 | 0.0 | | |
| | Q3 | 50.0 | 39.7 | 16.2 | 45.6 | 7.4 | 45.6 | 42.6 | 10.3 | 44.1 | 7.4 | | |
| | Min/Max | 0/96 | 0/75 | −25/65 | 0/81 | −50/50 | 0/87 | 0/82 | −53/69 | 0/85 | −44/57 | | |

[a] Visits Y and Y + 2 values for only those subjects present at Visit Y + x are shown in this column.
[b] Differences between treatments in Change from Visit Y assessed using ANCOVA with treatment as a categorical Factor and Visit Y or Y + 2 value as covariate.

While the present invention has been described in terms of the preferred embodiments, it is understood that variations and modifications will occur to those skilled in the art. Therefore, it is intended that the appended claims cover all such equivalent variations that come within the scope of the invention as claimed.

What is claimed is:

1. A method of treating a condition in a host that is responsive to an agonist, the method comprising administering a multi-layer pharmaceutical composition comprising an agonist and an antagonist thereof that are not in direct contact with one another in the intact form of the composition, wherein administration of the intact form of the composition to the host effectively treats the condition in a manner more efficacious than placebo when measure using the Brief Pain Inventory.

2. The method of claim 1 wherein the host is treated for up to twelve weeks.

3. A method of treating a condition in a host that is responsive to an agonist, the method comprising administering a multi-layer pharmaceutical composition comprising an agonist and an antagonist thereof that are not in direct contact with one another in the intact form of the composition, wherein administration of the intact form of the composition to the host effectively treats the condition in a manner more efficacious than placebo when measured using the WOMAC Osteoarthitis Index.

4. The method of claim 4 wherein the host is treated for up to twelve weeks.
